(19) United States
(12) Patent Application Publication
(10) Pub. No.: US 2023/0212583 A1
(43) Pub. Date: Jul. 6, 2023

(54) ANTISENSE THERAPEUTICS FOR THE TREATMENT OF CORONAVIRUS

(71) Applicants: Oregon State University, Corvallis, OR (US); Philipps-Universität Marburg, Marburg (DE)

(72) Inventors: Eva Friebertshäuser, Marburg (DE); Hong M. Moulton, Corvallis, OR (US); David A. Stein, Corvallis, OR (US)

(73) Assignees: Oregon State University, Corvallis, OR (US); Philipps-Universität Marburg, Marburg, OR (DE)

(21) Appl. No.: 18/046,235

(22) Filed: Oct. 13, 2022

Related U.S. Application Data
(63) Continuation of application No. PCT/US21/27066, filed on Apr. 13, 2021.
(60) Provisional application No. 63/009,574, filed on Apr. 14, 2020.

(51) Int. Cl. C12N 15/113; A61P 31/14

(52) U.S. Cl. CPC C12N 15/1137; A61P 31/14; C12N 2310/31; C12N 2310/11

(57) ABSTRACT

Disclosed herein are embodiments of a compound useful for treating or preventing SARS-Cov-2 infections. Also disclosed is a method for administering the compound to a subject, particularly a human subject, to treat or prevent a SARS-CoV-2 infection in the subject. The compound may comprise an oligomer comprising a nucleic acid base sequence that is antisense to a gene, pre-mRNA or mRNA in the subject, such as a gene, pre-mRNA or mRNA of transmembrane serine protease 2 (TMPRSS2). The compound also may comprise a peptide sequence. In some embodiments, the compound is a peptide-conjugated phosphorodiamidate morpholino oligomer (PPMO).

### CROSS REFERENCE TO RELATED APPLICATION

This application is a continuation of International Application No. PCT/US2021/027066, filed on Apr. 13, 2021, which claims the benefit of the earlier filing date of U.S. provisional patent application No. 63/009,574, filed Apr. 14, 2020, both of which are incorporated herein by reference in its entirety.

### FIELD

This disclosure concerns embodiments of a compound useful for treating or preventing SARS-CoV-2 infections and a method of using the same.

### BACKGROUND

In December 2019, cases of an acute respiratory disease were reported from Wuhan, the capitol of Hubei province in China. The number of infections increased rapidly and spread to other areas of China and on Jan. 13, 2020, the first case was reported outside of China. The causative agent was identified as a novel coronavirus (CoV) of the lineage b of the genus Betacoronavirus that also includes the 2002 SARS-CoV that caused a global outbreak of severe acute respiratory syndrome (SARS) in 2002 and 2003. The newly-emerged CoV was named SARS-CoV-2 by the World Health Organization (WHO) in February 2020, and the outbreak was declared as pandemic on Mar. 11, 2020. The respiratory disease caused by SARS-CoV-2 was named coronavirus 2019 disease (COVID-19). As of Apr. 8, 2021, 221 countries have reported 133 million cases and 2.9 million deaths.

### SUMMARY

Disclosed herein are embodiments of a method for treating or preventing a SARS-CoV-2 infection is a subject, such as a human or non-human subject, comprising administering a compound comprising a steric blocking antisense oligomer. The antisense oligomer may comprise a nucleic acid base sequence that is antisense to at least a portion of the transmembrane serine protease 2 (TMPRSS2) gene, TMPRSS2 pre-mRNA or TMPRSS2 mRNA, and in some embodiments, the nucleic acid base sequence is antisense to at least a portion of the TMPRSS2 pre-mRNA or TMPRSS2 mRNA. In any embodiments, the nucleic acid base sequence may comprise from 2 to 50 nucleic acid bases, such as from 20 to 30 nucleic acid bases. In some embodiments, the oligomer's nucleic acid base sequence is 5′-CAGAGTTGGAGCACTTGCTGCCCA-3′ (SEQ ID NO: 2).

The oligomer may further comprise a steric blocking backbone on which the nucleic acid bases are attached. The backbone may comprise, consist essentially of, or consist of, phosphorodiamidate morpholino (PMO), methylphosphonate, 2′-O-methyl RNA (2′-)Me), 2′-O-methyl phosphorothioate (2′-OMePS), 2′-O-methoxyethyl RNA (2′-MOE), 2′-O-methoxyethyl phosphorothioate (2′-MOE-PS), peptide nucleic acid (PNA), tricycle-DNA (tcDNA), locked nucleic acid (LNA), or combinations thereof, and/or may have a structure

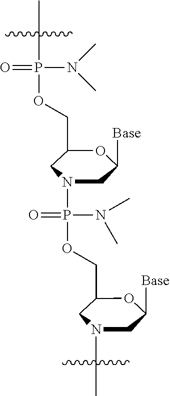

where each Base refers to the attachment point of a nucleic acid base.

The compound may further comprise a peptide covalently attached to the oligomer. The peptide may be attached to the 3′ end or the 5′ end of the oligomer. In some embodiments, the peptide comprises from 2 to 60 amino acids, such as from 2 to 40 amino acids, from 5 to 30 amino acids or from 10 to 15 amino acids. Each amino acid independently may be selected from the L and/or D form of glycine, valine, alanine, leucine, isoleucine, methionine, phenylalanine, tryptophan, tyrosine, serine, threonine, asparagine, glutamine, arginine, histidine, lysine, aspartic acid, glutamic acid, cysteine, proline, beta-alanine, selenocysteine, pyrrolysine, 7-aminoheptanoic acid, 6-amino hexanoic acid, 5-aminopentanoic acid, 4-aminobutanoic acid, or homoarginine. And in some embodiments, the peptide is selected to facilitate transport of the compound upon administration. In some embodiments, the peptide is RAhxRRAhxRRAhxRRAhxRAhxB where R=Arginine, Ahx=6-aminohexanoic acid, and B=beta-alanine (SEQ ID NO: 59).

The method may comprise administering the composition by any suitable route, such as inhalation. Additionally, or alternatively, the method may comprise administering from 0.01 mg/kg to about 30 mg/kg of the compound, such as from 0.01 mg/kg to about 10 mg/kg. And in some embodiments, the compound is administered 1, 2, 3, 4 or more times a day.

In a particular embodiment, a method for treating or preventing a SARS-CoV-2 infection in a human subject comprises administering to the subject an effective amount of a compound having a structure

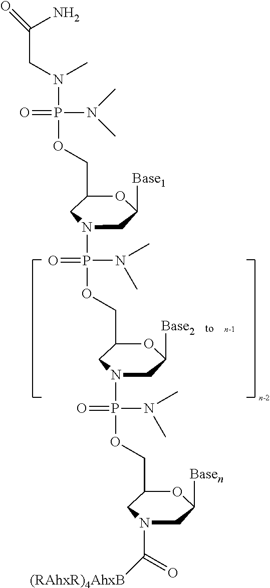

or a pharmaceutically acceptable salt thereof, wherein n is from 20 to 30; each Base independently is selected from adenine, guanine, cytosine or thymine; R is Arginine; Ahx is 6-aminohexanoic acid; and B is beta-alanine. And in certain embodiments, n is 24 and the nucleic acid base sequence from Base₁ to Base₂₄ is CAGAGTTGGAGCACTTGCTGCCCA (SEQ ID NO: 2), resulting in compound T-ex5.

The foregoing and other objects, features, and advantages of the invention will become more apparent from the following detailed description, which proceeds with reference to the accompanying figures.

### BRIEF DESCRIPTION OF THE DRAWINGS

FIG. 1 is a table providing exemplary nucleic acid base sequences suitable for use in the disclosed compounds, and illustrating potential regions of transmembrane serine protease 2 (TMPRSS2) mRNA or pre-mRNA that may be targeted by the sequences.

FIG. 2 provides an exemplary general formula for a compounds suitable for use in the disclosed method, and illustrates the structural features of a peptide phosphorodiamidate morpholino oligomers (PPMO).

FIG. 3 is a digital image illustrating the strong cytopathic effect (CPE) and efficient spreading of SARS-CoV-2 infection in untreated Calu-3-cells.

FIG. 4 is a digital image illustrating the strong CPE and efficient spreading of SARS-CoV-2 infection in Calu-3-cells treated with a nonsense sequence designated “scramble.”

FIG. 5 is a digital image illustrating the absence of CPE and significantly reduced SARS-CoV-2 spreading in Calu-3-cells treated with T-ex5.

FIG. 6 is a graph of TCID₅₀ versus time post infection, illustrating the results from the second experiment examining the effect of T-ex5 administration on SARS-CoV-2 activation and multicycle replication.

FIG. 7 provides digital images illustrating the TMPRSS2-specific mRNA identified by PCR from Calu-3 cells. FIG. 7A provides the results from cells treated with 25 μM T-ex5 or PPMO scramble and compared to the mRNA from untreated cells (w/o) and illustrates that only a truncated PCR fragment lacking exon 5 was amplified from T-ex5 treated cells. And FIG. 7B provides the results 3 at 72 hours post infection from Calu-3 cells that were inoculated with SARS-CoV-, and illustrates that in infected cells, only the truncated PCR fragment lacking exon 5 was amplified from T-ex5 treated cells.

FIG. 8 is a graph of cell viability versus treatment, illustrating that T-ex5 treatment does not negatively affect cell viability.

FIG. 9 provides structures of exemplary oligomer backbone structures with steric-blocking moieties that resist cleavage when administered to a subject.

FIG. 10 provides alternative PMO structures suitable for use in the disclosed compounds.

### SEQUENCE LISTING

The nucleic and amino acid sequences listed in the accompanying sequence listing are shown using standard letter abbreviations for nucleotide bases, and single letter code for amino acids, as defined in 87 Fed. Reg. 30806. Only one strand of each nucleic acid sequence is shown, but the complementary strand is understood as included by any reference to the displayed strand. The Sequence Listing is submitted as an Extensible Markup Language (XML) file, created on Oct. 10, 2022 (1,314,816 bytes) which is incorporated by reference herein.

SEQ ID NO: 1 is the gene sequence for the human transmembrane serine protease 2 (TMPRSS2) gene (GenBank No. NG 047085).

SEQ ID NOs: 2-30 are exemplary nucleic acid base sequences suitable for use in the disclosed compounds.

SEQ ID NOs: 31-58 are potential pre-mRNA or mRNA regions targeted by SEQ ID NOs: 3-30.

SEQ ID NOs: 59-539 are exemplary peptide sequences suitable for use in the disclosed compounds.

SEQ ID NO: 540 is an exemplary random nucleic acid base sequence (referred to herein as “scramble”) suitable for use as a control.

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

When chemical structures are depicted or described, unless explicitly stated otherwise, all carbons are assumed to include implicit hydrogens such that each carbon conforms to a valence of four. For example, in the structure on the left-hand side of the schematic below there are nine hydrogen atoms implied. The nine hydrogen atoms are depicted in the right-hand structure.

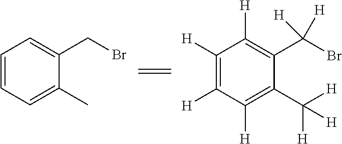

Sometimes a particular atom in a structure is described in textual formula as having a hydrogen or hydrogen atoms, for example —CH₂CH₂—. It will be understood by a person of ordinary skill in the art that the aforementioned descriptive techniques are common in the chemical arts to provide brevity and simplicity to description of organic structures.

### II. Overview

Coronaviruses (CoV) are a large group of enveloped, single-stranded positive-sense RNA viruses belonging to the order Nidovirales that infect a broad range of mammalian and avian species, typically causing respiratory and/or enteric tract disease. CoV infection is initiated by the transmembrane spike (S) glycoprotein present as a large trimer at the surface of the virion through binding to the host cell receptor and fusion of the viral and cellular membranes. The CoV S protein requires cleavage by host cell proteases to undergo conformational changes that mediate virus entry and membrane fusion.

In 2006, the transmembrane serine protease 2 (TMPRSS2) was identified as a human protease present in the airways that is capable of activating the surface glycoprotein hemagglutinin of human influenza A viruses. Disclosed herein is data demonstrating that TMPRSS2 also is implicated in SARS-CoV-2 activation in human airway cells. The data also demonstrate that TMPRSS2 inhibition is sufficient to strongly suppress virus multiplication and spread in Calu-3 human airway epithelial cells.

### III. Compounds

Disclosed herein are embodiments of steric-blocking antisense oligomers useful for treating and/or preventing SARS-CoV-2 infections. The compound may comprise one or more oligomers that comprise a nucleic acid base sequence that is antisense to DNA and/or RNA that is implicated in SARS-CoV-2 infection and/or replication. In some embodiments, the oligomer may comprise a nucleic acid base sequence that is antisense to a nucleic acid base sequence from a gene, pre-mRNA, and/or mRNA that is present in the infected subject. The oligomer's nucleic acid base sequence may comprise, consist essentially of, or consist of from 2 to 50 or more bases, from 5 to 50 bases, from 10 to 40 bases, from 10 to 30 bases, from 15 to 30 bases or from 20 to 30 bases, and in some embodiments, the oligomer comprises a sequence of 24 bases. In some embodiments, the compound comprises an oligomer that is antisense to a TMPRSS2 gene such as the human TMPRSS2 gene, or to pre-mRNA and/or mRNA that is transcribed from the TMPRSS2 gene. SEQ ID No: 1 provides an exemplary human TMPRSS2 gene, and a person of ordinary skill in the art understands that the pre-mRNA is a primary transcript that is synthesized from the gene DNA.

The oligomer(s) may further comprise a backbone that comprises bonds and/or structural moieties that are resistant to degradation when administered to a subject and/or exposed to typical cellular DNA and/or RNA cleavage mechanisms, such as mechanisms suitable to cleave the phosphate linkages in DNA or RNA. In some embodiments, moieties on the backbone sterically block DNA and/or RNA cleavage mechanisms. Suitable backbones include, but are not limited to, a phosphorodiamidate morpholino (PMO) backbone, methylphosphonate, 2′-O-methyl RNA, peptide nucleic acid (PNA), or locked nucleic acid (LNA), as illustrated below.

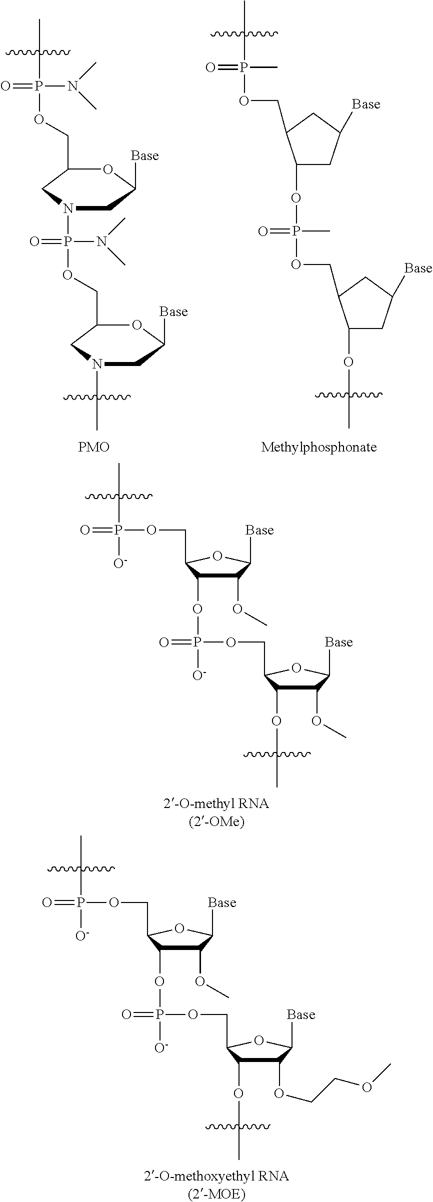

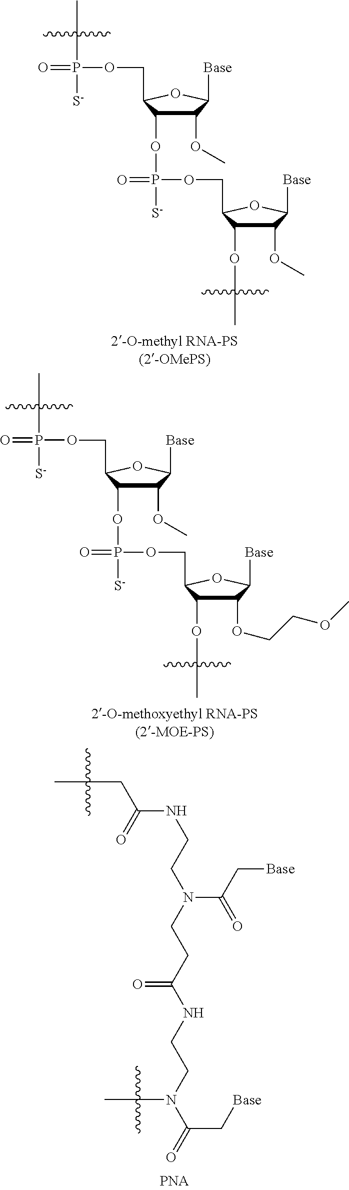

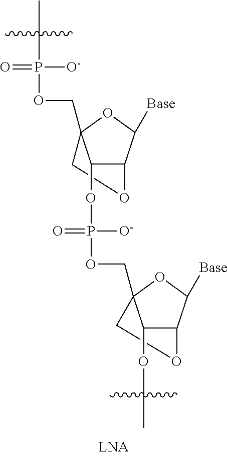

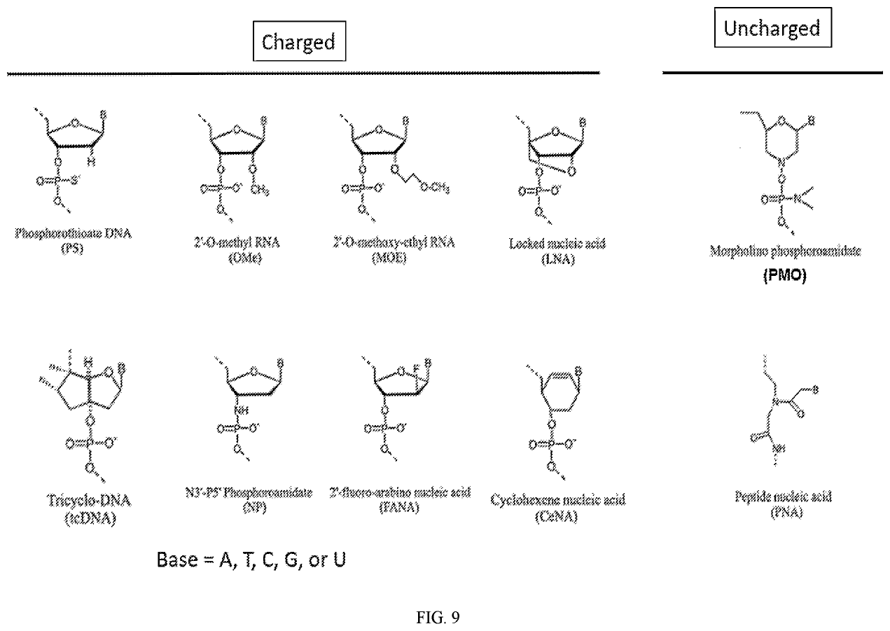

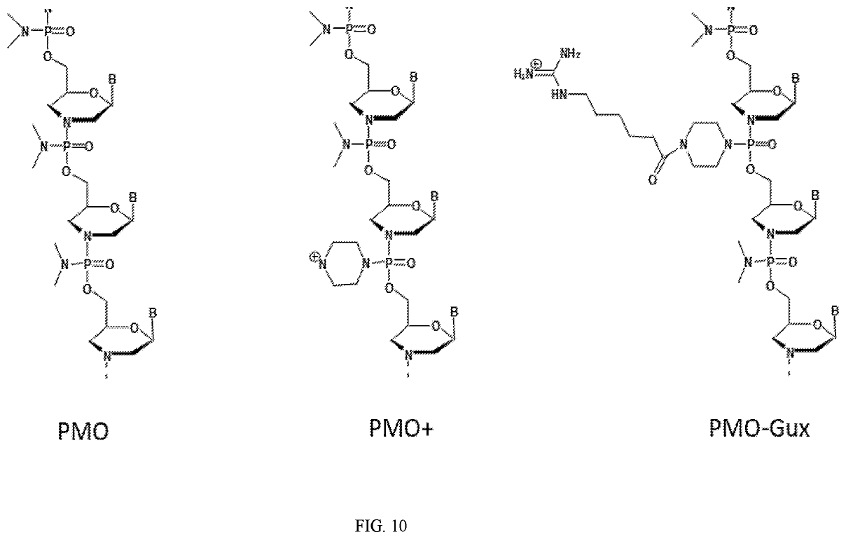

FIGS. 9 and 10 provide additional exemplary backbone moieties suitable for use in the disclosed compounds. FIG. 9 provides exemplary monomer units suitable for use in the backbone structure of the disclosed compound. FIG. 10 provides examples of modified PMO structures, such as charged structures comprising one or more piperazine moieties that optionally can be substituted, such as with an amino acid. A person of ordinary skill in the art understands that the nucleic acid backbone of the disclosed compound may comprise, consist essentially of, or consist of, one of the monomer unit types disclosed herein, or it may comprise, consist essentially of, or consist of, more than one type of monomer unit, such as 2, 3, 4, 5, 6, or more monomer unit types.

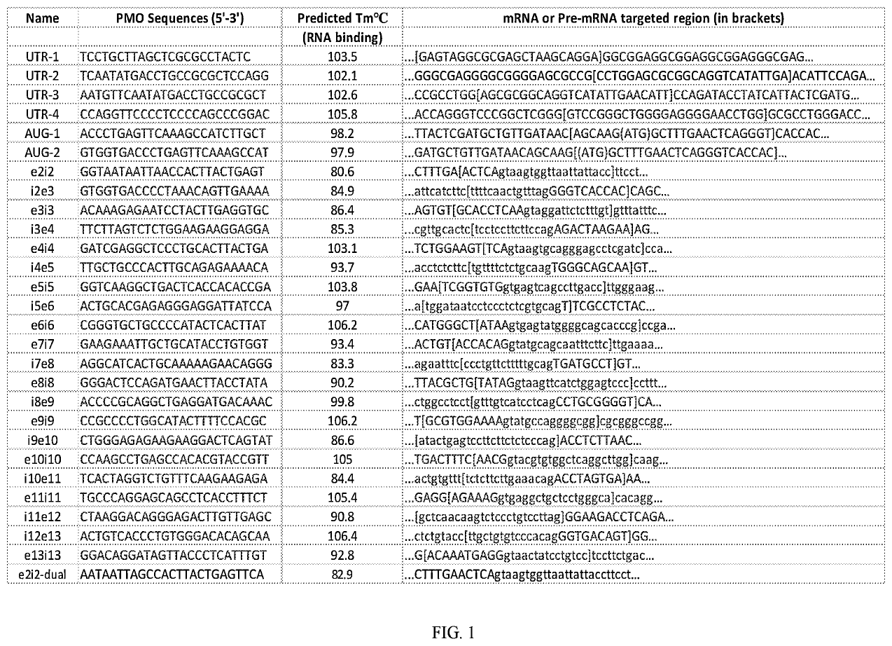

Certain exemplary nucleic acid base sequences suitable for use in the disclosed compounds are provided in FIG. 1 and/or include, but are not limited to, the following: (all sequences shown from 5′ to 3′)

(SEQ ID NO: 2)
CAGAGTTGGAGCACTTGCTGCCCA;
(SEQ ID NO: 3)
TCCTGCTTAGCTCGCGCCTACTC;
(SEQ ID NO: 4)
TCAATATGACCTGCCGCGCTCCAGG;
(SEQ ID NO: 5)
AATGTTCAATATGACCTGCCGCGCT;
(SEQ ID NO: 6)
CCAGGTTCCCCTCCCCAGCCCGGAC;
(SEQ ID NO: 7)
ACCCTGAGTTCAAAGCCATCTTGCT;
(SEQ ID NO: 8)
GTGGTGACCCTGAGTTCAAAGCCAT;
(SEQ ID NO: 9)
GGTAATAATTAACCACTTACTGAGT;
(SEQ ID NO: 10)
GTGGTGACCCCTAAACAGTTGAAAA;
(SEQ ID NO: 11)
ACAAAGAGAATCCTACTTGAGGTGC;
(SEQ ID NO: 12)
TTCTTAGTCTCTGGAAGAAGGAGGA;
(SEQ ID NO: 13)
GATCGAGGCTCCCTGCACTTACTGA;
(SEQ ID NO: 14)
TTGCTGCCCACTTGCAGAGAAAACA;
(SEQ ID NO: 15)
GGTCAAGGCTGACTCACCACACCGA;
(SEQ ID NO: 16)
ACTGCACGAGAGGGAGGATTATCCA;
(SEQ ID NO: 17)
CGGGTGCTGCCCCATACTCACTTAT;
(SEQ ID NO: 18)
GAAGAAATTGCTGCATACCTGTGGT;
(SEQ ID NO: 19)
AGGCATCACTGCAAAAAGAACAGGG;
(SEQ ID NO: 20)
GGGACTCCAGATGAACTTACCTATA;
(SEQ ID NO: 21)
ACCCCGCAGGCTGAGGATGACAAAC;
(SEQ ID NO: 22)
CCGCCCCTGGCATACTTTTCCACGC;
(SEQ ID NO: 23)
CTGGGAGAGAAGAAGGACTCAGTAT;
(SEQ ID NO: 24)
CCAAGCCTGAGCCACACGTACCGTT;
(SEQ ID NO: 25)
TCACTAGGTCTGTTTCAAGAAGAGA;
(SEQ ID NO: 26)
TGCCCAGGAGCAGCCTCACCTTTCT;
(SEQ ID NO: 27)
CTAAGGACAGGGAGACTTGTTGAGC;
(SEQ ID NO: 28)
ACTGTCACCCTGTGGGACACAGCAA;
(SEQ ID NO: 29)
GGACAGGATAGTTACCCTCATTTGT;
or
(SEQ ID NO: 30)
AATAATTAGCCACTTACTGAGTTCA.

A person of ordinary skill in the art understands that with respect to the sequences disclosed herein, A, G, C, T and U represent bases adenine, guanine, cytosine, thymine and uracil, respectively, as shown below, where the wavy line indicates the point of attachment to the oligomer backbone.

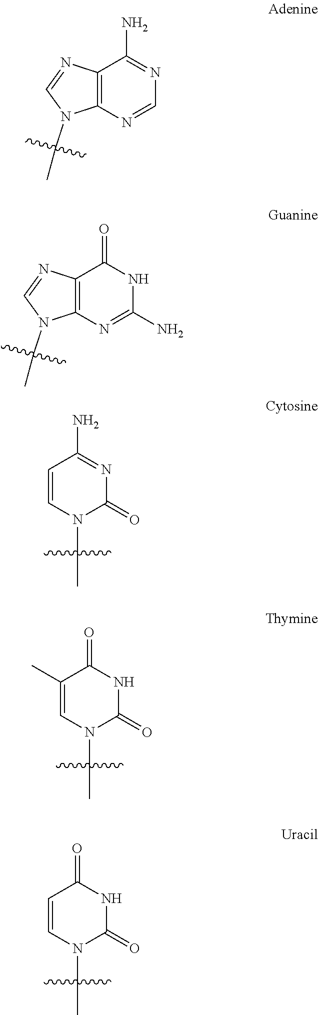

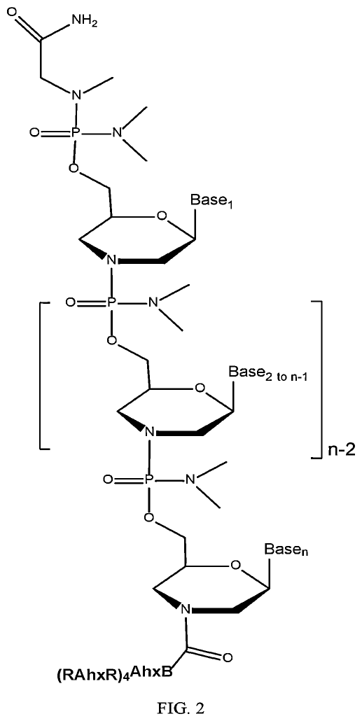

In some embodiments, the compound further comprises a peptide sequence covalently attached to the oligomer, and the compound may have a formula: Peptide-Oligomer, Peptide-Oligomer-Peptide, Peptide1-Oligomer-Peptide2, or Peptide1-Peptide2-Oligomer, where Peptide1 and Peptide2 have different amino acid sequences. Additionally, a peptide can be in either linear or branched form. In certain embodiments, the oligomer comprises a PMO backbone, and the compound may be a peptide-conjugated PMO (PPMO). The peptide may be selected and/or designed to facilitate transport of the compound, such as through a membrane and/or into a cell. The peptide may be a naturally occurring sequence, such as a protein or fragment thereof, or the peptide may be a non-naturally occurring amino acid sequence. FIG. 2 provides an exemplary chemical structure of a PPMO. With respect to the example in FIG. 2, n is the number of nucleic acid bases in the compound, R is arginine, Ahx is 6-aminohexanoic acid, B is beta-alanine, and each Base indicates a nucleic acid base. Also with respect to FIG. 2, when n is 24 and Base₁ to Base₂₄ is CAGAGTTGGAGCACTTGCTGCCCA (SEQ ID NO: 2), the compound is T-ex5.

However, a person of ordinary skill in the art understands that the suitable peptides may comprise any amino acid, such as one or more of natural amino acids, such as glycine, valine, alanine, leucine, isoleucine, methionine, phenylalanine, tryptophan, tyrosine, serine, threonine, asparagine, glutamine, arginine, histidine, lysine, aspartic acid, glutamic acid, cysteine, or proline, and such amino acids may be the L-amino acid, the D-amino acid or a mixture thereof. In some embodiments, a natural amino acid in the peptide is the L-amino acid. Additionally, or alternatively, the peptide may comprise one or more alternative naturally occurring or non-naturally occurring amino acids, for example, beta-alanine, selenocysteine, pyrrolysine, 7-aminoheptanoic acid, 6-amino hexanoic acid, 5-aminopentanoic acid, 4-aminobutanoic acid, or homoarginine. The peptide may be of any length suitable to facilitate transport of the compound. In some embodiments, the peptide comprises, consists essentially of, or consists of, from 2 amino acid to 60 amino acids or more, such as from 2 amino acid to 40 amino acids, from 5 to 30 amino acids, from 5 to 20 amino acids, from 10 to 20 amino acids or from 10 to 15 amino acids. In certain disclosed embodiments, the peptide has a length of 14 amino acids.

The peptide may be attached to the oligomer via the oligomer backbone, and may be attached at the 3′ end of the oligomer, such as in FIG. 2, or it may be attached to the 5′ end of the oligomer. Additionally, the peptide may be attached to the oligomer by any suitable bond, such as an amide bond (as shown in FIG. 2), maleimide bond, a disulfide bond, an ester bond, or a bond formed by “click” chemistry with or without being catalyzed by copper ions.

Exemplary peptides useful in the disclosed technology include, but are not limited to, the exemplary protein sequences provided by SEQ ID NOs: 59-539. In particular embodiments, the peptide is RAhxRRAhxRRAhxRRAhxRAhxB where R=Arginine, Ahx=6-aminohexanoic acid, and B=beta-alanine (SEQ ID NO: 59).

In some embodiments, the compound has a structure according to Formula 1

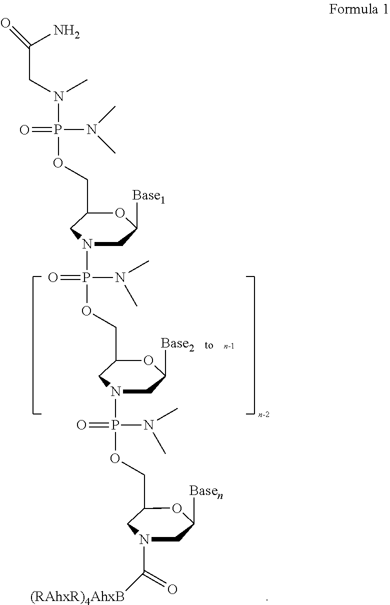

With respect to Formula 1, n is from 2 to 50, such as from 5 to 50, from 10 to 40, from 15 to 30 or from 20 to 30, and in certain embodiments, n is 24.

Each base independently is selected from adenine, guanine, cytosine, thymine or uracil, and may be selected from adenine, guanine, cytosine or thymine.

R is Arginine, Ahx is 6-aminohexanoic acid, and B is beta-alanine.

In one exemplary embodiment of Formula 1, n is 24, and nucleic acid base sequence from Base₁ to Base₂₄ is CAGAGTTGGAGCACTTGCTGCCCA (SEQ ID NO: 2), resulting in compound T-ex5.

### IV. Method for Administering the Compounds

A. Formulation and Administration

The disclosed compounds described herein can be formulated as pharmaceutical compositions and administered to a mammalian host, such as a human or veterinary patient, in a variety of forms. The forms can be specifically adapted to a chosen route of administration, e.g., oral or parenteral administration, by intravenous, intramuscular, inhalation, such as intranasal, or subcutaneous routes.

For nasal administration or administration by inhalation or insufflation, the active compound, and/or an pharmaceutically acceptable salt, can be conveniently delivered in the form of an aerosol spray from pressurized packs or a nebulizer with the use of a suitable propellant, for example, dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch. The compound may be dissolved in water or other suitable aqueous solution and aerosolized for inhalation. Alternatively, the compound may be provided as a dry powder suitable for inhalation.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 2% to about 60% of the weight of a given unit dosage form. The amount of active compound in such therapeutically useful compositions is such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, methods of preparation can include vacuum drying and freeze drying techniques, which yield a powder of the active ingredient plus any additional desired ingredient present in the previously sterile-filtered solutions.

For any route of administration, the compounds described herein can be used to prepare therapeutic pharmaceutical compositions. In some embodiments, the compounds are soluble in water or dilute saline solution, such as an isotonic or less than isotonic saline solution. In other embodiments, the compounds may be added to the compositions in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartrate, succinate, benzoate, ascorbate, α-ketoglutarate, and b-glycerophosphate. Suitable inorganic salts may also be formed, including hydrochloride, halide, sulfate, nitrate, bicarbonate, and carbonate salts.

Pharmaceutically acceptable salts may be obtained using procedures known to persons of ordinary skill in the art, for example by reacting a sufficiently basic compound, such as an amine, with a suitable acid to provide a physiologically acceptable ionic compound. Alkali metal (for example, sodium, potassium or lithium) or alkaline earth metal (for example, calcium) salts of carboxylic acids can also be prepared by analogous methods.

B. Dosage

The disclosed compounds, pharmaceutical compositions and/or combination s thereof will generally be used in an affective amount to treat and/or prevent SARS-CoV-2 infection in a subject, such as a human or non-human animal, particularly a mammal. The disclosed compound(s), or pharmaceutical compositions thereof, can be administered therapeutically to achieve therapeutic benefit or prophylactically to achieve a prophylactic benefit. Therapeutic benefit means amelioration or eradication of a SARS-CoV-2 infection and/or an improvement, such as an easing or ceasing, of one or more symptoms associated with a SARS-CoV-2 infection, such that the subject experiences and/or reports an improvement in feeling or condition, even if the subject is still infected with the SARS-CoV-2 virus. Symptoms of SARS-CoV-2 that may be improved by administering one or more of the disclosed compounds include, but are not limited to, a fever, cough, such as a dry cough, difficulty breathing, shortness of breath, muscle or body aches, pain or pressure in the chest, fatigue, nasal congestion and/or sore throat. Therapeutic benefit also includes halting or slowing the progression of disease caused by SARS-CoV-2, regardless of whether improvement is realized.

A person of ordinary skill in the art understands that a preferred dosage of one or more of the disclosed compounds may depend on various factors, including the age, weight, general health, and severity of the condition of the subject being treated. Dosage also may need to be tailored to the sex of the individual and/or the lung capacity of the individual, when administered by inhalation. Additionally, dosages may be individually tailored for subjects having an underlying condition in addition to SARS-CoV-2, and/or subjects who have additional conditions that affect lung capacity and/or the ability to breath normally. Underlying conditions may include, but are not limited to, blood disorders, such as sickle cell disease or taking blood thinners; chronic kidney or liver disease; conditions that weaken the immune system, such as cancer or cancer treatment, organ or bone marrow transplant, immunosuppressant medications, HIV or AIDS; current or recent pregnancy in the last two weeks; diabetes; inherited metabolic disorders and mitochondrial disorders; heart disease, including coronary artery disease, congenital heart disease, and heart failure; lung disease, including asthma, or COPD; neurological and neurologic and neurodevelopment conditions such as cerebral palsy, epilepsy (seizure disorders), stroke, muscular dystrophy, or spinal cord injury; or a combination thereof. Dosage and frequency of administration of the disclosed compound(s) or pharmaceutical compositions thereof, also will depend on whether the disclosed compound(s) are formulated and/or administered for treatment of a SARS-CoV-2 infection, or are formulated and/or administered prophylactically to prevent a SARS-CoV-2 infection. A person of ordinary skill in the art will be able to determine the optimal dose for a particular individual.

For prophylactic administration, the disclosed compound(s), or pharmaceutical compositions thereof, can be administered to a subject at risk of being infected by the SARS-CoV-2 virus. For example, if a subject works in the medical field with patients suffering from SARS-CoV-2 infections, the disclosed compound(s), or a pharmaceutical composition thereof, may be administered to help prevent the subject from becoming infected. Additionally, or alternatively, the disclosed compound(s), or pharmaceutical compositions thereof, may be administered to a subject having one or more underlying conditions that may make them more at risk of developing serious disease from a SARS-CoV-2 infection, such as one or more of the underlying conditions listed herein.

Effective dosages can be estimated initially from in vitro assays. For example, an initial dosage for use in subjects can be formulated to achieve a circulating blood or serum concentration of active compound that is at or above an IC₅₀ or EC₅₀ of the particular compound as measured in an in vitro assay. Dosages can be calculated to achieve such circulating blood or serum concentrations taking into account the bioavailability of the particular compound. Fingl & Woodbury, “General Principles,” In: Goodman and Gilman's The Pharmaceutical Basis of Therapeutics, Chapter 1, pages 1-46, Pergamon Press, and the references cited therein, provide additional guidance concerning effective dosages.

Initial dosages can also be estimated from in vivo data, such as animal models. For dosage estimation for human administration, suitable animal models may either be animals selected or genetically modified to be susceptible to infection by human strains of SARS-CoV-2, or dosages can be estimated from administration to animals infected with a suitable animal analog of SARS-CoV-2. Persons of ordinary skill in the art can adapt such information to determine dosages suitable for human administration.

Dosage amounts of disclosed compounds will typically be in the range of from greater than 0 mg/kg/day, such as 0.0001 mg/kg/day or 0.001 mg/kg/day or 0.01 mg/kg/day, up to at least about 100 mg/kg/day. More typically, the dosage (or effective amount) may range from about 0.0025 mg/kg to about 50 mg/kg administered at least once per day, such as from 0.01 mg/kg to about 30 mg/kg, from 0.01 mg/kg to about 20 mg/kg, from 0.01 mg/kg to about 10 mg/kg, or from about 0.05 mg/kg to about 5 mg/kg. The total daily dosage typically ranges from about 0.1 mg/kg to about 100 mg/kg or to about 30 mg/kg per day, such as from 0.5 mg/kg to about 20 mg/kg per day, or from 0.5 mg/kg to about 10 mg/kg per day. Dosage amounts can be higher or lower depending upon, among other factors, the activity of the disclosed compound, its bioavailability, the mode of administration, and various factors discussed above.

Dosage amount and dosage interval can be adjusted for subjects to maintain a therapeutic or prophylactic effect. For example, the compounds can be administered once per day, multiple times per day, such as 2, 3, 4 or more time per day, once per week, multiple times per week (for example, 2, 3, 4, 5, 6, or 7 times a week, or every other day), one per month, multiple times per month (for example, 2, 3, 4, 5, 6, 7, 8, 9, 10 or more times a month), or once per year, depending upon, amongst other things, the mode of administration, the severity of symptoms with respect to a therapeutic administration, the likelihood of infection with respect to prophylactic administration, and the judgment of the prescribing physician. Persons of ordinary skill in the art will be able to optimize effective local dosages without undue experimentation.

Preferably, the disclosed compound, combinations of disclosed compounds, or pharmaceutical compositions thereof, will provide therapeutic or prophylactic benefit without causing substantial toxicity to a subject. Toxicity of the disclosed compound can be determined using standard pharmaceutical procedures known to persons of ordinary skill in the art. The dose ratio between toxic and therapeutic (or prophylactic) effect is the therapeutic index. Disclosed compounds that exhibit high therapeutic indices are preferred.

C. Additional Therapies

The disclosed compound(s), or pharmaceutical compositions thereof, may be administered alone or in combination with one or more additional therapies. Suitable additional therapies include any therapy that may be administered to treat an underlying condition, to ameliorate one or more symptoms of SARS-CoV-2 infection, and/or to treat or prevent a SARS-CoV-2 infection. In some embodiments, the disclosed compound(s), or pharmaceutical compositions thereof, are administered in combination with, but are not limited to, an antibiotic, anti-inflammatory agent (such as a steroidal anti-inflammatory agent or a nonsteroidal anti-inflammatory agent), analgesic, antiviral, antibody, or a combination thereof. Exemplary analgesics include, but are not limited to, morpholine, hydromorphone, oxycodone, codeine, acetaminophen, hydrocodone, buprenorphine, tramadol, fentanyl, meperidine, pentazocine, or combinations thereof. Exemplary antibiotics include, but are not limited to, penicillins, aminoglycosides, quinolones, cephalosporins, tetracyclines, sulfonamides, macrolides, nitrofurans, or combinations thereof. Exemplary anti-inflammatory agents include, but are not limited to, budesonide, aminosalicylates, cyclooxygenase inhibitors, ibuprofen, naproxen, ketoprofen, or a combination thereof.

### V. Examples

### Materials and Methods

Cells and Virus. Calu-3 human airway epithelial cells (ATCC® HTB55) were cultured in Dulbecco's modified Eagle's medium (DMEM)-Ham F-12 medium (1:1) (Gibco) supplemented with 10% fetal calf serum (FCS), penicillin, streptomycin, and glutamine, with fresh culture medium replenished every 2 to 3 days.
Virus and plasmids. Experiments with SARS-CoV-2 were performed under biosafety level 3 (BSL-3) conditions. The virus used in this study was SARS-CoV-2 isolate Munich 929 (kindly provided by Christian Drosten, Charité, Berlin, Germany). Virus stock was propagated on Vero E6 cells in infection medium. Cell supernatant was cleared by low-speed centrifugation and stored at −80° C.
PPMO. Phosphorodiamidate morpholino oligomers (PMO) were synthesized at Gene Tools LLC (Corvallis, Oreg., USA). PMO sequences (5′ to 3′) were CAGAGTTGGAGCACTTGCTGCCCA (SEQ ID NO: 2) for T-ex5 and CCTCTTACCTCAGTTACAATTTATA (SEQ ID NO: 540) for scramble. The cell-penetrating peptide (RXR)4 (where R is arginine and X is 6-aminohexanoic acid) was covalently conjugated to the 3′ end of each PMO through a noncleavable linker, to produce peptide-PMO (PPMO), by methods described herein.

### Example 1

### Synthesis of T-ex5 PPMO

The delivery peptide (RAhxRRAhxRRAhxRRAhxRAhxB, R=Arginine, Ahx=6-aminohexanoic acid, B=beta-alanine; SEQ ID NO. 59) and the T-ex5 PMO were purchased from a peptide supplier and Gene Tools LLC (Philomath, Oreg.), respectively. For conjugation of the peptide to the PMO, the PMO was dissolved in dimethylsufoxide (DMSO) at about 100 mg/mL. The peptide solution was made by dissolving peptide powder in DMSO (100 mg/mL). The peptide solution (1 eq) was activated by first adding HBTU (1 eq) and followed by adding N,N-diisopropylethylamine (DIEA) (1 eq). Immediately after the addition of DIEA, the peptide solution was mixed and added to the PMO solution at a peptide to PMO reaction ratio of 1.5 to 1. After 2 hours at 45° C., the reaction mixture was diluted with a threefold excess of water. The crude conjugate was purified by strong cation exchange liquid chromatography using a Tricorn Source 15s HPLC column (GE Healthcare, Piscataway, N.J.). Elution of the sample was carried out via a linear NaCl gradient in a 20 mM pH=7 sodium phosphate buffer containing 25% (v:v) acetonitrile. The desired fractions were pooled, desalted by a solid phase extraction method and analyzed by HPLC and mass spectrometry. The product was then quantified and lyophilized.

### Example 2

### RNA Isolation, RT-PCR Analysis of Exon Skipping, and RT-qPCR Analysis of Protease Transcripts

For analysis of TMPRSS2-mRNA from PPMO-treated Calu-3 cells, cells were incubated with the indicated concentrations (2504) of T-ex5 or scramble PPMO or without PPMO in PPMO medium (DMEM supplemented with 0.1% BSA, antibiotics, and glutamine) for 24 hours. Total RNA was isolated at the indicated time points using the RNeasy Mini kit (QIAGEN) according to the manufacturer's protocol. Reverse transcription-PCR (RT-PCR) was carried out with total RNA using the one-step RT-PCR kit (QIAGEN) according to the supplier's protocol. To analyze TMPRSS2 mRNAs for exon skipping, primers TMPRSS2-108fwd (5′-CTA CGA GGT GCA TCC-3′) and TMPRSS2-1336rev (5′-CCA GAG GCC CTC CAG CGT CAC CCT GGC AA-3′), designed to amplify a full-length PCR product of 1,228 bp from control cells and a shorter PCR fragment of about 1,100 bp (Aex5) from T-ex5-treated cells were used. RT-PCR products were resolved on a 0.8% agarose gel stained with ethidium bromide.

### Example 3

### Infection of Cells and Multicycle Replication in the Presence of PPMO

Infection experiments of Calu-3 cells were performed in serum-free DMEM supplemented with glutamine and antibiotics (DMEM++). For analysis of multicycle replication kinetics Calu-3 cells were seeded in 12-well plates and grown to 90% confluence. For PPMO treatment, Calu-3 cells were incubated with 25 μM T-ex5 or scramble PPMO or remained untreated in PPMO medium for 24 hours prior to infection. Cells were then inoculated with virus at an MOI of 0.001 in DMEM++ for 1.5 hours, washed with PBS, and incubated in DMEM supplemented with 3% FCS, glutamine and antibiotics (DMEM++/3% FCS) without addition of T-ex5 PPMO to the medium for 72 hours. At 16, 24, 48, and 72 hours post infection, supernatants were collected, and viral titers were determined by tissue culture infection dose 50% (TCID₅₀) titration as described below. In addition, cells were fixed and immunostained against viral proteins as described below at 72 hours post infection to evaluate virus spread and virus-induced CPE.

### Example 4

### Virus Titration by TCID₅₀

Viral supernatants were serial diluted in DMEM++. Each infection time point was titrated in 4 replicates from 5¹ to 5¹¹. Subsequently, 100 μl of each virus dilution were transferred to Calu-3 cells grown in 96-well cell culture plates containing 100 μl 3 DMEM and incubated for 72 hours. Viral titers were determined with Spearman and Kärber algorithm. This experiment was repeated several times with similar results.

### Example 5

### Immunohistochemical Staining and Microscopy

To visualize viral spread in SARS-CoV-2-infected Calu-3 cells, immunohistochemical staining was performed. Calu-3 cells were fixed 72 hours post infection in 4% paraformaldehyde (PFA) for 36 hours at 4° C. The cells were permeabilized with 0.3% triton-X-100 (Sigma Aldrich) for 20 minutes at room temperature (RT). Subsequently, the cells were incubated with a polyclonal rabbit serum against SARS-CoV (Institute of Virology, Marburg, Germany) for 1.5 hours at RT, then washed three times with PBS. After washing the cells were incubated with a species-specific peroxidase-conjugated secondary antibody for 1 hour at RT. The immunohistological staining was visualized using the peroxidase substrate KPL TrueBlue™ (Seracare) and further analyzed on a Leica Dmi1 microscope.

### Example 6

### Cell Viability Assay

Cell viability was assessed by measuring the cellular ATP content using the CellTiterGlo® luminescent cell viability assay (Promega). Calu-3 cells grown in 96-well plates were incubated with 25 μM of each PPMO for 24 hours. Subsequently, cells were incubated with the substrate according to the manufacturer's protocol. Luminescence was measured using a black 96-well plate (Nunc) with a luminometer (Centro LB 960; Berthold Technologies). The absorbance values of PPMO-treated cells were converted to percentages by comparison to untreated control cells, which were set at 100% cell viability.

### Results

### Knockdown of TMPRSS2 Prevents Proteolytic Activation and Multiplication of SARS-CoV-2 in Calu-3 Human Airway Epithelial Cells

T-ex5, an antisense peptide-conjugated phosphorodiamidate morpholino oligomer (PPMO), was used to investigate whether TMPRSS2 is involved in proteolytic activation and multicycle replication of SARS-CoV-2 S in Calu-3 human airway epithelial cells. PPMO are single-stranded nucleic-acid-like antisense agents composed of a morpholino oligomer covalently conjugated to a cell-penetrating peptide, and can interfere with gene expression by sterically blocking complementary RNAs. PPMO T-ex5 interferes with correct splicing of TMPRSS2 pre-mRNA, resulting in the production of mature mRNA lacking exon 5 and consequently expression of a truncated TMPRSS2 form that is enzymatically inactive.

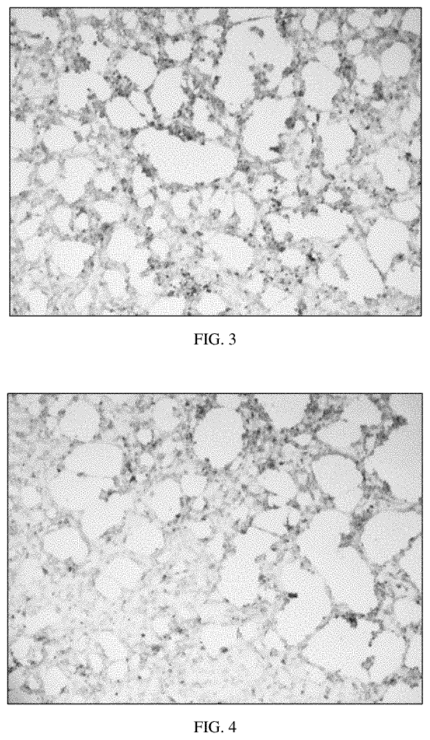

T-ex5 PPMO-mediated knockdown of TMPRSS2 activity previously identified TMPRSS2 as the major influenza A virus activating protease in Calu-3 cells and primary human airway epithelial cells and of influenza B virus in primary human type II pneumocytes. Here, Calu-3 cells were treated once with 25 μM T-ex5 PPMO for 24 hours prior to infection with SARS-CoV-2, in order to inhibit the production of normal TMPRSS2-mRNA and deplete enzymatically active TMPRSS2 present in the cells. The cells were then inoculated with SARS-CoV-2 at a low multiplicity of infection (MOI) of 0.001 and further incubated without additional PPMO treatment for 72 hours. Then, cells were fixed and immunostained using an antiserum against the 2002 SARS-CoV. As shown in FIGS. 3 and 4, strong cytopathic effect (CPE) and efficient spread of SARS-CoV-2 infection was visible in the control cells comprising untreated Calu-3 cells (FIG. 3) and Calu-3 cells treated with a negative-control PPMO of nonsense sequence designated “scramble” (FIG. 4). In contrast, no CPE and only small foci of infection were observed in T-ex5 PPMO treated cells at 72 hours post infection. (FIG. 5).

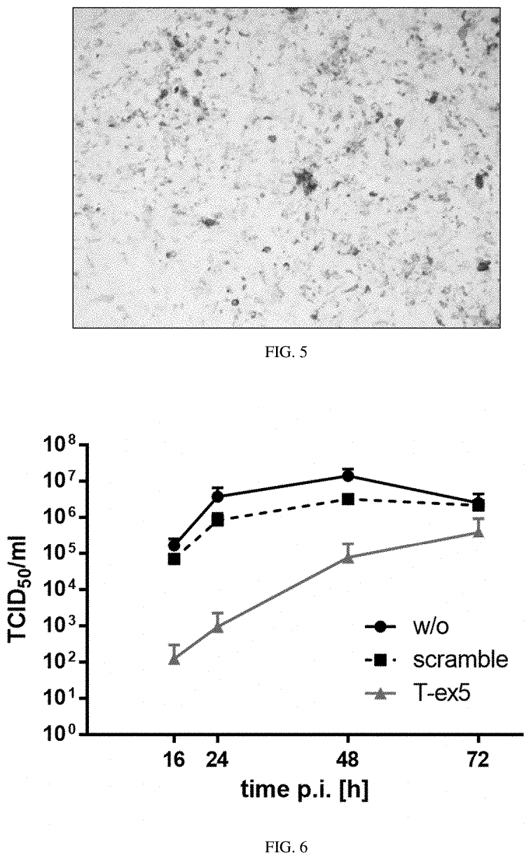

To examine SARS-CoV-2 activation and multicycle replication in PPMO treated cells in more detail, Calu-3 cells were treated with 25 μM T-ex5 PPMO for 24 hours prior to infection, then inoculated with virus at a MOI of 0.001 for 1.5 hours and incubated for 48 to 72 hours in the absence of further PPMO treatment as described above. Untreated Calu-3 cells and Calu-3 cells treated with PPMO scramble were used as controls. At different time points post infection, virus titers in the supernatants were determined by tissue culture infection dose 50% (TCID₅₀) end-point dilution. As shown in FIG. 6, T-ex5 PPMO treatment suppressed virus titers in Calu-3 cells by over 10,000 fold at 16 and 24 hours post infection, over 1000-fold at 48 hours post infection and around 200-fold at 72 hours post-infection.

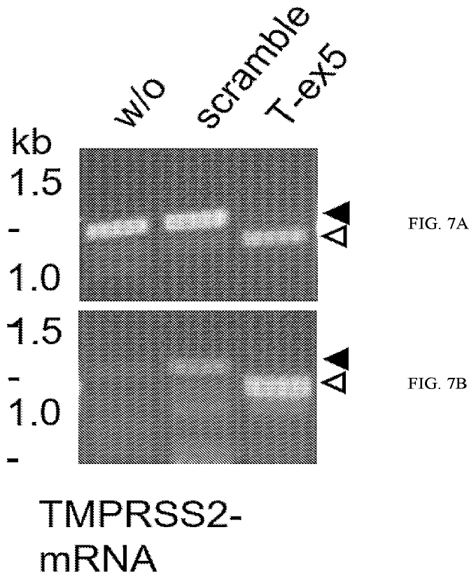

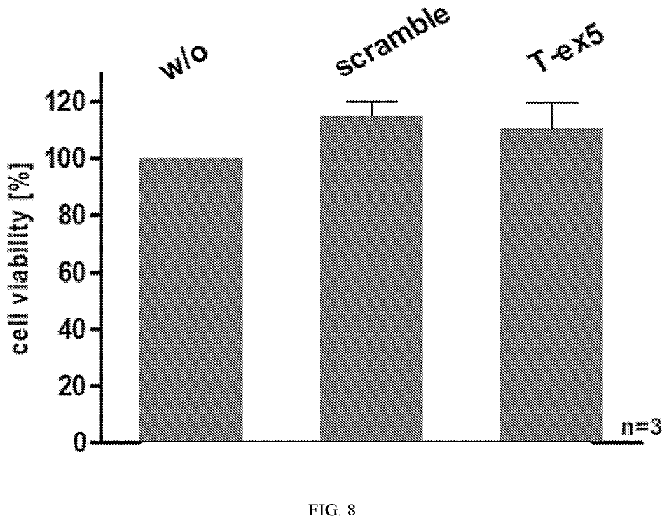

To confirm knockdown of enzymatically active TMPRSS2 expression, Calu-3 cells were treated with PPMO or remained untreated for 24 hours, and then TMPRSS2-specific mRNA was analysed by RT-PCR as described by Böttcher-Friebertshäuser E, et. al., (“Inhibition of influenza virus infection in human airway cell cultures by an antisense peptide-conjugated morpholino oligomer targeting the hemagglutinin-activating protease TMPRSS2” J. Virol. 2011, 85:1554-1562). Total RNA was isolated and analysed with primers designed to amplify nucleotides 108 to 1336 of TMPRSS2-mRNA. A full-length PCR product of 1228 bp was amplified from untreated and scramble PPMO treated Calu-3 cells, whereas a shorter PCR fragment of about 1100 bp was amplified from uninfected cells treated with 25 μM T-ex5 PPMO (FIG. 7A). Sequencing revealed that the truncated TMPRSS2-mRNA lacked the entire exon 5. To further confirm that T-ex5 PPMO single dose treatment prior to infection still interferes with TMPRSS2-mRNA splicing at 72 hours post infection, total RNA was isolated from infected cells at 72 hours post infection and amplified as described above. As shown in FIG. 7B, only the truncated PCR fragment lacking exon 5 was amplified from T-ex5 treated cells. The data demonstrate that T-ex5 was highly effective at producing exon skipping in TMPRSS2-pre-mRNA and, thus, at inhibiting expression of enzymatically active protease, during virus growth kinetics in Calu-3 cells. In contrast, the full-length PCR product was amplified from untreated and scramble PPMO treated cells at 72 hours post infection. The low amount of PCR product amplified from control cells is a result of the strong virus-induced CPE and thus analysis of lower amounts of cells by RT-PCR analysis compared to T-ex5 treated cells (cf. FIGS. 3-5). Cell viability was not affected by T-ex5 PPMO treatment of Calu-3 cells, as shown in FIG. 8.

Together, the data identified TMPRSS2 as host cell factor essential for SARS-CoV-2 activation and multiplication in Calu-3 cells and show that downregulation of TMPRSS2 activity dramatically blocks SARS-CoV-2 replication. The data demonstrate that TMPRSS2 cleaves the SARS-CoV-2 S protein and is important for virus multicycle replication in Calu-3 human airway cells. Hence, inhibition of TMPRSS2 can render the S protein of SARS-CoV-2 unable to efficiently mediate virus entry and fusion. Therefore, TMPRSS2 provides a promising drug target for treatment of Covid-19, and T-ex5 PPMO may be useful as a novel protease inhibitor for treating and/or preventing SARS-CoV-2 infection.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for treating or preventing a SARS-CoV-2 infection in a subject, comprising administering a compound comprising a steric blocking antisense oligomer.

2. The method of claim 1, wherein the antisense oligomer comprises a nucleic acid base sequence that is antisense to at least a portion of the transmembrane serine protease 2 (TMPRSS2) gene, TMPRSS2 pre-mRNA or TMPRSS2 mRNA.

3. The method of claim 2, wherein the nucleic acid base sequence comprises from 10 to 40 nucleic acid bases.

4. The method of claim 3, wherein the nucleic acid base sequence comprises from 20 to 30 nucleic acid bases.

5. The method of claim 1, wherein the oligomer further comprises a steric blocking backbone on which the nucleic acid bases are attached.

6. The method of claim 5, wherein the backbone comprises phosphorodiamidate morpholino (PMO), methylphosphonate, 2′-O-methyl RNA (2′-)Me), 2′-O-methyl phosphorothioate (2′-OMePS), 2′-O-methoxyethyl RNA (2′-MOE), 2′-O-methoxyethyl phosphorothioate (2′-MOE-PS), peptide nucleic acid (PNA), tricycle-DNA (tcDNA), locked nucleic acid (LNA), or a combination thereof.

7. The method of claim 5, wherein the backbone comprises a moiety having a structure 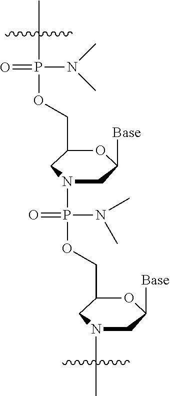
where each Base refers to the attachment point of a nucleic acid base.

8. The method of claim 1, wherein the oligomer comprises a nucleic acid base sequence selected from:
(SEQ ID NO: 2)
CAGAGTTGGAGCACTTGCTGCCCA;
(SEQ ID NO: 3)
TCCTGCTTAGCTCGCGCCTACTC;
(SEQ ID NO: 4)
TCAATATGACCTGCCGCGCTCCAGG;
(SEQ ID NO: 5)
AATGTTCAATATGACCTGCCGCGCT;
(SEQ ID NO: 6)
CCAGGTTCCCCTCCCCAGCCCGGAC;
(SEQ ID NO: 7)
ACCCTGAGTTCAAAGCCATCTTGCT;
(SEQ ID NO: 8)
GTGGTGACCCTGAGTTCAAAGCCAT;
(SEQ ID NO: 9)
GGTAATAATTAACCACTTACTGAGT;
(SEQ ID NO: 10)
GTGGTGACCCCTAAACAGTTGAAAA;
(SEQ ID NO: 11)
ACAAAGAGAATCCTACTTGAGGTGC;
(SEQ ID NO: 12)
TTCTTAGTCTCTGGAAGAAGGAGGA;
(SEQ ID NO: 13)
GATCGAGGCTCCCTGCACTTACTGA;
(SEQ ID NO: 14)
TTGCTGCCCACTTGCAGAGAAAACA;
(SEQ ID NO: 15)
GGTCAAGGCTGACTCACCACACCGA;
(SEQ ID NO: 16)
ACTGCACGAGAGGGAGGATTATCCA;
(SEQ ID NO: 17)
CGGGTGCTGCCCCATACTCACTTAT;
(SEQ ID NO: 18)
GAAGAAATTGCTGCATACCTGTGGT;
(SEQ ID NO: 19)
AGGCATCACTGCAAAAAGAACAGGG;
(SEQ ID NO: 20)
GGGACTCCAGATGAACTTACCTATA;
(SEQ ID NO: 21)
ACCCCGCAGGCTGAGGATGACAAAC;
(SEQ ID NO: 22)
CCGCCCCTGGCATACTTTTCCACGC;
(SEQ ID NO: 23)
CTGGGAGAGAAGAAGGACTCAGTAT;
(SEQ ID NO: 24)
CCAAGCCTGAGCCACACGTACCGTT;
(SEQ ID NO: 25)
TCACTAGGTCTGTTTCAAGAAGAGA;
(SEQ ID NO: 26)
TGCCCAGGAGCAGCCTCACCTTTCT;
(SEQ ID NO: 27)
CTAAGGACAGGGAGACTTGTTGAGC;
(SEQ ID NO: 28)
ACTGTCACCCTGTGGGACACAGCAA;
(SEQ ID NO: 29)
GGACAGGATAGTTACCCTCATTTGT;
or
(SEQ ID NO: 30)
AATAATTAGCCACTTACTGAGTTCA;
wherein the sequences are shown from 5′ to 3′.

9. The method of claim 8, wherein the nucleic acid base sequence is 5′-CAGAGTTGGAGCACTTGCTGCCCA-3′ (SEQ ID NO. 2).

10. The method of claim 1, wherein the compound further comprises a peptide.

11. The method of claim 10, wherein the peptide comprises from 2 to 60 amino acids.

12. The method of claim 10, wherein the peptide is selected from any one of SEQ ID NOS: 59-539.

13. The method of claim 10, wherein the peptide is RAhxRRAhxRRAhxRRAhxRAhxB where R=Arginine, Ahx=6-aminohexanoic acid, and B=beta-alanine (SEQ ID NO: 59).

14. The method of claim 10, wherein the peptide is attached at the 3′ end of the oligomer.

15. The method of claim 10, wherein the peptide is attached at the 5′ end of the oligomer.

16. The method of claim 10, wherein the peptide comprises one or more amino acids selected from glycine, valine, alanine, leucine, isoleucine, methionine, phenylalanine, tryptophan, tyrosine, serine, threonine, asparagine, glutamine, arginine, histidine, lysine, aspartic acid, glutamic acid, cysteine, proline, beta-alanine, selenocysteine, pyrrolysine, 7-aminoheptanoic acid, 6-amino hexanoic acid, 5-aminopentanoic acid, 4-aminobutanoic acid, or homoarginine.

17. The method of claim 1, wherein the subject is a human subject.

18. The method of claim 1, comprising administering the compound by inhalation.

19. The method of claim 1, comprising administering from 0.01 mg/kg to about 30 mg/kg of the compound.

20. A method for treating or preventing a SARS-CoV-2 infection in a human subject, comprising administering to the subject an effective amount of a compound having a structure 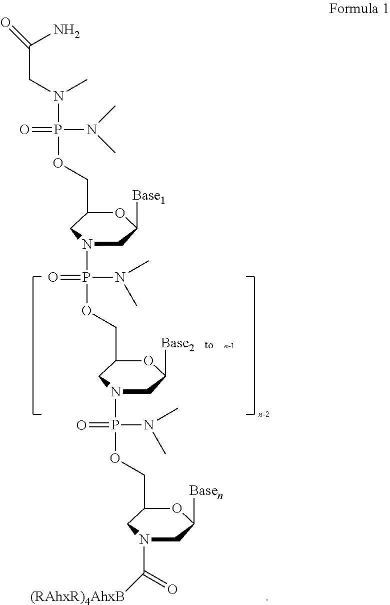
or a pharmaceutically acceptable salt thereof, wherein:
n is from 20 to 30;
each Base independently is selected from adenine, guanine, cytosine or thymine;
R is Arginine;
Ahx is 6-aminohexanoic acid; and
B is beta-alanine.

21. The method of claim 20, wherein each Base is selected such that the compound has a nucleic acid base sequence from Base₁ to Baseₙ selected from:
(SEQ ID NO: 2)
CAGAGTTGGAGCACTTGCTGCCCA;
(SEQ ID NO: 3)
TCCTGCTTAGCTCGCGCCTACTC;
(SEQ ID NO: 4)
TCAATATGACCTGCCGCGCTCCAGG;
(SEQ ID NO: 5)
AATGTTCAATATGACCTGCCGCGCT;
(SEQ ID NO: 6)
CCAGGTTCCCCTCCCCAGCCCGGAC;
(SEQ ID NO: 7)
ACCCTGAGTTCAAAGCCATCTTGCT;
(SEQ ID NO: 8)
GTGGTGACCCTGAGTTCAAAGCCAT;
(SEQ ID NO: 9)
GGTAATAATTAACCACTTACTGAGT;
(SEQ ID NO: 10)
GTGGTGACCCCTAAACAGTTGAAAA;
(SEQ ID NO: 11)
ACAAAGAGAATCCTACTTGAGGTGC;
(SEQ ID NO: 12)
TTCTTAGTCTCTGGAAGAAGGAGGA;
(SEQ ID NO: 13)
GATCGAGGCTCCCTGCACTTACTGA;
(SEQ ID NO: 14)
TTGCTGCCCACTTGCAGAGAAAACA;
(SEQ ID NO: 15)
GGTCAAGGCTGACTCACCACACCGA;
(SEQ ID NO: 16)
ACTGCACGAGAGGGAGGATTATCCA;
(SEQ ID NO: 17)
CGGGTGCTGCCCCATACTCACTTAT;
(SEQ ID NO: 18)
GAAGAAATTGCTGCATACCTGTGGT;
(SEQ ID NO: 19)
AGGCATCACTGCAAAAAGAACAGGG;
(SEQ ID NO: 20)
GGGACTCCAGATGAACTTACCTATA;
(SEQ ID NO: 21)
ACCCCGCAGGCTGAGGATGACAAAC;
(SEQ ID NO: 22)
CCGCCCCTGGCATACTTTTCCACGC;
(SEQ ID NO: 23)
CTGGGAGAGAAGAAGGACTCAGTAT;
(SEQ ID NO: 24)
CCAAGCCTGAGCCACACGTACCGTT;
(SEQ ID NO: 25)
TCACTAGGTCTGTTTCAAGAAGAGA;
(SEQ ID NO: 26)
TGCCCAGGAGCAGCCTCACCTTTCT;
(SEQ ID NO: 27)
CTAAGGACAGGGAGACTTGTTGAGC;
(SEQ ID NO: 28)
ACTGTCACCCTGTGGGACACAGCAA;
(SEQ ID NO: 29)
GGACAGGATAGTTACCCTCATTTGT;
or
(SEQ ID NO: 30)
AATAATTAGCCACTTACTGAGTTCA.


SEQUENCE LISTING (540 sequences)

SEQ ID NO: 1 - DNA, 73566 bp, Homo sapiens
  source 1..73566: mol_type = genomic DNA
  misc_feature 1..73566: note = Homo sapiens transmembrane serine protease 2 (TMPRSS2)
ccaccatgctgggttgtctgggtgcagatggatcgcccctagggttcatgcccaggcacaagtctgctggcacccactcagggtggcactgtcctggctggtgacttctccgcatccctacccatcttgactgcaccgtccctggctacacagtgtccagcctgactatactgaaggtagacacagccccgtccccctctcctctccaaatccagcccagctcccaaataccaggcaacattgaacaacaagctcagaccctgtgcttcctctggaaaacagggctactaacccctcctcccagtgttattccaaggatggaatgagagaatattcaggaagtatttatcaaaatactggacctatgacacatcacttaaaatcaaacctatttgattacagttctattatgaatgatgaaaattttaaattagaatatttattgtaattactttttacagtaggtatcacaggttctacatatcaaaaatattgaccataaactctcccttctatggctttgctcctgctgctctgggtgtcttttatcgattgccacaaaaccagagctgatgtggcaccatgaaatccactcacactcccagctcctcccgataaaatggtactcagctaataatgtctttcttaaggaagttacatgactctaggatttgtaaaaacagtagtgcatgtggttcttctgtcattttctctttattgaatttcaggacagggaaggcattttccatatacagatcaagctatggctgggtgggggtccagcttcatgtctccatcatcgatgccatgggtgaaattatgagtttaaaacatttatttatttatttatttttgagagagtctctgtcgcccaggctggagtgcgatggcatgatctcggctcactgcaacctctgcaacctgggctcaagtgattctcctgcctcagcctcctgagtagctggaattacaggcatactccaccacgtctggctaatttttgaatttttagtagagacagggttttaccatgttggccaggcttttcttgatcttctgacctcaagtgatccacccaccttggcctcccaaagtgctgggattataggcgtgagccaccgtgtctggccaatgagtttaaaatttagaaacagggctgggtgcagtggctcacacctgtaatcccagcactttgggaagctgaggcaggaggattgtttgagcccaggagttcaagaacagcctgaggaacatagcaagaccctgtctctacaaaaaataattttaaaaaacctagctgggtgtggtggtgcgtgcccatagtcccagctactcaggaggctgaggtgggaggatcacttaagcccaggagttcgaggctgcagtgagctatgatcgctccactgcatctagcctgggtggacagagcaagactctgtctctaaaaaaacacaaaggtttagggaaaaaaaacagacagaaatgctcaggatcgagccacaggcttctttcctaagtcctggcccttggcattttaattcctccagctggttccattctctcccaccccttggttttacatcaagctatcatcacgggtattctctaaaagtagaaacactttttctttgtctctttatcttggggttgacctggggtctgaaaaacacacgtgaaagaattttgaccattgtcagatgctgcccaagtatgacaaggagaggctgtgcttcctcagaatccagtgagcaccatgctggtgccttaggtacctcgtggctttcgtcttttcaatcaacctacagcatacacagtctttgtcccatctcatggatgaggtaactgaggcttagggaatttaagcaacttttctagactacacatctcaggaatgacggttctaagggttaagaccacttgtctgatgtcaaaatcaatgttgtaaccaacttgagcagaacacactggtcagccagtccctctttatagagcacataatcgtcccaagcacagagtgaattcctggaatcaagaggtgtcaagggggttatagtgtctggactccaggagcttttatccccaacaaaactgtctcccaagtaaagaactgtggcgttgattaccagaaatcctcctggtcactgactgcatttctccctgctctcctactaccttataccccagcagccagcacagtccctgctactgagctgggagcgccctgatggattctgctccccatcccttctccaagcatccttcaggtgctgtcctctgcccctcacccccaccatgcccagcagccccctccagctggccacagccacccccctatgccagccagccgcctggtcaggtgatgcctctctggctgcttctcattgctcccatgcagcatcggaaagggacatcccattggctttcactgacttgtgaaataaatgcaagagtgctcaggacttgtggatgtggacccgttgactctttgcccttggggtgggatctatattctctcccttttaagcttcatgctgagggcggtgcaggggcagtggctgagggagaagtggggggctttgtcagaaaattgcctacgacccgaaattcaagcttacaatcacagtgtcagtgtaacagccatgaagcaaaggacagggctggaatacatcgtttcttttcctgtgtttttttaaattattattattaaaagtaatacaagaaatccaggaagggccaagtgcagtggctcctgcctgtaatcccagcactgtgggagaccgagctgggtggatcacctgagttaggagttcgagaccagcctggtcaacatggcgaaaccccatctccactaaaaatacaaacattagccggacatggtggcacacacctgtaattccagctattcaggaggctgaggcaggagaattgcttgagcccgggagatgaaggctgtagcgagctgagatagtgccattacacggtagcctggccgacagagtgagactctgtctcaaaaacaaaaaaaacaaaaacaaaacaaaacagaaatccaggaagacagtaagctctgggtgcatgcactatgcaaaaataaaataaaataaaataaaccgtaatgtttagaagcagaggcagattccagggtctttcctaatgaaagaggtgatattttttccctgatggtagagaagatactgaggaaatgaccaagactttcttccccggtggcagcatggacaccctctttatttttcacctctggattaagagtggcagggcctagactccgggatgcctacagcttcctagcaacagcaggagagaaagacagatgtgtctgggggaagtcagtccccaggcgccccgggagtttgtccatggatgtgcacccctcagagaggttccttccaaatctaggtggggttctgagaagcccaggacccctccctgcatttcagcggactcagtagctgtcactggaatccctgtcttaccttaaagtcctgtttctaaaccttttcagccttggacatcgggcactgtgtccaggtctaagaagagaaggagccaatagccagtggtgtaaggttatttttacagccaatctaactcagatgacttaagaaaggaaatgtaccattaaattataaactcttcatctttggtgaactaaggactaaactctgattttttatcttgcccaaattcctatctaaggggtctgggaagtcatttcctacaaaccataaattttcgtcagatgggttttatttaaccctatatatcgtgactgactttccaatctgactctggcataacattatgtgacaaataagaaaatcaaagtattttaccctgaaacgtgtttcttggccatattttgaaatgccctgcaaagatgtcccttgtgggaaaaaatttgcgtctgtaaagaatctctattaacacagctagatcttgtttcttccaggccctcacaatcctgaaaagattaagagcctagcaccttttaaaggtctgaacaggaagcatttgtcattgattgtctctaagggcagccactgtgagactttggaagaaccctggtctccacagtcttttatcttaacctgagcatttcctttctatggatcccaggtctttagacaaacacaaccagttgtcaaccagaaaatgtttaatttggccaggcgcagtggctcacacctgtaatctcagcacttttggaggccaaggtgggcggatcacctgaggtcaggagtttgagaccagcctgaccaacatggagaaaccccatctctactaaaaacacaaaattagcccggcgtggtggtgcatgcctgtaatcccagctacatgggaggctgaggcaggagaatcgcttgaccctgggaggtggaggttgccgtgagctgagatcatgccattgcactccagcctgggcaacaagagcaaaactccgtctcaaaaaaaaaaaaagaagtgtaaattcacctatagcctggaagcacactccacccatcccccacccccccaattttgagttgtcctgcctttctagaccaaaccaatgtatttcttaaatgcatttgattgatgtctcatgcctccctaaaatgtataaaaccagactgcaccccaaccaccttggacatatgttctcaggacctcctgagggctgtgtcatgggccatggtcactcagatttggctcagaataaaattcttcaaatattttatagcgtttcactttttttgttgacacatggctgtgtttttatgtatttatcaacaatggaagcaaaagaagagggtacagggggctcacagaccatctcacagcgggtgaccttcaggcagaggcacctagggctgggtgtgtagggaggggtggcctgggcccaccccctaggtaaggctgtcatagacaacaggggatgccccctagaagtcacaatgcagagctgcttctcactcttccaggctgagatctgacttctcagcccaccagaggcaaaaagaggacagtctcacggtccccaagcccagagaccactgggaggggagcaagtggggtctggaccaccgaccttagcttcacctcaacccccaagaacacctgccccgttggaggtaaggcctggaaatttgcatttttttttaaaaagtctggctttctcaataactcttatatggaatgacatttgagaatcactggagatattacctatacacccccaacacacacacatacacacacacacacgcacacgcatacacacacacatgtgattgttctgaccagaaccttgagcattctgtcccttatatgaggtgcttctgtctctaatcacagcctccctggccaggagacccctgcttcccatctgggtcccctttccctcactgacagcaggcccgactagcactgatccccgtgatggcattttgtaaactcctgtggccagccaaccctgctcgttagtggcacattcttctgtttttgtttgtatgtttagttgtatgccgtacttttgattcatgaagataagatctcactggcattaagctatttttatcttttaaacatgaccttgaatacctatgctctgatcctgtgtgagctgcctttgcagttttattgtcagaaagtaattatgtcattactttcaacttttggggcaacagccttgtcagtcacttcatctagaacaacaaatactttattattattattaattctattattttgcttaatctcatgctataggcaagatcatggcctatgatcctgtctggtcctatgctgtgacattagtagtggttttgtatgtgcccagttatttttggatattaatttatgtatttttcttttgtttttcccttttctctccctcaggcagactggagctggctgtggattttatttctcaggagaaggaggtgggagctggagagagtgacccgtgcatctttccagcttttccttggccacctgggtggcatgggagctgggagtcagagttctgctgagctgtaggaccaaaggccactggctccgctgcacctgcggactgagacacccacctgctgagtgagacccagcctctgcgtgcttaggggtcccctgctacagggcattgaggtgaggtccgcctttgcccagtgtgagagcgctgctttcagagctttgagaaggctgttatcatttgtttctttttgaacttgccacctccccacttagagaatatttctggggacagagtttcgtaactcctgccgcatagttggtgcctgctctcgactacattaaatctattttcagtgcccctctggcaacaaaataatgcttttatttctcacatccctggcaacactgggtttatttttatttatcgttactgtctcaagtacgtgggattttaatactttaatatcatcctcattcctttccttccacgtctgtgccgtgtacatttttcaggtggacagaaattgttttcacagcttcttgctcattcatatctttcctggaatccccccattctgcctctcacaggcagccccagagctctgtccaccggctgaccctccctccctggggagctcctctgggccccggctccaagtagcacttccctgctggcgccccccaagttcacatctccatcccctcaactccaactgcctgccaccacctgctcttcccagtttaattagcacctcaagttgaacgtgtccaaggccaagttcttgatgtcatcttgcacatccgctttccccatctccgtcagtggcggctccattccttcagctgttcagactaaaatccctggggtcacctctgactgcccagttcccctcagtccccacattccatttgctagtcctccattcccactccccgctctaggatgtcttgagcactattgtacaagccttcttctaggatctcccctccttggcactgctcctggcacgtcgtttgtaacacagcagataggccgggtgcggtggctcacgcctgtaatcccagcactttggaaggccaaggtgggcggatcacttaaggtcaggtgttcaagaccagcctggccaacatggtgaaaccccatctctactaaaaaaaaaatgtaacacagcaaagagcagactgtttcttctgaagtgttaaccagggtatgtgatttctctggtcccagccatccagaatacaattctgaatccttcctgcagtgttcagggccttccataatgcactctcactgccctcatgaaggctcctcccccagcagaggcacctgctgcttctcctgcgtatggacaccctcccagactcctgccggagatggagccccttcgatggtcaggctggcctcttatcctggctttacatttttttttttttttggcactggcactgcacattcattatgtgttagtttgtgccttccctgctgggacacaagcttcaggaagctgcatgttctgctctctttgaacttcccagcacaggtgggcagtaaatgtgtgcagccatcctgcagagcagatgcagggggtgcaggtgtggggcctccatcgttttcctcattgtttcttttctttttctttttctttttttttgagacagagtctctctccatcacccaggctggagtgcaatggtatgatctcggctcactgcaacttctgcctcccgggttcaagtgattctcctgcctcagcctcccgagcagctgggattacaagcgcccaccaccacaaccagataatttttgtatttttagtagagatggggtttcaccatgttggccaggctggtctcgaactcctgatctcaggtgatccaccaacctcggcctcccaaagtgctgggattacaggcgtgagccactgcgcccagcgttttcctcattgtttctaagctagggaagttaggaagcggagagagtgcacagagggaagccaagatgtttcatgtgcttggcggcagcttctgacctccattctgtggggagacggggtctctggagctcgctgctgggcttcgggggttgaccggaccccagcgcctcctggaacagcttaggacagtggaatttcagttgcctgcagggctgccactcagatgcccgcagggctgaggagctccttcctgctcctctaaaaggtggcccagtgagccaggtcttccatgttttaaagagaagctggaaagctgaattttatgtgaagtctcctagttttaagatgtcagttcaaatgtttgtaaacttctgtgtttctagtaagacacatcgtacctggtttggcccactacaatcaactgacagctttgttctggatcaatctaaagttgattgatgcatttttaaaagctgatgcttttatttctgccctccagctgtatatgtaagtcaaagaacaattaagatacaaaactgtgattcagtgttgcaacccacgtagaagagagttcagccttaaggtttaatttctttcatcttacaaaatagggcatggacagataacctcaccatgagtttgttcaacgttttcttatgaatatgaatacatcatcctttccataagacattttaaggcatccgcaggaaaatgtcatgttaaatatacatatccttcatgactacaaatgtgaaagtcatccctacgtttgtaccgtatacaacctgaacaactacacatggcaaccctaggtatgggtgtctctgtccagcccagtccagccctaggctgctttgcaagttacctgaattatctacttcttggaaaatataccttgtatttgcccagctctttgtagtccacaagtctctctctattcattggttaatttaattcttcatgacccagcaatacgttattatgcctattttatggataaagcaactaattctcagggaagctgagcaatctgtgtatgagattacagaagtagttacaggtaccgtgaaaggcaaatctgtgctctgacctaaggggtccctggagctgatacgctccaccctgcatgatttggtgcatcttccttatcctttgggcaggtgggtatcctcacccctaccccagcacctacagtccaccctgggggtaagctccagtccctcccagtgtgctgcctggggtgaccaggaatcagtgttccagcgagcagttaaaggcctggctgagccagctcacaggcgctgagcacaggtgctttcgccttccacgtgcatctcacacacggggctgggtgacacccaggaggcgagcataatcactacacacatgcctgtgacctcctgtcgcctgtgacctcctgtcacctgtgacgacctcatcatctaaaagctctaactcaaacttgtgcaggaatcttgaaattcttgaccatttttttaggatctcagatattaaaacaattttcaaactgccattagcctaaggggttgcaggtgatttttgaaggacagagtaggtaaagaaggaagtaccttccccacgccccgctttctttttatagtagaattctttactattttacagcagacattttgttcccatgattggaagacttttacaaccaggaacatagccagttttattttctttgttagttttattgagtagtttatcactgtatatttacatatgacattggttattggcagatataggctctccagatccctaacacaccaacacaaaatgagcttattagtcacggtgataacggaagccacgcagtgcctgggaggcaggagggcaaggttggaaccccctgaagattggagttgggataaagtggaacattccctgtcgaggggtcgaggggtcgaggggttgatgaggagtgagtgtggaccatgttgcaccagcccaagcttggtggaaacagccaggcaaggggtttaagtgagagattcaaagaatcttagagcacaaagttgctgctttccattgaagagctgatgggtctttctggaggtttccatgagaaatttgccccattccatttactggggctggagtctcctggaacgactttgccagtcacgtgaatggaaacggtggaagagcaaagtctctccttaattacacagggagctgtttgcggtgtgaggcacaggtggtttaagctttcatttctggttttgttttttttttttttgagacagagtctcactctgtcacccaggctggagtgcagtggcgcaatctctgctcactgcaacctctgcctgcaaggtttaaggattctcctccctcagcctcccgagtagctgggattacaggtgtgtaccaccatgcttggctaatttttgtatttttagtagacagggtgtcaccatgttggccaggctggtctcaagctcctgacctcaagtgatcaacccactttggccccccataagtgttaggattacaggcctgagccactgcacctggccgaagttcttgtttcttaaaggacagattggctaaagcagctacagtttccaaaaaccaatgggaaaaatgggcacttgtgtatttgaatctttaactatatatttataaatataaatatatatttaactatatttatatttatgtatacatactgtatatatgcctgtttgtacacgtatgtgtatacacatgcatacacacatacacatatatgtgcatagataattaacttgttctcagcatgttacatatcccaaaatctggaagtagaggtgctcaagtaggatgaactatgttgctccttgatcagttggtttgcttcctgaccagatgttgggagtgataacagcaggctcacagtgtagtgcttctttgtccctgaagaaatgaaagaggagtgacctagccatctgcgacgggcagccaccccagggtcgctcctgttcagcacggaagcttgtcgcccccatccctatggatctgcgtgtttcacggaccctttgctcagtccctctttagctacacttcatatttgtttctctgcaaacacagagattcaagggttttatgagccttcctttgggccagccattttcaggaaaaaacaaaacaaacagaaaacaggtgcagttccctaagtggactctctgactgggcagcggggttttgggactgggaggagatggaaggggcaggggctgggtaagtgcagcagcagaagcagtgcctggccgtcaaggctttggtagccccagagtgacccagagcctcaaaggcgtttaagcgtgagcaccgtgggagcatccgagattgcccggttggcttaacttattattattatttttttttgagacggagtcttgctctgttccccaggctggagtgcagtggcacaatctcggctcactgcaagctccgcctcccaggttcaagccattctcctgcctcagcctcccgagtagctgggactacaggcgcccgccaccacaccgggctaatttttttgtatttttagtagagatggggtttcaccatgttagccaggatggtctcgatctcctgacctcgtgatcctcctgcctcggcctcccaaagtgctgggattacaggcgtgagccaccacgcccagccggcttaacctatttttaaaaagttacttgcatgtattttagctggaaaaaagaaatgtacaaggatcaaaaagcttattttatgattataaagacttgtgaacggccgggcgcaatggctcacgcctgtaatcccagcactttgggaggccgaggcggccggatcacaaggagattgagaccatcctggctaacacggtgaaaccccgtctatactaaaaatacaaaaaattagcctggcgtggtggcaggcgcctgtagtcccagctactcgggaggctgaggcaggagaatggcttgagcctgggaggcggagcttgcagtgagctgagatcgcgccactgcactccagcctgtgcaacagagcaagactccgtctcaaaaaaaaaaaaaaaaaaaaaaaaaaaaaaaaagacttgtgaacaaaaccttgaattttaggtcttcaaaaacttattctaagtggaataatgtttctcacttttctccttttcaaagcaatgaaaaaaaatatgatcctgattatcttcacccctgaagagactcctctttgttgggtgggagtggaggagggcaaagtcctagccttaggggagggcttcagtgaggtccattctatcagagccacctttctggaaataaaattgcatttggcaacagatatactttattttctcttgacagacagtaggtaatgtgttctgaaacacataataaaaactgtactactcatttagaaacaaattttagtaaaatgttaaaaatataggagaggtagattattttctatgcaaatgaagctttaaaactagtttcctgggcttccaaccacactgacgtcttatcccccagaacacaggcatgtgccccttcctactctggacctggctggggtgggcagaagatgggagggggcaggagaaatcagaggtgcacattcaacatccatacattgaggaaggaagtcactcccctcttgaacccttgcaggatgcccttgccctgaggtcaaactaagctctttggatgagtctgcgatgcctccagggtctcatctatccctgccttctcctcaaacagcagggactttcaaacctttgaaagcagcagcctccattttgcagatgaaatcttgcacaccctcttcaaaatgggccccagccgaggtggataacagagggtgttcagattgctggtgcctgccctcccttggcctccaccctggccatcccctccccactgctatgggactgccctgccccatgccacaggcagagccaggtaccagggctcctgtcctggggagggagaagcggctcctccaagctccctctgccccacactggagcccctggctgcctttcctcatcccttcctagtgcagggaactcccaggccgtggagaactcccaagggacgagatgatgacaaagatgctctgcttgatttaacccagcaatcccattactgggtataaacccaaaggaatatcagtcattctataacaaagatacatgcacgtgtacgttcattgcagcactattcacaatagcaaagacatggaatcaacccaaatgcccatcaatgacagactggataaagaaaatgtgacacatatacaccatggaatactatgcagccataaaaaggaacaagatcatgtcctttgcagggacatagatggagctggaagccattatccttagcaaactgatgcaggaagagaaaaccaaatactgcatgttctcacttataagtgggagctggacaatgagaacacatggacacagggagaggaacaacacacactggggcctgttgtgaggtgggatgtggggagggagagcatcaggataaatagctaatgcatgctgggcttaatacctaggtggtagattgacaggtgcggcaaaccaccatgacacatttacctatgtaacaaatcttcacatcctgcacaggtaccccagaacttaaaataaaaatgaaaattaaaaaaaaaagattctctgcttgatggaactttagtcaagctcctgagcttttcttagggcccatatgtgcacttccttctaaaatccagctccagcaaagaaccctgttaagtcagtttgatgagaactccaccctcgatatctgatggggttcctcaccctccaccatccctcggatgatgtttgatcacctcagcctgttctcagcaagggcagtttagccagaatccccctcacccctgatgtttccccttagtgattttctatccacggaccccactctgctcctgggctatcagtccccacttgcctgggctgtagttagagtggagcccaatctctcccaccccagaaagtcccattgccatggtgcctggacctattgcgatggtcctgagtagtccacctttccatctttaacaagggtctttgaataactttttcaaccatggaaagcaggtgctgcctttctgggaaggatttggggcactgatcttatcttgtaggccctttttaaaaaagccttgtgacacttcacccatctttgacatatacagcctttcattcaaaatggtgctttttcaaaaagcccaggatgtgagcttgaaatgtaattctggaagctgacctttaatgaagtttgtttaatcataaactcttattgttggagctagtgctgcatgtcttaaaagctttaaagggtacggcaggtactcatatctgattagcatctgcacttcaccaggtggccatttgttgtctaaaaatgttgacattcaacaatgtaatgaaaatgttggtcctggatgataaaaaaagtttttcacacagcaaggcagaggacagtgcactctgttgtggggcgtatgtctccctgcaccactaactagggcaggggtgaggaagtggtgggccacacctctgcctggaagacgtggtttttcccaggtacttcagaggctcttttcaatgtttgcaattgtagggggctcagaattcgccaggatttaacaggactggaggaggccaaattcactaggatttaataggactggaggagaccagaatgggctccccagaggcctcaaacccccattcctttctgtcacccagagtgacatataagcctcctccccacctttttcacgggggaagttcttattgcaatgatcctgtgtctccccatcattgtgaatggcctgtgttcggacctgttagagttatgacagaggccccagagccacctccagaagacctgcctacccagtgagaggacttctcagcagctggtgacccatcctagacacaggggattccccgtggagttgacagcagtgaggtgggaggagaaggaggcctacagacagggaagcaggagacgaagcagaccaggtgtctcaccctcagctgctgctgcactccccatgctcttccaaacacccttccccctgcaactgcctccctctgggttctggagctaagagagcctgcaggacttggggcccctggggacagaatgggagcagaacagccccacaaacagggttcccttcccaaatcctgaccccaggaaccccagctcctgattccaactcagctgcccatctcctaggggctgtgtggtgcagccaagtcaagtgccctctctgaaccccagcattctcagctgtaaaaaggggaaatcagtgtgtccatcttgcactgaggtaatgtaaactcgagtgtggtgactggcaaagcaaatattcagtaaaaggtaattgtttttatagcatacacagtgagccttggggctgagccttataacttggttcaaccatgaaaaacaacaaatattaaacaaccttttccttgtttgaacaactggcatctggaattcatctgaagaatttgctgaaagcttggtggttgtatgaagagagccatgaaagtctttgctatgatatgaatgtgtccccctaaaagcatgtgttggaaacgtaatccctgatggaacaatattgggagatggggcctaatgggaggtgtttaggtcatgaggcttaactcttaggaatggattaatgatgattagagggcttgagggtttgaggctatgtgttccagctcttgctgtctcgcctcgtgatgccttctgccatgttatgatgcagcaaggagaccctcgccagatgcagcccctcgtcttggacttcccagcctctagaaccatgagcctaataaacttctattgttaataagttacccagtctgtggtattctgtaacagcagcacgaaacagactaagacagtctttatgtttttctaaacaagggatctcattcatcccttgaggacatggccaaaggtcctcagcacaccagagatttgcttcacctggctcttaaaagtgctgatcattccatctatagcattctacacaccctgtctttacaaggaactcgtgaattctctgtcttgccttctgactacgagtacttgtgggaatcttagagctggttcacatcaaaactaaagtacagacactgggacaaacactgggacaaatattgttttattgctgacatcatgaatagctgcattattccttttcaggtacacacattttaaaatccattaatgatgaatcaaatgtcactttttttttttttgaggcagagtttcactcttgttgcccaggtgggagtgcaatgatgtgatctcggctcactgcaatctcccacctcctgggttcaagcgattctccggcctcagccttccaagtagctgggattacaggcacccatcaccatgcccagctaattttttgtatttttagtagaaacggggtttcactatgttggcctggctggtcttgaactcctgacctcaggcaatctaccggccttggcctctcaaagtgctgcgattacaggcgtgagccaccatgcctggtcaaatgtctgttaagaaacagattattgccaatactattttgcccagttccccagtttgtacccccacgcccattgaatctccattcattcccactaggggaaatcaccctactgaattttatgtttattaatttctttccctttttcacagatttagtatctaggcatatatttctaactatacataatttagctttggttgtttatgagctttatgcaaatgagatctgcaactggttttcttgcctagtactttgcttctgagagttatccgtgatggatacaatggtagttcaccatctttcattgttggagggcattgtgtcacatgcacacaccactgcacatttatctttcctcttgtcagtaggaactgacagaatttcctatcagtatttgttttctttttctccaggttttgcagtagaaatagtgcttctctgaacatgtgaaggtttttggtgtgttgcaagagtttctttagggtgcatacttagcagcgggatttccgggtgaaagattttcacatcttcaacattacagcatgatgctgaaaaggttttcaaagtggttgtatcaatttatatgctcatctcattccctctgcccccagcagatccccaattttggccatacctaatattgcgcaattttaatttttgccagtttggtgtgtataaaatggtaactcatttctaaaattttggtaaaatatacataacataaaatttatgattttaccatttttcagtgagtggcattaagtacattcacactgtcatgcaatcatctattttcaccgccatctattttcagaatgttctcatcttcccaaactgaaattctctacccattaaacactaactcccaacccttcaccttcgccccttagcccctggcacctgccattctactttgcgtctttatgactactttggacttctctagggacctcatatcagtggaatcatacagtacttgtctttttgtaactggcatatttcactgagcatagtgtcctcaaggttcctccatgctgagcactcatttggttttgtgttccacttgctataatgatgtggtgtgtgcgtgtttgtgtgtgtgtgtgtgtgtgtgtgtgtgtgtttaaaagggattgcttagaggatttggccttttgggtcaaactgtgggatctgactaagaggtctctcaatggctcttatctttgcatctgatggcatttgggaagggaaggaggttataaaatgaggaagaacaaggacaagctggaaccaatgagcatgagctggagcccacaaggccagaccattgcctataacagtgacaagagggtcaagttggggctggggcatgggctcatcatcttgaacatacacacctggccctggaatcagacgcgctgatggaagatccaggggaaggcagagcagttgcagatttaggcattcttctttgctttattttgcttggtttcattaagtcatctccttctcgctcctctcatcccattttttaattatttgggtatggatgctaatcatattatatatatatatatgtgtgtgtgtgtatatatatatatatatgtatgtatatatatatatagtcttttttccaccagaagaatacaatgattaaaaggctttaattgtgttcactttaattgtgttcatcgcactactggctttcatggtatggttttccaatacttcagttccatctttgtgttgtttttgttgttgctcttttacaagttaaatattactactggagtttctctatgctgtaaatattagcttagatttacccacattgtcaggattttattggttcaccattttctcttgcatgtcagactttctcctgggattattttcctgcttcctgagcaatatcctttagaagtttttttgttgtggtcaacactgttttgttttgcctataaatgtattttccctctctcaaaggatattttgcaggatataaaattctatgtagcctggtgtggtggctcacgcctgtaatccaaacatttgggaggctgaggtgggcagatcatggaggtcaggagttcgagaccagcctggccaatatggtgaaaccctgtctccactaaaaatacaaaaataagccgggcgtggtggcaggcacctgtaatctcagctactaagcaggctgaggcaggagaatgccttgaaccgaggaggcagaggctgcagtgagctgagatcaagccactgcacttgagcctgggtgacagagcgagactctgtctcaaacaaccaaacaaaattctatgttgacagccacttttcctgaagactttaaatattttattccactatattctagcttcaatctggatgttgtcttttgttttataatgctttttatttctatttatttatttattttaagatggagtatcgccgtgttgcccaggatggagtgcagtggcatgatctcagctcattgtagcctccgcctcctgggttcaagtgattcttgtgcctcagcctcctgagtagctgggattacaggtgttcaccatcacgccaggcccataatgctttttaggtcttctcttcacttcagggattctgaaatttcactaggatgtgtttctacaggttcctctttttttctgcttgggatttgttgtgcttcaataatctcagactttctgacttttatcaattttggaaaattcctgactatttattattttaaatattgattctgccctattatctcaatgatcactttccaaaaaactaatcagacacattttagatttttttactctattttctataccacttagtttttctcatattaaaaaaaaaaacaactttgtcttcctaggtgaattttgaataatttcttctggtctatattccatgtcactgattccatcttcagctatcgaaaactttcagctttttacttctagaagtcctctttagtttttttctctgcaattctgccaggtaattttaaacaatcctgattcccctgaggacccatcctaccaccctttgctctagacctgtaactaaactcaagttataacaggctcctaaaggtgaggtccaaagtgtgacccataaagaaatgtgctgcattccaaaagaactacttgagttttctaatttatacaaacaggaatccagggaacatgtgtggaaatggatattaaaggtatggataatggtggaaggaacacaactttggatcaaaccaaattcattgaaatgggcttgttaagcaaggactctgcagtgaatgttgcagctcagggagttagagagggctctaacagtttggtcggttggctgaaacatggatcaaaaggtgacccacagccagcgaactggaaatgctggacttgccttggtttaacatagagaaaaggattcaaatacttaggaagattgaaatgtgagggtggatttgttatttaacacctgcttacccactgtgggaggggccagtgtatcttacttttcaccagtactttgataaataagtttgtaagaggagcctcagcatcgtaaagagctctgtgattactctttggtctaggccagactttacgtgagaaatgcaatcactaaattgggaaacctcagtgcaataagagtagatggatcccaaggtagcaggggccaagtggaagcactcaactgccaaaggcaaggtgggcatggttactctaatggacagtgggtataaaacaaaaatcagaatagtccgactcatgtggatctatggcatgaactattcatggtgtcttgagacatgaactagatgggaagcctactaaacccttacttgatttgtatgagcagacaagttctagggcaagtgagcaggcctaacctgaatcatcaaaacggagactcatgcccctcaatcaattcccaggcttgagccagtttacaacccagaaccctttgagtgaagggaagactgggtccccttgaggaaggatcccaggacactattgaaaatggatcctgttagcctttctcccatcctttcccaaaggaaagggacctctggccttttaccagagtgactgtgcattgtgggaaaggtaagaatcagaccttttggagatgactggacactgcgtctaaactggcattgattccaggagactaaaaatgtctgtgtggcccttcaatcagagtaggggcttatggaggacaggtgatgaaaggagttttagcccatgtccagcttacagtgggtccattggttctcaaacccatcctatggttatttccttgttccagatgcataattggaatagatatccacagcagctggcagaatccccatttgggtcctcgactcatggaatgagggccatcatagctggaaacaccaagtggaagtcaccagaactgcctctacctaggaaaatagtaaatctaaaacaatattgcatccctgcagggattgcagagattagtgccaccatcaagaacctgaaagacacaagtgtggtgattcccaccacatccccattcaaccctcctgtttggcctgtgcagaagacagatggatcttggaaaatgacagtggattattgtaggcttaaccagctggtgactccaatggcagctgctgtatcagtttgagccaatgaacacatctactggtacctggcatgcagctgttcacctggcaaacaccattttcctgtccatgagacccaccagaagcagttggctttcagctggcaaggctggcaatacgccttcgctgtcctacctcaagggtctatcatctctccagtcctatgccatgattttgtttgcagggaacttcattgcatttcttttccacaaggtattgaatggtgtattaccctgatgacattatggtgattggccctagtgagtgagaagtagcaactactctagatttattggtaagatgtttgcatgtcagagggtgggaaataaatctgaaaaatttcaggggccttctaccttagtgaaatttctaggggtccagtggtgtggggcctattgagttgccttttctaaggtgaaggataagttgctgcatccggcccctcctacaaccaaaacagaggcacaatgcttagagggcgtctttggattttggaggcaacacagttggatgtgttactctgggccacttactgagtgacctgaaaagatgctagttttgagtgggccccagaacaggaaaaggctttacaacacgccaggctgctgtgcaagctgctctgacacctgggccataggacccagcagatccaatggtgcctgaagtttcagtgacagagagagatgtgatatggagctgtttgcaggccctggaggtgaattgtagcacaggccctgaggatttgaagcaaagccttgccatcctccacagataaatactttccttttgagaaatagctcttagccagctaccaggccttagtagagatggaagacttgaccatgggccaacaagttaccatgtgacctgagctcccgtcgtggactggatgttacctgccccaccacgtcataaagctgaggatgcacagcagcactccatcgtcaaatggaagtgtctatatgtggtcaggcctgatcaggtcctgaaagcttaagtagattacataaagaagtgacccaatgttcatggtctccactcctgctacactgccttctctgcccccctgcctgcacccatggccacatgggaagttccctatgatcaggtgacagagaaagagaagacccaggcctggtttacagacggttcagcaccatgtgctggcaccacccagaagtacccagctgcagcaccacagcccttttctgggacaccccagtaggattgtggtgaagggaaatcttcccagtgggaagaacgtagggcagtgcacctgcttgtgcactttgcttcgaaggagaaatggccagacatgcaattatatatggattcatgggctgtaatcaatgttgtggctggatagtcagggacttggaagaaacatgataggaaaattggtggtaaatttgggacagaggtatgtagacagacttctctgagtgggcaaaggatgtgaggatatttctgtcccatgtgaatggccaccaaaggatgacctcagcaggagaggactttttttttttttttgagatggagtcttgctctatcgcccaggctggagtgcaatggcatgatctcagctcactgcaacctccacctcgcaggttcaagtgattcttccggctcagcctcccgagtaactgggattataggcacgtgccaccatgcctggctaatttttgtatttttagtagagacggggtttcaccatgtcagtcaggctggtcttgaactcctgaccttgtgatccaccgaccttggactcccaaagtgctgggattacaggtgtgagccactgcacccggccaggagaagactttagtcataaagtggggccggggtggattgcctgagctcagaagttcgagaccagccttggcaacacagtgaaaccccgtctctactataatacaaaaaattagctgagcatggcagcaggtgcccgtaatcccagctactcaggaggctgagacaggagaatcacttgaacctgggaggcggaggttgcagtgagccgagattgtgccactgcactccagcctgggtgacagagtgagactccgtctccaaaaacaaacaaaaaaatcaagtggatgagatgacccattctgtagataccagtcagcctttccccagccactcctgtcatcacccaatgggctcatggacaaagtggcctcggtggcagggatggagattatgcatgggctcagcaacatggactcctactgaacaaggccaaactggctacagccacaactgagtgcccatctgccagcagcagagaccagcaccaaggctctgatatggcacggttccccagatgatcaggcagcaacctggtgtcaggtggattacattggaccaccttcagtatggaaggggcagcattttgtccttactggaataggcagtctggatacagatttgtcttccctgcacacagcgcttctggtggaactcacagaatgcctcatccaccatcatggtattccacatagcattgcttttgaccaaggaactgacttctcaaagaagcatggcaatgggcttatgctcatggaatcgactgatcttcccatattccccacccccatgaagcagctgacttgatagaatagtggaatcgccttttgaagatgcagttacagtgctggctaggggacaatgctctgcaggctggggcaaggttctccagaaagctgtgtatgctccaaatcagcatccaatatgcggtgctgtttctcccatagccaggattcactggtccaggaatcaaggggtggaaatgggagtggcaccacgcactattactccaagtggcccactagcaaaattttcgcttgctgtttctgtgactttacgctctgctgacctagaggtcttagttccggagggaggaatgctgccaccaggagacacaacaatgattcaattaaactagaatttacgactgccacctggccacgctgagctccacatgcctctgaatcaaaaggcaaagagagagttatgcattggctggggagacccatctggactaccaaggagaagctatagactacttctactccaccaggaaggtaaggaagagtgtgtctggaatacaacgattccttagggcatcttttagttttaccatgcttgtgattaaggtcaatggaaaactacaacacaatccaggcagggctattaatggcccagacccttgaggaatgaaggtttgtatcactccatcaggttaagaaccatgaccagctgtcatgcttcccgaaggcagagggaacgcagagtgggtagtggaagaagacagttacaaacaccagctattgcccatgcaacctgttataaagcattagattgtcacgagtattttctcattatttttttctaaatacatttgtgcacatatttgtatatacagtatatatctctgttgtcttttctctcatattcctttattacataacttaaggcatattgactttatatcattgtattaagtattgttaattttacattatggtatttaagttatggaataccaaggagaagtgtaaacactggagaattttacatccacttctggggaagggcttagtgcattttttggttgtatgcagaagagttttatcacattaggcaaaattattactttgctattgtctttagagataaagtatggtgtaaggagatgtgtaggtgtgaagtcgacaaggggtcaacttatggtggcatatatattcatgcttagataaaactaaggtcacattctgtcatacaagctggagtacagtggcacaatcaaagctcactgcagcctcaaactcctgggctcaactgatcctctcactttagcctcccaagtaccttgggctacgggtgcgcaccaccacacttggctaatttttaataaactttttgtagaggcaggatcttgctatggtgtccaggctagtctcaaactcctgggctcaagagatcctcccacttcagccttctaaatggtgggattacaggtgggagccactgcatccagccatggttaattttatgtgtcaagctgacttggtcatgggatgcccaggtaactggttaaacattatttctgggtgtctgcgtgtgagggtgtttttggaagagattaatgtttgaaaagactgtgtaatgctgattgccttcaatttgggcaatcggttgagagcctgaatagaacagaaaaggggaggaaggttgaatttgctctctgcctgactgtgtgagctggactttgatcctctcctgccctcagcactcctggttctcaggccttcagacctggactggaatctgttccatctgctctctagttctctggctctcaggactttgaaagctacaacaccagctttcctgggtctccagtctgtagatggcagattgtagagcttctcaccctccataattgtatgagtcaatttcttatagtaaatctttatatatattataaataatatttattacatataagctgtgtataatatatatcatatctgtattatattatgcatataaaatacatgtttatgtatatatttatatgtatttataatatataagatatttataatatttataaataatatatagaaatatagatgtgtgtatatacatgtgtgtgcatatgtatatgtgtgtatatatttgtatttctcctattgtttctgtttctctggagaaccctgattattaatataaaaggaaaaataaatggtgaatgaacaaccactccaagaggcctgagtctgcggcagcacttactccctggtttctgggtggaattagtaggcaccaaacacactgtcaagacagaaataagtgagttgccgctgaaagacaactgcacctgccctatgtgctggagagggtctttgagggtttcccactggggcagagcgcagggcagtgtgggggatggggagggctggggcatattggggacaatccctgttgtgggtgccaccagcagaccacaaaaggtgccaaaggagggagtgagagggaagggtttactaggaaagtcctgccccacaggaggcttccgggagagcttcccaggaagggagcagtgagccaaggcagcctgggatgggactgaatggggcttttttctgcttccacctcattttaaagcaaatcatgttgatttgtatattatgcaggggaggagaaaacatgcttcccctctacccttctagttcgttcagctagtctacaaattaaattggcatcaaatagatgaacaggagaaaagctgttttaatgtatgtactcacagatgggaatcccacaagaatatgagacttaaagaacaggccaggtgagtgaggggtccagtgcgagggctcacacctgtaatcccagcactttgggaggccaaggcaggtggatcgcttgagcccagattggagaccagtctgggcaacatagggagacccccatctctacaaaaaattaaattagctgggcgtggtggcacacgcctctagtcccagctactcgggaggctgaggtgggaggattacttgtgccctgggaggttgaagctgccatgagccatgattgagccactgcactccaacctgggcaacagagtgagaccccttctgggggaaaaaaaaacacacgaaaaaaaaaaggtgcagcagcccgatgattgaggcttatctgtcactctgagtgacagaaagaaatgggggtttgaggcttctggggagcggtggaggagtgaggggagcgtgaggagaggaggtgtctggtgaacgcaggttgccgtgtgaggcagataaaagtttcccaggtgataaaagttgtccgggaacagctctcttcctggtacagatctgctgactaacaaacatttcctttataggtgcaaatttcctttacaaaagggcattttctcagaggtactctggtgtctgcagttcctcaacataaccagttccaaatcatcaatgtgccaaagaggactatgttggggtagcagattctggtctcctccagtcctacttggggtgatgaattctggtctacggtcctattaaattctggtgaattctgagcccccacaattgcaaacattagaaagaacctctcaagtgcccgggaacagccacgtcttcctggctgaggtgtgtcccaccacttcctcactcccgccctggccggtggtgccgagagacctgggaccatccgggggagcactttccaccggacgctggtgggggccaagaaatgccagcctaggcggactggggagggtcttgggcgtccggcgctgtgtccccgccactcgtgcttgggccagcagtccccaaggcctaccctgggtccttgcccagaggctacagtgggttccccggaggccaagacggggccggccgcctacaggagctcgtgaggtagcagctccgggggctcacccaggactccaggagcgctccccagaatccccttccttaacccaaactcgagccctcgggcagcgctgcgccgcggaccggagaggggcaggttggccgctgtggccgggcccgggaagcgccccagagtcccttatgggtccctccgcagccggggttgagccaggcagggaacccgtcccggacttcccttgggaaacgcctcctcccgccgcccccgcccccgcccgcccagggtgacccgcgacccgcttgggggtgtcgccctggaccctgggacaccgcctcctgagattaaagcgagagccagggcgggccgggccgagtaggcgcgagctaagcaggaggcggaggcggaggcggagggcgaggggcggggagcgccgcctggagcgcggcaggtgagcggcgccggtaccagggtcccggctcggggtccgggctggggaggggaacctgggcgcctgggacccgccgatgccccctgccccgcccggaggtgaaagcgggtgtgaggagcgcggcgcggcaggtgagtgcgcccgagggtcgagcgctggggccagccgggcagggcgcctcccggggtgctgggagagtgctgggcgcctgggaccccttgcgcacggccgagagccctcgccgcccctgttggcttggcatcgcgaggttggggcaggtggtcctgcgagtccctagccagttggtggaagagagtcccccggggtccccaaagctggctcctagtccgcctgccctccacggcccccgcctgggagcaccggtgcgccttttctctttggggaggaggactgggagtgctgtcggttggcttctttgtttgggggaaatctctgcagtcccgcagtgttacctgaactccttctgcttcttggttccctactgaggtagggagaaactggaaactcgctgagatgccgggaacgatacggccggcgcggcggggaccgctgggccaggctgctggagggcccccgggcgaggtgcgaacggccagtcagtctcggggagggactgctcgcgactcaggccccgttgcggcgacccgctggcggcgatcagggccggggcctcctggacacccggcaccggcaggaacccgcccgggaggagccggtctggctcctccgcttgcgctccgtgccgggttggggggaaggcggggcacctgggcgcttcggtccacctcatcctgtcctgcccggctcggcacagtggcaccctgagtggtcaagtgctccagtctggcagtgggcgctctgctcgagcacgggtccagctgaaaaactgccccatgtccagaaagtttgtgcgccgagagccctgagcgcttgtggcagaggagggccttcgcccgctccgccgtagagcacagcgcggtggccctgaatcctgttcagaacgccctggtggcagccctaatcacgaggaatcctggtggcttgtttgggaaggaaaggatagcacggcggcccgaggtgagccctgtgggcagggagcacccagggctcgccttcgaggctgcgcagcccttccctccgtgcgaacctgtgcagtggtcccgcgggaaagatgagcgcatcgcgcccaaggcaatgcctgacctcacggagctcccagccgcgagaacctcccgaatcatggaaaattacaaaggctacgcagcccagttacaaggcatttggaaaacctaaagtgagactaagtttgttgacctgtcccaggagatctgggaaggtactccaggaagtggggatgcagccagcctcccctgaaggaaggatgggggttgggagtgtggcagggagagtttaccagcgcaaagggccttggggacgcaagtagctaagtcagtcctggctcctgagctgctgctgtgaagggaggacctggtgggctaggtaaccgggagctcccccagaaagtcactggaggttgctcaggacttccccacctttactgagtgcttttgaagaagtaggacctgccctgctccgctacagaactactggacaggtgaggttagaatgaaacctagcaacaccctcgggtaaggacacactaggctggagagttcggggtgcaacctacgggctgagacctctgcctggagatggccatctacacctcggcctctggttatttttaattatcagctggtgagctggctgggcttttgttctctgcttggacatttgtttggtccccagcagcttcccttattcccaccagccaaggacaaccttctaacaacaagcttgtgctgttgctggagcctcaggcagggcctcccaggttgctaggtgctggcctgcctggcaccgttggccactctatcttgaactcaagtcaccttaattgtaggagtatatttggctgcactggcattataataataaccacagacattgatgtggcacttttttcagccaggcatcattgcaaaccctttataatgtacttaactcaattgtaaggaaactggggaagagaggttgggaagcttgtccaagatcttgtaacctgtaaggggcagaactggggataaacatggcctgtctggctccataggtcagtgcttccagtgtcgggctctgagttggagacacaggatgcttagaaggggctttagtgacaatcggcacagtacggattccttttgatccttccctgggttctccctccaggatgttggaatgttaccagcaagtggaggaaaggagggaaggaagctggaaacaaggcgcccggaatcctgattgaaaagggcttgttcctgggccaggctctggttctccccgacctgaaagagcccagcccctgcccacagccctacctgggctccgtgctgctccctctagcagtatgacatggccagagtgcctggaactctgaaagggggcctgcggagggctgacctcctaggccatgggggcattgagtgacaagacgctggtgcaaatgtcctgtaggtgaaaaggtagatgcgtgcagtaccttatgccagtaaccaccaccaccacgatcatcatcatccgagtgtgttgtccccctaccaggaaatgtaaaccaaaagtctggctgataactcttacccttttccacctgaatctgcggcaagattccctaggggatcttcgtaataatatgggatcattgccttggtcccaattattacatcagactctgagaatggggcccaggcatcttatttatttatttatttttctaagccctcttgaaaattctgggtttggggagcactgatcagatctaagccttggacattgaggtagaaaggaaaggtgaatggtcaatggagtgcttgttcagttttggttgctgaagacttggaaagtggccttgctagtggggagagtgagggcagggagagctccctgtggggaaccagagagcccaggagcattgaaggcccaggtgcatttctgctctctggtccctcctgctctgcactccctgagtgtccctcatcccacagtcaccaggcctttgctactcttccccgcccctccttactttccaggacacctgccaggggatcctccaatatgggatgcactgcgtggcctcaggattggggtgcaggagcattatgtacctgagagctgaggcctgtatggagtcagcctttgtggggcccctcctggccctaagggtgatttgggaagccctgacttgcaagcaccaggggtatgggcctgggtgtgaagaagggggcttatgatgtccccatggccattgaccaccttggccccaccgagcctgtggacacccaaggaggagcaaaagggggatctgtgacagggtagcagcttggttgagcacacaggcagctcttcagcatgacctagaacagaggctgggctccaggaggaggcagagcctgatctgggacttggttattcctggttgctgagcaatggtcaggagagaggaactgaatgagtggaaatgcgctggttgggagtcgaggctggttttctggcaggggtgtgggtggttacttttgttgtaaacagagtgaagtttgagagggcaggaaactcgaagacaggcttttcatcctcctggaggtgagtctgggagagggaggtgagggctctgacatctatgtgtggatggcgctgagccattgggcatccatggcaggagggcaggaggagtttagggcacagagttgtggtaagaaagcaggaagtgtttgaagggcaccttggcttgtggttgtggcagcagaagtctggaagttcactcaagggcagactaggtggaaaaatcggggctgtcaagtgggactcagaatgagaggttgtgacaattggatttgttagaaaaatattttcaagcgaggtctgacttggcagatggaactaagctgaggctttttattattgcttgtttgtacgaggtgcacaatgcagtgtcaaattccctttaaggccattgctttccaaggggctgtcctggcggggcactcagcctgaccccactgcagtatagcccaggggtcatgggagaccatgctttctagggacgatgggcttggagttctttgtgcaagatactgataacagagaatttgctgacattttcaagttcactggtgatgatgcatttataaggagttgggagtaatatgaaagatcactatgtctggtcttgatgctcaagcctaattctgttctggaatatgttggagaaatgatcctccagtcttgaaacatacatttttaggtttgaggaattcggtacattctaaggtagctccaggccatatgtgaactttctaattgttggaaccttgatttccccagatagaatccaagcctgctcttctggaaattctgatcatgattctggtattgtcctttggatgtatgcagaataaactgaattccttttccacatagccggctcttacgtgttggaagataaaccttatctgccatcattatgttattattgagtgctcaccatgggttgggcactgagctacggccttgctttctcacttaatccctgcaacaaccctatttctgtatcatccccattttacagattgacttggacaagttctttaagcctcagccagtggcttgtgtgtggtgttgaagtctccgcctggagtgacctgagggcctctgccgcctgcctgcctgtcatgccaggttgcactcagcttgaggcacctggactcctgggaggtcacccactgtcctgacttgggctgctggtgttgtctgggtgtgggcatcttatcgccctctgctaaatttcatcttactcatttggcctttggaactcaaaaaatatatattttttcctgcaaattgcattgtatttcaaatttacatttccgatcgtctgttgttggtatataaaaacacatagatttttatattgatcttgtatccctcaactttgccaaacatgcttattaatttttccccaatagtttttggggtacaggtggtaagaactccataaaaacatttaaatactgtctttgtaagctacaaagtgagatttaaaaaacaaacagaaagagagagaaccagacctgatatctgccctaaaaagtttgtaatctctttgggacaatgagtcattcacaggaaaagaaacttggatgatgcaggcaatacctctcctaaggtgatctttctgtttctcgctcaacctttctctctcagtagaactcttgtgccaatcattgtgtatgcaatttacctgattatacataaacaagtttctgctgaacagccactgaaatccactgttggctaactcatttagtcatgcattctatccttaggaggatagaacatctcgttgtataatgtgatattagtgcggttattttggcataaagggctctttttgatgatttggtcattggctgggatgtgtccgtggatggtggtggtggtgctgtctggattttgaagaaggggcactgagtctgaggctgtctgttccctctcacacctccagcaactcgaaccgtaccagtcacaggtgggggcttcgagaaggagaaggtgtgtgcacaatggtgcacggtgaggtcagtggagacttcattgcagagaatacccctggatcttgggtgggagtcagtgtctacaagcctaacttcattcgcgttggcttggttgtttggaaagtaagtctccagtaaacgtgtcctgagatctccagaggagagaaactaggagaaaagattcttgttttggcagtaacttcatcatgtcatgggatgcggatacgatgccacagcatcgtcctgctgcatggtggccaggcaggtgactcatgggctgactcagctctcagtccaggatggaccaaaacagtgcgaggacagctgtgtgccattgtgagattcaagtgggtgacactgtctgtattgaaggaagagctccctgccagccaggaatggagcctgagtgccacccactgttgctctttcacacttaaaggaaaatttccccaagtgctcatggtggctggctttccagaattgagagccatcatcccggtcctttaaatgacaggagcaagcattcgctaaacagcattgtctctcgccatctactttacgtcttgctcctttcagaaatgagttaatgcgatgtacaagcttgtgtgaatgaggaaatgcctgttttgaagatgaacaggactgaatgtgatttcctggtcttttcgggacagcagtgcttccccttgcagatctaatgtcagagcgggacattcccccccttgttttattttcaattggagaatgttgttgttgtttcttttttttttttttttttttttttttttgagacagagtctctctctgtcacccaggctggagtgcagtggcctaatcttggctgactgcaacctctgcctcccaggttcaagtgattctcctgcctcagcctcccaagtagctgggattacaggcacccaccatcatacccagctaatttttgtatttttagtagagatggggtttcactatgttggccaggctggtcttgaactcctgacctcaggtgatctgcccgccttggcctcccaaagtgctggaattataggcaggagccgccgtgcccggcccagctggagaatgttactggcagcatcactctggtcttccattccaaagtccatggtaactgacttgggcacacggaatatttttgtttatcatgttctgattgctggaagagataggattatcctgtgactgcctctgggtcccatggcttttatttgacaattaatggtggtggctcccggtgttccttccttccctggcttccttgttagggatcgtgaatgaatagcaagtgtgggttttgagtattgattcttcactcctttcagagggcttctgagctgggagagctgccaatgggatcgaattttcacttcctgaaacttcaaccaaacctgtaactggggagataggacagccgagaaaccagaggcatgaggatgtgtttggttaggcaggactctttgaaagcaatcttaggatggtactgagttctgtcctgcttatgagacaagaatgcgggatttattgtgttgtcaagcctgaggatcctctctcttcacaaagcagacataggcaaggtctgttaagatgtggctaaaaggcttaaacgtttttgatacattttaaatggtgatccttcccacctgtacattagtcactttcctttccttatacccaggaaggtgtgaactttagttttaatcctagaaaaagtcccaaagctacaacatgacctggttcttgtatttttatctgtaattgaagagtaaataaatgattggtgcatgtttgttgtaacctgttgggtgaggtttctcgagactaaaaagccatatactcattttcattcatggcaggctgttggggttttatagtttaatgattcagcaaccagttctgcttaggacacaggaaagcgatggtgtactgaagccggccccactctacttggctccgtggtgggcttcatgttggtagcctgggaatacttaccgaaggcaaacctgcgaatcaagacagccctcttttccttcagcaggttgttaaatgtttatcagctttataattcctcttatttaatggaaagttatatagcactcattttatgctaagcatgaagcacttcattagtattaactcatggaatgccctggaggtaggtgttactattatctgcatttgcagatggggaaactgaggcacagaaccttcaatagcttcctccaggtcatggagagtgagtggctgagcctgagtttggatctagagtctagtctcagaggtcaggggctgaaccgctgcccagtgctgcctgctgctgtttgagcacctgctttgtgccagcaactgggaaatattcttctgtggccatgtcacagacagtggacactggtctggccaggtccgaagcccagagcctcctccaggttcttgttccctcattgggaacatggtcttgtggacggataatcttgaaccccgtttcttaaaatattctttggtcatttaaagccaagcgggaaggggtggaaaaaagccctttatttgctcaggaaggtgcatgttaaagaaaaaaaaatcgtccaatgagacttgttaaaatggtgaagcgaactttattcaggaccacggaggcaggtacagggaccacagccagcagattttgcagtgggaagaagagactgggatccgcttgagtacagtatgggcaagtgggagttaatttataaccaaggatcagggtggggttggtgggtggaaaaatcacaaagagaaaacatcaagggtaagggggattctggctaaaccaaccaaacaggattcttgctgaagacaggctgggtgatcagacatcacctgtggcacggtggaggctgaggaacccgatcagatatccagagtgatcagataccgagggtgggaggttctggctaaactgactgagcaaggttttttgctaaaactggattttacaaggaaatgcacagatggcctagaaggttcaggagcttgatataagtttgatcaagcgaagaatctttgtccatggagagtgatgtcagaatgcggctttactatacagcatttcgtgttgacggcatctctagctattattgttaactgttcacaaaggacaacaagcaaaatggcccacagctttcttttcattactgctaatcttagcagtggtcaaaatggttggctctgaatttctttcttttttgggtttggggtagtaaatggagtgactgtggacatgttttatggattattttaagcgtatgtatctattaaaaattgagagggcccacatggcctgctttaaaaagtcaactgtgagctgtatttggtgctgagccctgaaccacccgctgggcagtgggtggctactggcagtgcctgtggacgttctcggggaaagacggtctgattgcacctgctcttcctcagttcctagtactgtgaggcactctttccccactctcccacaaccctcagcttaacacaactgtggtgattgctttgtccgttttcccatgaggaccctgagatttcaacaaggttcagtaactcacacatcgacacttccagggagctgtgttgccaggaactggcccaggtctttccctcctgggcccatcttgtccttaatctctgctgacattgatgggtttagttaccttgtcgtgtttcttcagtgttttatgccatacagtctcccttgagtagatgttctgtataggggatatcacagcttcctggtgagaactgggttaagtccagcaggatgcctgggcttagtggcactggagttgtgggtggccttgagccccaggcagtcccctgagagcccttcccgatggatgtttgagtcactgagtgtgttccttcttctttctggtcaggctgtcctggccgctgcacttacaattgcaccccatcctcctacccactctgatcccagaggagagtgcttctgctgtaacctctaaggcacaagtgggtcccaaagtccttaaatatggagggatgtggggaagcagtggtgaagatgcaggccaaaggtcagcaggcaacactggtctttgttgtaggtaaccttcatctagagattcttctagttctttccagatttatcttctaaaaactaactggtatggaaatattacagtcctgtaattctttcttctaggtcatattgaacattccagatacctatcattactcgatgctgttgataacagcaagatggctttgaactcagtaagtggttaattattaccttcctggccttttctttgttcccttgtcccttctgttattcccaacattgtttggggtcagcaattgcaagggctatttgtagggaacgccgggccacgctgatgctgttcaaggcaccctctagaaagagtgagtcaggtgcgcagccacacagctgcccaggtgagtcgcaagagccatggctgttgggtacacgtgggtgatatgcattctcaccttgacctcatcccttcctgctttgcagggcagggggcagggacaaggagtggggacagatgaggcaggatggcgataacaggggtatgttccagggaagagacccacgtgggatcccatcgatgcccaactgaaggactaggaaaagtcataactgcatgaggcatgggcgggccttccctctccctggagcccactccctttggagacaaatgtggctaatctctgctccccattgtaactggcccgtttgccttatacctaaaataagtgcatttagtaaagagttttaaacacttcctagattctatatctggaaaatagaccttgttttcccctttatttgttactgatattcattttttactgggtctgtgtttaatttcaagtccactgctcaggaattttcagggacaaacctgcttgctagtggatggcaagttcttagatggctgagattgcagcctctgactccctcatttagatgagagattaccttgtgttttccctctgtgtgctgctctggttcagagctgtaatcttcggacttttgaaggacagcattagtgagttattaagtggaggtgaactctgcgggccttgtttggctcattccattgtaatcaacttccctggaggcctcatttaaagttctaagagctatgctgcctctgctcagaacacctgtccattgagtacaaacttgctgtcattctctgaggtcaataagagacaaggggcaggctgggcgtggtggctcatgcctgtcatccagccctttgggaggccgaggcaggcagatcacctgaggttgggagttcgagaccagcctgacaaatatggagaaaccccgtctctactaaaaatacaaaaattagccgggtgtggtggtgcatgcctgtaatcccagctactcgggcggctgaggcagacgaatcacttgaacctgggaggcagaggttgcggtgagccaagatcgcaccactgcactccagcctgggcaacgagagggaaacactgtctcaaaaaaaaaaaaaaaaaaaaaaaaaaaaggagagagagacaagaggcagaaattaatgaataggaaaaagttttgcatgggggcccttagatacagataagaggaaaacgtggcctcccagagtggcccagccaacaggtaaccggggagagctgaaagaaggggaagatgtgggctggtggggcccagagtggcctgtttggggcgagtcttgacagggtaggtctcgggaaggcaatttttaagtaggctgaggaaggggaggtgggcattctaagaagataggcaccagctgaaggatggagctgagaataaaatgctacttctaggcagttcctttcacatttatgtatggccacactctgacccagaaatttcatttaaagggatttagctacacctttctaccctctgcacaaaaatgcatgtaatgtgtcagaatgtttattgcagcagagctgctaacagcccatgttgtgaaccactcaaatttccattaatagcagatggtgagtgaatgacgatacattccttatatgagagaagaggatgtagaagaggtatttgcaggataacacagagactatgctcctttaggaacatttcttcactcagtttttttctctgcaaaatgtttgtattaattcatacctgtgccagtctcgctggcagcgggagggattatggtgagcgctcaaggagattatgagtacaggagcctggcggcagctgagttctccaatgttagctattattcttctcattataattatgatattgctagtttacagttttgtatatcaatattcaaggaaaagactggaggaactaaaagtacaggattttggcttaatggattctaactccaaacctctacacaactttttaaaattattactatattttactaaatcccaatgtctgccacttttttttttttttaattttaaagaatacatagcccattgggcaaacttgggtttgcatgcttggcttttcagcagatgcagaagccctggggtgggtttgccctggcttgtgtggttctcgagtttgagtctgtacgtcttgagtgggtcaggggccccagagctttgctgccattgatttctccccaacagcagacaaagtctcacaatgacctcaattttccctctttctttcttttgaaggacccatttttcatggtacttgtcttgactgttttcatagtggttttttttttttttttgagacgggagtttcgctcttgtcgcccaggctggagtgtagtgacgtgatctccattcactgcaacttccgcctcccgggttcaagcaattctcccgcctcggcctcccaagcagctggaactataggcgtgcaccaccatatccggctaattttttgtatttttagtacagatggggtttcaccatgttggccaggctggtctcaaactcctgacctcaggtgatccacctgctttagccacccaaagtgctgggattacaggagtgacccactgcgcccagccaaatctgtatttttgtatgtagagggaaataattttctcaactgtaatcgtgaataaatattgtattaaattgtcatgaaggaactcctgtttaataaatagacatgtaagaaaaaaagaatgaatagtaaaaccaaaccaaaacaaaaaactttggtaattgaaagttctggttagtttttattctaaataagttcaatgtttgacatagttgtagacacctagggagaatctgtagtacctcttcttttctattacttttttttttttttttgagacatagtcttgctctgtcgccaggctggagtacagtggtgagatctcagctcactgcaatctctgcctcccaggttcaagccattctcctgcctcagcctcccgagtacctgggattataggcgcccaccatcatacccagctaatttttgtatttttagtagagacagggatccaccatgttggccacgatggtctcgatctcctgacctcatgatccgcccgcctcggcctcccaaagtgctgggattacaggcatgagccaccgcacccagccttgtagtacctttttaatatcttgtcactttcttattaatctctaactgtacaagttgtatagacaggatgcatatttcacaaaataaaataaaaacattatcaatttaaaagatcattggtatagctgtggtatgatgtgatagtttgtatgtaaccctttagtgcaaataagagttttatattattcatcttcttttcaactgtttaggggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaagtaggattctctttgtgtttatttcctgtaataaacccggggtcccgggtctacctccccagtgggctctctgctcccaacaccactgtcgccacctctcctggtctgacccttctcctccgtcttccggagccagcctgtcttcatgcctttccactctcttcctgctgggtcaaagctgtcgctgccaggcctagcctcttgcccagggcctgcagatacctccctcttccgttaactctcctgtacagctttgcctgttctactcagcgtaggcttcgagatcaccttctctgggtttcttggctgtgtggtttattggtggctgctcttgaataaatggtaagcttccaagagcagaaatcaactatcttacctttgtcatacagtcccctggatatgacaggtcatgtttaagatgctctctgttactttgtgtttagtagatgacctggctggtgcggttgtcacacagaagtatctgagctgtctcattgggcttcagggtcctgcgggtggccggagcaggccagagcgagcgtgggaggactggaggttaaattttatttgctgtcacttcagtccctcctcttcctcaccctaccattcatctcctttctttgccatttcgtttggtctcatgtctttccagtccgcccctcttgcactgcctcccagctgttaattcaggctagtttcagtgctaagaattgtccaggtggaacccgcacagcagtggctacgccaaagatcttttacattgatcttttaattgatagtgcttttattttattttgtgaaatgtgcatcctgtctctacaacttgtacagttagagattcgtcagaatgtgaatttccatgtgtccacatcacataatcagtcatcaacgtgattaatgccagtgctctgtgaagccctgtctgtccccacctctcccccactcaggacaagtgctgccgctcctgcgtttctacccctcctctcaccccctcacccttcacaaatggctcttccaccctgttcctctccagccacctcctgtctctgagttttctaccccttcatgagccttaattcctttctctcccttctctcattctctctttcccttcttccgatgctccccaccaagcagatacttgtgtttgcactgatgttcttctgggggtggttgggtaggtcaaggccagtggttctcagtcaggacaatttggacccccagagacaattggcggtgtctggagacagtgtgggtggttacaagtggggagggggtgctgctcctaggtaggcaccggggatgctgctcaacctcccgcaaggcacaagacagccccccgacagcaaagaatcctccaggtaaaatatcgcttagtgtggaggttgggaactcctgatctttgtccaccgtggggatggaaggaagccagggtgaaggggataaggaactgtctgtgtgagctacccccctaagaaatggcagtgaaggccctctcctgaagtcagcatctccagacggtggtgttcccaccagtgatggctaaacccaggaaggcagacgaacccctggcccacatgatggacacatttccacgtttcctgggaaagactagatgatgacagagttggacagggatatatcagtcccttatcagttgcttttataagggaagaatatgaacctacatgtatgatttttgcattataaaatttataaactcagaaataatttagtaatttaattgaacttaccctggtactatttttaagaaaacactgttggcatctaatatatagaaatgtatacattttgaaagtgtaatgtttggctttgaaactcttctcccagttattgagtagcaccaattcagaagcacccatcaggagtataaggaatttttctagacttaatgtatttatttgctttagcaacagccgtagaaggttgatgattgagtaggagcgaacacacagcttaaaccttaggctttggagaatcgaaggtccatagtcctccaaatgtgcgtgtgaaacaagcagctaaacgtgagaaaccatgaggctgagttgacaaccttaagttggtcatgattgagggaaatggatagtgggttttgtgtgcgtgggttttttttgttgtttgtttgtttgtttgttttttgagacaaagtctcgctctgtcacccaggctggagtgcagtggcacgatctcagctcactgcaacctctgcctctcaggttcaagcgattctcctgcctcagcctcctgagtagctaggactacaggtgcactactgcttctggctaatttttgtatttttagtagagacggggttttgccatgctggccaggctggtctcgaactcctgacatcaggtgatcctcctgccttggcctcccaaagtgctgagatccactcccaaagtgcatgggtggtatgagccactgtgccgggccagtgagttgtaattaaactgtgcaacagcagccgtgacaagccaagacccagtggtgtctgtactgaccacgcgctgactacagatgcctgtccagcaaagtaaacttgagggcttcccgctttacacactctcctggtgagaactgtacagattggcacatgctcagtgtgaggttcaggatacctaactgaatagccctcatctcagcagaccagttattttatcttggataaaatggcactcgggaagccatagacggaggaaagtgtccttggtaatgggccagagagggcatctggaatttctctcatgaactaccttggccccagcttgcctccatcatatataatcattgcagccgagttatgagaacatcgtgtgcctgggcatggccctcagaccagtggtgctggcctcatctgtgagcctgttagaaattcagattcaggtcgggtgcagtggctcacacctgtaatcccagcactttgagaggctgaggcgagtggatcacttgggcccaggagttcgagaccagcctgggaaacatggtgaaacctcatctaccaaaaatgggccaggcatggtggtgcacatctgtggtcccagctacttgagaggttgaagtggaaagatggcttgagctcaggaggtggaggctgcagtgagccatgattgtgccactgcattccagcccgggcaacaagcaagaccctgtctcaaaaaaaaaaaaaaaaaaaaaaaatgcaatgcagattcatgcccgacccccaactgaaacagaatctctggtgatgggaaccaccacattccccaggtaattcttaagcctgtaacagtttgagagtgatggtagaattatcagaatgttgtcacaaagagagaggtatggcatcgagccctcagcagatgggtcagctctgagcttatcgccctggcagacctccggaggcacaggtgtggccaggtggggagttccaggctgccacaccacctctccagcctgggagggtctgttcaaatgccttctcctcccctctcctccacctctgcacctttcttcagggaaagctcctgggagctcctggacctggggctgctgggcatcactggggtgatggggatggatgccctccctcctggcccatgagctggctgagcccagcgtctgactctgccatgcctctctggcacctgtagctgtcttctgaactctgtgtgacttttggcttaccctctgaccaataatgctatgtctcactagagaaaaatcagtgaccttttccaaattcgttttttttctgaaatgtgcctttcctctgagtttggtatttctgcacttggaccaaaaggcctgcactggcgctgtccagcaggacttagggacaccatgggaaggttctacagctgcactgtctgacatggtagccaccagccacatatgatgactgagcacttgaatttacatcattttacttaattccatgggacattaattaaatagccacatgtagctttggtggatgtccgaatgggacagtagcatccattcgctaaatagccttgggtaagacaatttattctgctttgagtaccactaacagagtgatgatagtttttaggacagtattattattttgcttatgcttgtaattaccaagatgaggtcttagttctttttttttttttacattaatcatctgcctattttaagaccctcttttagcatactatctttttttttttttttgacagagtttttgctctgtcgcccaggctggagtgcagtggtgtgatctcggctcactgcaacctctgcctcctgggttcaagtgattctcctgcctcagcctcccaagtagctaggattacaggcacctgccaccacgcccagctaatttttgtatttttagtagagacgaggtttcaccatgttggccaggctggtcttgaactcttgacctcaagtgatccacccacctcagcctctcaaagtgttaggattacctgcatgagccactgcgcccagcgaagcataccatactttttaaaagagcctaacctcggtgtactttgctgaagatcacagcttcctcctgttggtctccaaattgcataaggtttagtgttatggaagaagtaggatcgtccatgatatttcttaaaattaaaagtcacatttttaagtgtatatgaaataattatttcttaatgtggtgtactataaaaataatagataacatttgagagcatgacttctaaacaactctggtctgaacgttgcactctcctccttcttccagagactaagaaagcactgtgcatcaccttgaccctggggaccttcctcgtgggagctgcgctggccgctggcctactctggaagttcagtaagtgcagggagcctcgatcccaccatgtgctcctgcagtccccagtgctctgagccagaccctgctctctgggctattgagacctctggaggccctccgtgaggttcctctcttacataacgaggctgtctctcttcccttctcttgtttagctatgagattgacacatcatggggaaagcatttagaatgtacccagtgctttggggtgcttggtgccacccagcactgtgagcacaggttcttctaccttggggccacacccagttacctgtatctcactgcacagcagtggctgttggggaccaggcccacccctccatgtcccacctcctgcaactgcagcctgagccttcccatcagcctggggtggtgcagacccatgtgccattgtggatccttcaagttacctgtgtggcagagaggacgtgtgagtgccgtccaaacccaaacactgagagggtccttcccattgcccccacggaagtaaggtgccccagtgctaattccacttatacttgctggtggcaaggacacttctcctccttattaaagtgggggattggctgggtgaggtggctcacgcctgttatcccagcactttaagaggccaaggcaggtggaccacctgaggtcaggagtttgagaccacaagcctggccaacatgttgaaactccatctctactaaaaatacaaaaattagtcaggcgtggtggcgtgcacctgtaatcccagctacttaggaggctggggcaggaggatcacttgaacccaggagttggaggttgcagtgagccaagattgtgcccctgcactccagcctgggtgacagaatgagacttcatctcaaaaacaaaacaaaacaaaacacagtggggccaggagttggaggctgcagcgagctacagtaatgccacggtgttcctcactccatgaggctcattgcgtttctcagcctgaagggcacctctcttctgttttctctgcaagtgggcagcaagtgctccaactctgggatagagtgcgactcctcaggtacctgcatcaacccctctaactggtgtgatggcgtgtcacactgccccggcggggaggacgagaatcggtgtggtgagtcagccttgaccttgggaagggactcctctgctcaccttggagacagcagccgggtccaggggcctttgggtgactgggcctggcgtgcgtccagtacgctgacacatgatgtcattgaatccctgctccaggctgagccctggggctcagagaggttgtgtttccggcccaacctcacccagcaggtgggagatgacagggccaccgaggactgtgtcattggaaccacacgtgctctgaactgccacaggaagtcagttaagatgagcaaactgtttataaagttggagatgcaggctaggaacggtggctcatgcctgtaatcccagcactttgggaggccgaggcagatggatcacctgaggtcaggagtttgagaccagcctgaccaatatggtgaaaccttatctccactaaaaatacaaaaattagccaagcgcggtggcgggtgcctgtaattccagctattcaggaggctgaggcaggagaatcacttgaacctgggaggcggaggttgcagtgagctgagatcacgccactgcattccagcctgggagacagagctggctcaaaaaataaattaattaattaaaaacaaaattggagatgcactatgttattttcaaaacaagctgcctttaaagatctatctgttgtcacagggtgggctcatctgtttcattttattttctgtggtttatctatttattcattttaatgaactaggaagcattgctcctatttatggcataccacatgatgtttggatacgtgtatgcctgtggcatggctaagtcaagctagaacatgggccttacctcatatacgtgtcttattaagaacacataaaacctactcttgtagtgattttcaaatatgcaacatatagtttattaactgcagtcactatgatgtacaatagattgctcgaacttattcctcctgtctaactaagattttgtgacctctgaccaacatctccccagtgttgtcaccccccgcccccagcctctgatagctgcctttctactctctgcttctgtgagtttgatgtttatacattccacatgtaagtggcctcatgcagtgtttctgtctctgtgtctggcttgttcacttagcgtaatgtcctccagcttcatctatgttgttggaaatgacaggatttccttctttcttgtggctgaatagtattgccttgtgcatatacaccacattttctttatcccttcattcactgatggactcttaggttgatgtcatgtcttggctgttgtgaaaaatgccgcagtgagcgtgggcgtgcaggtccctcttcaacacacggatttcctttcctttggatataaacccagcagtgagattgctggatcacatggcagttctgtttctcaccttttgaggaaactccatactgttttccataatggctgtagcaacttccactcccacccccacggtgcaaagtctccatttctcttctacaacctcaccaactcctgttattttccatctttctgatagtagccatttgaagaggtatgagatgatacctcattgtggttttcatttgcatttttatttgtatttttcatgaatttttgagggtgatttcaagggtagttagtgactcgaacagggaaacgatcctgagtatgagggttgtgctaatcatccccctcctgccagctgcgtacggaatggggctctgcagatggcagggagctggctcgtttctctttaagagctgccttttacttttcttcctcttcctttaaaacttatttcctggccggacgcagtggctcatgcctgtaatcccagcactttgggaggccgaggtgggcggatcacgaggtcaggaattccagaccagcctggccaacatggtgaaaccccgtctctactaaaaatacaaaaattagccagacgtggtggtgcgggcctatagtcccagctactcgggaggctgaggcaggagaatcacttgaacctgggaggagggggttgcagtgagccgagattgcgccactgcactccagcctgggcgacagagccagactccatctcaaaaaacaaaaaaaagttatttcccaagcacagccatgtattccaggcttgtggatcagcgttggtggtggtgtgtgctctcatatcttagttccagctaagcacactctgacatgtttacactagaaccatttgttttttctagaaatagaaatttcagaattgtagagtcagaggacttaccagaaatctcttaggtagttctcctcccctccctcaagtgcagtcctaacctcctggagttttctgtagaaaccacaagcctcagagctggccgagaattctagccaaagatttttccatgccaaagtaatcccccctctcctaagggccatccttggtggggactggtttcctgttaagccctcgctgtcagtcctggctgtggaatttcctggtgaggagcactggcccgtggagctcggccctcgtgccggccttgagcaggcccaagtgttccgtgttcttgatacctttcctccagcacagtcttgcttcccagaaaaaggtttgcacttgaaaatgatgcatttgctgattaaacatagttcttttgctttatttggtttctaaaataaagtgggagtttttgagattgagtagcgtgaggttaagatagcacgtggaatggctttttcttttctttctattttttttttttttttcctggagacagggtttcactctgttgcccaggctggagtgcagaggcatgaccatggctcactgcaacttcgatgtcctggggttaagcgatcccccagcctcagccccccaagtggctgggactacaggtgctcgccaccacacctggctaatttttgtattttttgtagaaaatgggtttcatcaatgttgtccagactggtctcgaactcctgacctcaagcaattctcctgcctcagcctcccagactgctgggattacaggcgtgaactaccacgcctggcctggaatggcttttgatgttctcctatgtgcacatgtgggtgaataaacaccaacaaagtccttatgttacctgaagagttgctctcttcttaatatttaagtcgtatttatttaaatactttaatagttgtacactattaaagtattattaggtcaaaatcaaggaagtacaaaagggtatgctgtgaaaaatctcttcttccttgctctgcttacttacctaccccgcatccccccatacaccccagacacacacacacacacacacacacacacacacacacgcatcactcccatacatgcccacctgtttaccagccaatcacatttcttggggcaactcatctgagttgcttctctttccagagagtttttgcataaagaagcacaggtatttctgcgttaccatgaccctatttcccagtggttcctagccagttgactctcctgcactggataccatcctggacagcattccttagggaaatgagccccctgttttttcccaccatggcacagttggtcctttgcatggacgcaccattattgcccctgtctcttcttggtggaccttaaggttttctccatccttttgctgtaacacacactgctccaagtgtgtgagcatatcagtaggaaacgcttccaggagtagaactgctaggtcagagggcgtgtggatctgtaacctgacagacctagaccggcttcagtttggttttatccagtttccatattgattattcatataaaaggaaacagacaaacataacgctgtgcatgtattctctcttagaccagaacaggcatagggtgcacttttaatttgtccatttcgtagagtagaaattgtttttgctgaaatgaacaccttaggatgctgaagaatatgacccgtcccatggaaaacattcaaaaatgtgtgtagcgctttcttcccaagggtgtgtgtgcgcatattttaacactaattcactttctacttccgttgctatcctttctgtgagtctttctcagaatctcagaaaagaaactaaattgttcactctagttatcaatgctgtactctatacctggaatttgctaaaagggcagattttaagtattctcaccacagaaaagagaaaagaaaatggtaattatgtgacgtggtggacatgttaactagctttattatggtgagcatttcacagcggatatccagtcatcacgctgtacacattaaacatgtacaattgggtttttttgagacaaggtctccttctgtcacccagtctggagtgcagtggctcagtcatggctcattgcagcctcgacctcctgggctcaatccatccttccccctcagcctcctgaaaagctggggccacaggcatgtaccatcatgccaggctaatgcatatatatttatattttttggtggagatggggttggtctcgaactctgggctcaagtgatcctcccgccttgcccttccaaagtgctgagattacaggcatgaaccacagcaccaggcctacatgtaaaatttttatttgtcaactatactttgacaaagctgagaaaaaaaatcctaatatttaaaaaaaaaaaaaaaaggactagcttgagaccttttccagctctctggcttatcagctgccgtctcttccgggtgcagatagctggaagggaaagaaaatccctaaaattacccacaagccaagaatgaagtgtctccctttgagccacagtggcagttttgtttttaatcatagaagtgtattttgagccgggtgtgctggctcacgcctgtaatccccgcactttgggaggccgaggtggggggcggagggggtggggatcgcctgaggtcaggagttcgagaccagcctgaccaacatggagaaaccccgtctctactaaaaatacaaaattagccggcgtggtggtgcatgcctgtaatcccagctactcatgaggctgagtcaggagaatctcttgaacccaggaggtggaggttgcggtgagctgagatcatgccattgcactccagcctgggaacaagaaaaaaaaagaagaagaagaagaagtgtattcatttcagttacttttaaaaaagtgaacagactttatattttagagcggttttaggtttacagaaaatgaaacagacagggcagcgagctccttgtactcctccccagcacacagttgccctgttatgaacatcccacatcagtgctgtgcgttcattaacaccgatgaacctgatgcatacattatgatgaactgaagtcctggacttcaccctttctcttgtacagttctgtgggatttgacaaatgcataatgctgtacagccacaatgatagtatcgtccagagtagttctcctgccttaaaacctcttttgctgcacctgtttctctctccccactcaccccagctatctgatcttcttagtgcctccgaagttttggtcttttcaggatgttgtagcgttggaatcatggagtatgtagccttcaccacatacaccttccttcactttgttggcttcctttacttagtaatatgcattcaagtttcctccatgccttttcatggcttgatagctcatttctttttagcaccaaataatattccgttgtccagatgtagcacaatgtttatccattcatgtaacctgtgaccgactcacagataggatgtggaatcactcaccacagaggcattagacaataatcagacccaagtcatttcatgggggaacaagcccacaggtaccagactgtccagtgagtcagggccactcgtaggaagtaagaagagaggctagagcatagccaggtcctcactttatactttaagcccatgtgtatttctcccaaaccacacagcattgtttccatgctttcagctttgcatgaataacgtgatacttgaacgcatcatttatcacttgctctctttcccacagcgctgttttcaagcttcttcctgttcatgatgctctgcttaacccttaagctgcatgggattctgttctgtgaatacgcccaccccatgtattatcctgcccagcaaaaagtccccaaaactctggatggtggttacctctagggagggagagaagagattgggaatagggagcgacttcaacggtgtttgtaatgttttgtttctttaaataaaagagctgagatcatttcagcagaatgttgatttagagtctcctggacaatttgttgctcaaagtgctctcttaaagagcactttaaaaaaaaaaaccttttatcttattatttatttatttatttattgagacggagttttgctctgtcacccaggctggagtggagtggtgtgatctcagctcactgcaacctttacctcctgggttcaagcaattcccctgcctcagcctcccaagtaggtgggattacagatgcgtgccaccacacttggctaatttttgcattttagtagagatcggtttctccatgttggccaggctgatctcaaacgcctgacctcaggtgatctgcccgccttggcctcccaaagtgctggtattacaggcgtgagctaccatgcctggcttatcttatatatttttaaaaacagcttattgagatctaatttatgtaccataaaattcaagtatataattcagtgcttttatatataaaacatatatatgaaatagcttattgagatataattttttatataaaacagcttattgatatgtaatgtatgtaccataaaatttaaatatataattcactggcttttatatattcacgaatatgtgcaactatcaccacagtcaattttagcatattttcatcagctcataaagaaaccccaagcccttgaactatcaccccatatccctcctcccagcccgtccctcctactcataagcaaccactaatctacttagtgtctatagatttcctactctaggcattccatgtgagcgggatcatgcaatacgtgggctcacacaatataagtggcattccatgtgagtcggctcatgcagtatgtccggctcctttcactgagcataaggtcttcagcactcatccaggttgcagcctgtgtctgaatttcattccctcttctggctgaatcgtattccattgtgtatcttggacatatcctattctgctcacccagccgttggtgggcgtttggagtgttttcgcctttcagctgttttaagagggttgcagtgaacatttgtacaagttttggacccaatgcctgttttcaattctcttgtgtagagagcactttttagcagaaaaagaatagatttgtggcctccctttgtgtgcggtcagtgccttgagaagagtgaactgtgctgccacctccggagccgtggagagcgcggggcttgggtagcagctaggacgatacaagttgggacaaggccaggtgcaatggctcacgcctgtaattccaacactttgggagaccgaggcagggggatcacctgaggtcaggagttcaagaccagcctggccaacatggtgaaaccccatctctaataaaacagaaaaattaactggacggggtggtggacgcctgtaatcccagctactcgggaggctgaggcaggagaatcacttgaacctgggaggcggaggctgcagtgagtggagatcagaccactgcacttcagcctaggtgacagagcgagactccgtctcaaaaaaaagaaaaaaaaagaaagaaactcatggataatcctccctctcgtgcagttcgcctctacggaccaaacttcatccttcaggtgtactcatctcagaggaagtcctggcaccctgtgtgccaagacgactggaacgagaactacgggcgggcggcctgcagggacatgggctataagtgagtatggggcagcacccgccgagtgacagtaacagacagcagaaacacgagaagaccctctctctgcctccctgtgaaagcaccggcacatgagtgctggggacaattgtcaccttccaaaagctgagccctataaccagcaggtggaatttgtcctgctagggctgtgcccagcacacagaccttggctcactgccaccttgccctgcctcctccttggcctctatagactcctggttgctcgggagtgcccagtgctgtggtcatctggtcagaggggtaggctgagggcgttaggtgcctctttttccaaggtgcctctcagccagggtccattcacctccctgggtagaggttggaccagaacagctggcgaggagggttgggctggggagagcagcagagacaaatcctgtgccagtttcacttcattcgggagccatggaagccttttgagctggggagagaatcaatcaatcagactgatacttaaaaaatgtcattcctgctcgtagctctgagggaaggtgggaaggcttaacagggtgtgtgtcgcctgacagtgattcctaacgggggtggggcggtggttaccatttaccagcactgcctggggagatgcggcagccctcaggcatcgggggagagggtggtaggatgctactgccactttgttttccatgggagggtccccaggtgatttctatgcaactttagggtattcaatatgccagttttcagaatgaattaccactcggtgagaaagttggcatcttagctagtcactgtgacatccctaaacagcaggggtgaattacacagcaaagcccccccatcacagtccaggaacctggtggaattgataactggggccatgttaacatctgtaccttttattagattaaatgtgtgtatgattatacaatcctatgtccttctcatagtttcttgatcctaacctggataagaaacacgaccaatgaaggaattttgtctgacactttagggttattgaatcgaaaaatcgttacaatattctagcacttggttagaacgtgtgattttttttcctaaatgctaaggtttttccctcttattctgaatgtcgtatgagcggtattatgacatagtataggatttgtgtttgcttatgccttaaccattatcacaaataaggttttcttttttaggaataatttttactctagccaaggaatagtggatgacagcggatccaccagctttatgaaactgaacacaagtgccggcaatgtcgatatctataaaaaactgtaccacaggtatgcagcaatttcttcttgaaaaattttggaatgaaatcaactaggagacaccatggggaatcgttgtcctgagtctgatttctctgagctgcaatactcggtctggatgggttttgcattgggaggagattagagtctgaccaggcctggttactctaagcagcccttggtttattcataggaagtggctgaggtttctctgctatttcattttcagcctctaccgtctgcccttgttggtagcggctcacacttgcaacatcgacattcaactctatttagttttctttcctcttcagacatttagaggtgtacctattttgtcagggcgtggttctaggaatccaagataatgtctcagtgtcccagccagggtgaccggctcattccagtttgccagggacttcactggcttgagcaagggaagtcctgctccattccaggcagctgggctggctggtcccgttagccccaaccccgggacagcagtgccagagggtgctctgtgagggatgggcagcattctggcggcctgggaatgagttgtggtgtttccagggggtagaagtgggtacaagccacaggtcacatgatgagtggctgacctggctgggagggcagaagaggggatggacttaggctcttccttttgctttgcacatatttaggatgtttgcagacttgctatgattgttgctgttatgtgttttctgatgtgaaagatacacagtgtcctttgcccatgagctctccttgcctcccaggtccccagggcttatgcctggtgtctaggcatcacctccctgcctgccaggtgccaggtgctgcatttcgggggaggatgaactaatcaccccgcgccacctttcctctgagtgggagcctggggcaggtttgcattcctggaggccgctggtggaggggtctgggggcctgacttccactgcagcctgctgtcctggggaatgtggcagggcaagcccagtggggagggctgtgcacggccaggtgcacccatcaaaacagcagggctgcggtttgtccctgtggagaagctaaacacagctgcctgggcactttgtaaatgctgagtggttctttgtctttctgggttacacacggaatcagggagccaagtccagccgggcagggacggggggaggggaggaggtgctgccgtcccttggcaagagccttgggaactcacaaggaggctggagggcttggaagaaagaagagaaggccattgtctggtaggctctattctatctcggtggtggtggtggggggaggcgcacttcttttcctctttctgtgcagcagttgccctttgatgcctgagttcttggcttgttttctgtcgggcttctgtgaataaccacatgtgccctggcgctgtgaccacacagggctatccctaccgaccttaggattcttaggaaatgtcttctcttaaaggggacatgtcttcacttggccgtgtcagtgccccagagccagagtccacctggaatgcacctgtagtcactgagaacccggggggtgtgccttagtaagaaggtgtcaggaaggacctattattgtagggcctgggctcctgcaaggtggtttgggggtggttggaggaagcagagatttgctctggattggatgctgtcaggaagcaggggtaattctgtgaggctgctttattattttttttctaggaggaggttggaatgaggctaggctaaagctgtgattggtaaagaaacgtccgtcgctcaagttagccaggacaggaggagacatcagatcgtgattttgtggttgtgagcacaaggttcctgttctgtctgttcagacatcatttcggaggaggctccttgtgtcttgccccatctcaggcatggaggggcctagtccgatattgacgctcagtgaaataattcaggttccgcagagcacacggcccagctatcagggcgggccagctctgcatgccaggggccgcgtcttcccttctcagcatagcctgggaaattcactgcaggacaaaatgcatcagttacttcctcttcatccataacctgggatgtttgactcccaaatgagtaactcttacgtttcttctaatcctagggaaactattggttatattgctttcaacactacaaatttaaagcagttataggagcccagaggtttccaaatggcttccttaaaaattagaagatgattttaaattccaagaggaaaaacaaaactagcattattgtatacttaccctcacaaccgtcctaggagctggtacaattttaagagaggttaagtaacttgcccaaggtcacactgtggggatgtgagccgcgtaccttggctcagtgtctggtctttgccactgtccctatatggatttacttaccttattggagttgtaactagcagacccttctatgtctcagaagacaggagagggaacatcggaagaaatgactgatttctaagcatgtgagaggcaggtgactccgcactatcgtgaccagaatttcccctgttctttttgcagtgatgcctgttcttcaaaagcagtggtttctttacgctgtataggtaagttcatctggagtcccccttttgatacttctaactaggaaaagctctctactttcagaacagtactccctgtgtctctgggggcgtgggagggaagaaggtggggtcacgggttggaatgtgcccagcggcgtctcgctctttccaaggagctcctggtttagatttccatggcctgtagacaccttcagccttgggtccaagggacaccccctgagatcaggcacgctcaagaagctgacaaagccctacactttatgccacccatgagctggaggcccggcaggtctctttctccagaaagcaaaggggggtggcgttagtgagccctggcagccacctaacgtggacttggagcatctgcggggctgtggtccagcaccaccgtgtggccaccaggtgctcatcagccagtgggacccgggaggagggacaagaccagagaacaacagtgctcttgcctcttctctcctgaattttggacggtggcttagacttgggtgtccccatctctgtgtttagagtgcttacagtttccaaactgtttgcaaatgtggaagccaccgtccctctcctctgggatggcccagtgctgtcgtggggccgtggtcctgagctcagcttttcatttgaagaggtggaaggagctgacaccgtcccatcccggcagggctggctcaggtcttctttaggtcctgagtgggggtccagcacagccccaagggtgcgtggcacccgccctgccctctgcccatgcactcatctcctggtggagaagacactcacacacaggaagcagggaaggcagcagacctcactcacccctcaccccctcactcaccccctactcaccccctcaacctctcattcaccacccaccccctcgccccctcactcaccccctcactccctcaaccctcactcacctcctcactccctcaaccctcactcacctcctcacctcctcactctccccctcatccctccctcaccccaccccgtcacctcctcactcacctcctcaccccctcactcacccttcaccccctcactcaccacctcacctcctcactcaccccctactcaacccctcattcacccctcaccccctcactcacccctgcaccccctcactcaccccttcatccactcacccacctgctcacctcctcactcaacccctcaccccctcactaatccctcactccctcaccccctcacgccctcactcacaccttcacctcctcactcaccccctcaccccctcaaccccttacttaccccctcactcatcccttcacccctcactcaccccctctctcacccattcaccccctcactcatgccttcaccccctcactcacctcctcactcacaccttcacccctcagtcaccccctcactcaccccttcaccccctcaatcatgccttcactccctcactcaccccttcaccctctgaattactccctcatcccctcactcaccccctcactcaccccttcaccccctcacccaccacctcacccacccctcacccaccccctcacctccttacccctcacccccctcactcacccctcaccccctcactcaccacctcacccacccctcacccaccccctcactcactccctcatcccctcactcaccccctcaccccctcactcaccccctcacccacccctcacccaccccctcaccccctcactcaccccttcaccccctcactcaccccctcactcaccccttcaccccctcactcaccacctcacccacccctcacccaccccctcactcactccctcaccccctcactcaccccctcaccccctcactcaccccctcatctcctcactcaccccctcacctcctcactcacccgctcacctcctcactcaccccctcgccccctcactcacccctcaccccctcaccccctcactcacccctcaccccctcgccccctcactcaccccctcgccccctcactcacccctcaccccctcaccccctcactcatcccctcacctcctcactcaccccctcacctcctcactcaccccctcacctcctcactcaccccctcacctcctcacccaccccctcactcactccctcaccccctcaccccctcactcaccccctcacctcctcactcaccccctcacctcctcacccaccccctcactcactccctcaccccctcaccccctcactcaccccctcacctcctcactcaccccctcacctcctcactcaccccctcacctcctcactcatgccctcaccccctcactcaccctttcacctccttgctcatcccctcacttaccccctcacttcgtcaatcacccccccacctcgtcaatcaccccctcaccttttcactcaccccctcactcacccccttacttcctcacttacctcctcaccccccactcaccccctcaccccccactcaccccctcaccccacactcaccccctcaccccccactcaccccctcacccctctcacctcctcactcaccccctcacctcctcacttatcccctcaccccctcaattaccccctcaccccctcaattactccctcatcctttcaattacccactcaccccctcacctcctcactcctcactcactccctcactcaccccttcaccttctcactcacctcctcgtctcctcaccccctcactcacttccagccctgcccctcccatcttccttttctttgtgtgagaatctggggtccctgagtggtgtcagtccctccaagactcaaggagtccccagggccttgttatccagaacacccccacctgggtcccgggagaccccatgggatcacaggagtgttcagggaagtggtgcttcctgggtctgggtgggctggaggggcatcctcccttccccaagaggagacccccaggagccccctaagtccatccccagcagtggtgcccctgccctgtccttgcagcctgggagacccttgggaggggcgggcgctgggtggctgggcggcttctgctggtctcaccccactggcctcctgtttgtcatcctcagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaagtatgccaggggcggcgcgggccgggtgggggctcagggctggcctacagccaccctgtgaccttgagcaggtctcaacccttgcagccccggcatccttgtgtttaaatggggagagtattgcacctgcttcctagggctgtgagacatcaagtgcgctcatgccaggcagtgcatggctgtatgcactgagtgtcccctgcacgcagggcacagggtgcaggtggaacattctccacgatgtcgccgtgaccagcgttccttccagccactgtcctctgagctctgtcctgcccttgagcaaagcccctgccccctgaggtatcctgtctccgggacgctagtcccaggagagggcacactcagacaggcttcaggctgccctgctggaaggtccctggggttaagcgttcttggccacagcattgctcatgcagagggttaggtaggggtgaggctagccgtgacagtattagcatttatggacgctaccaccccctccccttttccttaaacacatagtgcttttggtcacatgctgctttggaggaggcctcacttggcggatgtatttttctgccttagagagaggctgaactgggtttgactgttggcccagccctctcttgctgcgtgcccttagacgattcactcaacgtctctgatccatggcatgtacaactataagatgggcatgcccttctcctctcgggctgttatgaaggtcaaggaagcaagggctgttacccaagggtgctcccttctctccccctcttcacacccccaggtgctctgggccctctaggaactgggtttctctcaagggctgttacccaagggtgctcccttctctccccctcttcacaccactgggtgctctgggcccactaggagctgggattctcttaagagggaaactcttggataaaggaaatggtttgattgatatcggacaagtctgttcattagtatccatttattaagcacctaccatgtgccaggaaatgctttggcgtacaaaggaaaataagggccagtcctgctagaaatggccttgaaaccccagggagggatgtcggcccattgtgggtgctgcagattccttgaaggtgatgcaagagccagaaagaaggatgatgtggggggctgaggcagggagtcggggttgggggagtgtgggggagaaggggagaccgagcacctcttccactatctccctgtgtggtttttggtgaaccatcctgcctctgggtgtcttgcctccagcttctgacgttggaagttcatccactgagagctctgtgtttatggctctgagatactgagtccttcttctctcccagacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacggtacgtgtggctcaggcttggcaagcaggttggcagaatcttaaagagatgttgattggaaatgacacttgtgctatgccaaatggaagggaggcatttgcgttgagcgagggtagcgtgcagcgggtggccaatgggagaggctcacagaggctaagagcacctgccgcattttgggggaggcagcagccaccacatctgttctgtactgtactgagtggtggtgattcaagccaggcatggaaaaggctagaacagggctttcccactgcagcacccttgacatctgggtggttctctgttgtagggctctcttgtgccttgtaggatgtttaacagcgtccccagcctctacccactggaggccagtagctaccaagctgtgacaaccagtgttgcctgctgacattgccaaacatccgctttgaggcaaagtcacttccagttgagaactactggcctaaaatgtgtaaagatccttgatttttaaagatacattctaaaaccaagttgcttaattcaggacaaacatgctttctcttagcctcttattcggtcccactctggtccatccaagggtctggaatgttctagccccatgtggatacagaagaagcaaaacctcagccctccctacagcatgtctgtattcacattgggaaatggttcacatatagaagagcgaatgcctgagcaatggcgtggtgcctctggggcgaaagctgactccattgactccatcggctttttggctgttgcctcctgtgtgtctttcccgtcttgatcacctggagatatgtaattttggaagcagagctagcaaataattcctcttataagcagagctagcaaataattctacttataagtagcataacgtcttgcctgccagaaggagaggtctggcagggggagaaagtgagaatgtgggacttgttgggatgcagggtcctctgggcagggtggccagggtgccaggcccagcagcctgcatgtgggaaggccaggtggagacataggtgatacccgcctggctcactgtgttttctcttcttgaaacagacctagtgaaaccagtgtgtctgcccaacccaggcatgatgctgcagccagaacagctctgctggatttccgggtggggggccaccgaggagaaaggtgaggctgctcctgggcacacaggactgcagggcccacagatggagcattgggttcggaagtgggaggtccaggttttaatcccagttctactactcaatgactggatgactttggttgattcccccagtccttgtgcctcagtttctccatctgctaagtgggagaaatcctgcccagcctacctaatacactgtgttcttatcgtgatcacacagagcagcatgtggaatggcttttgaagtatctgggccatacgagtttagaggtgcaggatctcctgtgttgcactcattgtgagtttagagctgccctggagatcccaccaaggcctgcgtggctgagtgacagggggcttggtgaggacgggcatcctggacccatggtggccacatctaagcctgtcctctgccctgataaccacagagagaggctctctccacccacttcctttgcaatctgcatttctctctgacagtctttcaaatgaagggagcctggctgcttcatttttatggagggttggaagtgcttagtggcaggcacaaaggttcattttacatattgtttatatccttctcaaaagcgtctaggccatacagacaacaaatcctttcaaacaaggggaaaagtacaaaggttgggtgatttctggggagcgtcagggaaggtagtggggggcatcctggctcctcatcagcagaaacttactacagtagagccacaggctgggcaaaagacctcatggaatccaagatgaagggaatatcgacaaatatttgtgcgcacctgcacctagtacaggctgggtgctactcaggtgctgggaatgcagaagtgaacagagtaagacaaatgtctctgctgtcaggagctttacctctcttctggatgtcggtggtggggacggggcaggtgtggtcagacagatgggagacaaacaactgagcgaggtacttccaaacatctgagggtggggatcacaaggtcccggctattttgaaggggtggtcaggaaaggcttctcggaagaggtggcatttgagctgagactcaaatggcaaaaatgtgtacacatcaaaaaggctagtgcatgtatcttcaggtgtggtcaaggggccaaggaggtgggctggggccagattgcataggtccttgtggattatggtgaagacaccagcttctcatctgcttgaggtggggagatcgtgagccggggagtgccatgatctggcagctgcgtggggagtggggatgaatggatggagacgaggatgatggtgacaagtccattgctgtggttccttgagacaggaagccagctcatagcagagtgcgggcgtggatgtgaagagatgagggtacactagggctagagccaccagacttactgatgggttgcatgtctgtgggagagagagtgagaagtcagggacgatggctttccactctgtggctgaagccccagggtggcgggtggtgccatttttcaagccaggaaatattggttggtgagaatttggggtgggagaaggtgtgacggagggttctggttttgcacactaagcccacggtgcccagaagatgcccgaggggaggcagcaaagcgagagtgggaaatgcagaggtggcaagtgcaggccgtgtcttgagaagctctaatgtgcaggggagccgagaagcaggcggcctagggagggtcacgtgtgctccagaagagtgtgtgcatgccagaggggaaacaggcgcctgtgtgtcctgggtggggttcagtgaggagtgggaaattggttcagcagaaccaagccgttgggtgaataagagggggattccatggcactgatagagccctatagtttcagagctgggaatttctttccctgaagctgaactccagagctgcattcagcacaggcaccgccagttgtaaggagaatccaggtttcccaggagaggggttggtgctgggatgagctgaccggggcagggctggaaaatagggctgtgaccatctgtgtagtgcgtgtggaggtctcagggagggaagtgtgctctccctgcgagagctgcaggcaacactgggagctcaacaagtctccctgtccttagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccaggtaattcaacatttttattctacctttggtccttaccagatcctactgaaccccccatgagagagagggcattcttggggtcagcagagcctcctcagtgacacggagccagctcggggcagtcatgggaagtgacggccacaaacagtgcgaacgcttctggtggcagaaggaagtacagtcaacaaatcacacacaccctctgaaaaaccggtatttggtaaaagtgccagtggaacagaaacaagtatttagactattttaaattatgaacggcaatttatttagtaacttttagcttgaacagattaaaattcaggatgggggctatctctttgggggttacatctctgttaccatcaccccttgatggtggagattcgaagcccacacagtcactcgtaactcacactgcgacccccgccccccaactcctctaggcctggtcagtggtgtgcggcagattgtgacttgattttctgctctctgtaccttgctgtgtcccacagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggtaactatcctgtcctccttctgactgtgttctccgattcctcgagccaaagccagacatctgttaggcgtggttctgctgctggaagctgactggtgaccactggtcagcatgaagcaaactctgcttcctccagccacagccccatccccccagtgtccacccattgcccattgcctctcactggcttcacttgcatatttcccctggtgtttggatgaaaagcgctggggctcagcttgtgtgaaattccttggtgctctgccaaccacacttcgttctggctcagctgactcagctgttccacccaggccacctcacatcaaactttttttttttttttttgagatggagtctcactgtgtcgcccaggctggagtgcagtggcacaatctcgactcactgcaacctttgcctcctgggttcaagtgattctcctgcctcagcctcccaagtagctgggactacaggcatgcgccaccacgcccagctactttttgtatttttagtagagatggggtttctccatgttggccaggctggtctcgaagccctgacctcaggtgattcacccacctcagcctcccacagtgctgggattacaagtgtgaaccacggtgcccggcctcacatgaaacttttgatttatagagagcagagggaagagccggctgtgcccatccttttctggggccatcgagtggctcctgggcagcccccaaggttaggaagggcaggagcagccagggttctctgatgccccagactcaagcacgagggaaggtctcaggggttccatgtgagcctcatggatgtctctgcttagcagagccctggctttgggcattgtccagatagggggtgagaaccagatcttctcatctccaggacctcagacgtatagttttctcagatttctgtgctttctggggctgggctactagtggaagaaagcagtctattctgtcttctcccaaatctcccagatgcccagtctgttgaaggaggagcagaaccagggggcctttcccgctgaggcccgacctgtgtctccttcaaatgacacgcgggactcagggccttcccatgaccatggggcccagggggcgtcacctggcccagggcccagtgctagaaacagatgaccccaggaggaggaggcagggcaggagggaagctggcagggctgggatggtcagccaggctgaggggcggactcgcaccaggatggagctaggaaatgatccaggtgtgtttggcggctgcaggtgggtccgcatggctgtgcagggagggaagggctgcgtggcaggagagcagccgggggaggcccagactctgctgaagagatgcctgttgtgccggcctccacatccgctgcccgctccttccggagctcctgccccgccatgctcagcctgactctgaccaacacgttggagagaagaatgatccctttgtgctattaagcttgcttatttggtttctaagtgcttcatgcgaacctagaggaaaaaattattttccacctttgtttgtcttaagaaaataacacacttttttttttcctatttgaacaggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaactactgcatctttgaagttctgcctggtgagtaggaccagcctccatttccttataagggggtgatgttgaggctgctggtcagaggaccaaaggtgaggcaaggccagacttggtgctcctgtggttggtgccctcagttcctgcagcctgtcctgttggagaggtccctcaaatgactccttcttattattctattagtctgtttccatgctcctaataaagacatacccaagactgcaatttacaaaagaaagaagtttattggatttacaattccacatggctggggaggcctcacaatcatggcagaaagcaaggaagagcaaatcacatcttacatggatggcagcaggcagggagagagtttgtgcacagaaactcccattttttaaaccatcagatcttgtgagacccattcactatcacaaaaacagcacaggaaagacccacccccatgattcaattatctcctatcaggtccctcccacaacacatgggaattatgggagctacaagatgagatttgggtggggacacagagccaaaccatatcattctgcccctgcaccccccaaatctcgtatctttacatttcaaaaccaatcaggtcttcccaacagtcccccaaaatcttaactcatttcagcattaagtcaaaagtctacagtccaaagtctcatccaacacaaggcaagttgcttccacctatgagcctgtgaaatcaagagcaagttaattacttcctagatacaacgggggtacaggcattgggtgaatacagccattccaaatgggagaaattggccaaaataaaggggctgcaggccccttaaaagtccaaaatccagcagggcagtcaaatcttaaagcaccaaaatgacctcctttgactccatctgtcatatccaggtcacactgatgcaagaggtgggttcccatggtctggggcagctccacctctgtgacttcgcaaggtatagcttccttcctggctgcatcatgggctggtgttgagggtctgcagcttttccaggcacatggtgcaagctgttggtggatctaccattctggggtctggagaatggtggccctcttctcacagctccactaggcagtgccccagtagggactgtgtaggggctccaaccccacatttcccttctgcactgccctagcagaggttctccataagggccccaccctgcagcaaacttctgcctgggcatctatgcgtttccatacatcttctgaaatctaggcagaagttcccaaatctcaatccttgacttctgtgcactggcaggctcaacatcacatggaagctgccaaggcttaaggcttgcaacctctgaagccacagcccaagctctattttggcccctttcagccatggctggagtggctaggatgcagggcatcaagtctctaggctgcatacagcacaaggaccctgggcctggcccatgaaaccactttttcttcctaggcctctgggcctgttatgggaggggcttctgtgaaaacctctgacatgccctggagacactttctccattgtcttggggattaacattcagctcctcattacttatgcaaatttctgcagctgacttgaatttctactcagaaaatgggattttcttttctattgcattgtcaggctgcaacttttccaaacttttatgctgtttcccttttgaaactgaatgcctttaacagcacccaagtctctacttgaatgatttgctgcttagaaatttcttacaccaaataccctaaatcatctctctcaagttcaaagttccacaaatctctaaggcaggagcaaaatgccaccagtctctttgctaaaacataacaagagtaacctttgctccggttaccaacaagttcctcatctcttatctgagagcacctcagcctgaatttcattgtccatatcattatcagcatttttgtcaaagccattcaaaaggctgggcgtggtggctcacccct

SEQ ID NO: 2 - DNA, 24 bp, synthetic construct
  misc_feature 1..24: note = Synthetic nucleic acid
  source 1..24: mol_type = other DNA
cagagttggagcacttgctgccca

SEQ ID NO: 3 - DNA, 23 bp, synthetic construct
  misc_feature 1..23: note = Synthetic nucleic acid
  source 1..23: mol_type = other DNA
tcctgcttagctcgcgcctactc

SEQ ID NO: 4 - DNA, 25 bp, synthetic construct
  misc_feature 1..25: note = Synthetic nucleic acid
  source 1..25: mol_type = other DNA
tcaatatgacctgccgcgctccagg

SEQ ID NO: 5 - DNA, 25 bp, synthetic construct
  misc_feature 1..25: note = Synthetic nucleic acid
  source 1..25: mol_type = other DNA
aatgttcaatatgacctgccgcgct

SEQ ID NO: 6 - DNA, 25 bp, synthetic construct
  misc_feature 1..25: note = Synthetic nucleic acid
  source 1..25: mol_type = other DNA
ccaggttcccctccccagcccggac

SEQ ID NO: 7 - DNA, 25 bp, synthetic construct
  misc_feature 1..25: note = Synthetic nucleic acid
  source 1..25: mol_type = other DNA
accctgagttcaaagccatcttgct

SEQ ID NO: 8 - DNA, 25 bp, synthetic construct
  misc_feature 1..25: note = Synthetic nucleic acid
  source 1..25: mol_type = other DNA
gtggtgaccctgagttcaaagccat

SEQ ID NO: 9 - DNA, 25 bp, synthetic construct
  misc_feature 1..25: note = Synthetic nucleic acid
  source 1..25: mol_type = other DNA
ggtaataattaaccacttactgagt

SEQ ID NO: 10 - DNA, 25 bp, synthetic construct
  misc_feature 1..25: note = Synthetic nucleic acid
  source 1..25: mol_type = other DNA
gtggtgacccctaaacagttgaaaa

SEQ ID NO: 11 - DNA, 25 bp, synthetic construct
  misc_feature 1..25: note = Synthetic nucleic acid
  source 1..25: mol_type = other DNA
acaaagagaatcctacttgaggtgc

SEQ ID NO: 12 - DNA, 25 bp, synthetic construct
  misc_feature 1..25: note = Synthetic nucleic acid
  source 1..25: mol_type = other DNA
ttcttagtctctggaagaaggagga

SEQ ID NO: 13 - DNA, 25 bp, synthetic construct
  misc_feature 1..25: note = Synthetic nucleic acid
  source 1..25: mol_type = other DNA
gatcgaggctccctgcacttactga

SEQ ID NO: 14 - DNA, 25 bp, synthetic construct
  misc_feature 1..25: note = Synthetic nucleic acid
  source 1..25: mol_type = other DNA
ttgctgcccacttgcagagaaaaca

SEQ ID NO: 15 - DNA, 25 bp, synthetic construct
  misc_feature 1..25: note = Synthetic nucleic acid
  source 1..25: mol_type = other DNA
ggtcaaggctgactcaccacaccga

SEQ ID NO: 16 - DNA, 25 bp, synthetic construct
  misc_feature 1..25: note = Synthetic nucleic acid
  source 1..25: mol_type = other DNA
actgcacgagagggaggattatcca

SEQ ID NO: 17 - DNA, 25 bp, synthetic construct
  misc_feature 1..25: note = Synthetic nucleic acid
  source 1..25: mol_type = other DNA
cgggtgctgccccatactcacttat

SEQ ID NO: 18 - DNA, 25 bp, synthetic construct
  misc_feature 1..25: note = Synthetic nucleic acid
  source 1..25: mol_type = other DNA
gaagaaattgctgcatacctgtggt

SEQ ID NO: 19 - DNA, 25 bp, synthetic construct
  misc_feature 1..25: note = Synthetic nucleic acid
  source 1..25: mol_type = other DNA
aggcatcactgcaaaaagaacaggg

SEQ ID NO: 20 - DNA, 25 bp, synthetic construct
  misc_feature 1..25: note = Synthetic nucleic acid
  source 1..25: mol_type = other DNA
gggactccagatgaacttacctata

SEQ ID NO: 21 - DNA, 25 bp, synthetic construct
  misc_feature 1..25: note = Synthetic nucleic acid
  source 1..25: mol_type = other DNA
accccgcaggctgaggatgacaaac

SEQ ID NO: 22 - DNA, 25 bp, synthetic construct
  misc_feature 1..25: note = Synthetic nucleic acid
  source 1..25: mol_type = other DNA
ccgcccctggcatacttttccacgc

SEQ ID NO: 23 - DNA, 25 bp, synthetic construct
  misc_feature 1..25: note = Synthetic nucleic acid
  source 1..25: mol_type = other DNA
ctgggagagaagaaggactcagtat

SEQ ID NO: 24 - DNA, 25 bp, synthetic construct
  misc_feature 1..25: note = Synthetic nucleic acid
  source 1..25: mol_type = other DNA
ccaagcctgagccacacgtaccgtt

SEQ ID NO: 25 - DNA, 25 bp, synthetic construct
  misc_feature 1..25: note = Synthetic nucleic acid
  source 1..25: mol_type = other DNA
tcactaggtctgtttcaagaagaga

SEQ ID NO: 26 - DNA, 25 bp, synthetic construct
  misc_feature 1..25: note = Synthetic nucleic acid
  source 1..25: mol_type = other DNA
tgcccaggagcagcctcacctttct

SEQ ID NO: 27 - DNA, 25 bp, synthetic construct
  misc_feature 1..25: note = Synthetic nucleic acid
  source 1..25: mol_type = other DNA
ctaaggacagggagacttgttgagc

SEQ ID NO: 28 - DNA, 25 bp, synthetic construct
  misc_feature 1..25: note = Synthetic nucleic acid
  source 1..25: mol_type = other DNA
actgtcaccctgtgggacacagcaa

SEQ ID NO: 29 - DNA, 25 bp, synthetic construct
  misc_feature 1..25: note = Synthetic nucleic acid
  source 1..25: mol_type = other DNA
ggacaggatagttaccctcatttgt

SEQ ID NO: 30 - DNA, 25 bp, synthetic construct
  misc_feature 1..25: note = Synthetic nucleic acid
  source 1..25: mol_type = other DNA
aataattagccacttactgagttca

SEQ ID NO: 31 - DNA, 48 bp, synthetic construct
  misc_feature 1..48: note = Synthetic nucleic acid
  source 1..48: mol_type = other DNA
gagtaggcgcgagctaagcaggaggcggaggcggaggcggagggcgag

SEQ ID NO: 32 - DNA, 57 bp, synthetic construct
  misc_feature 1..57: note = Synthetic nucleic acid
  source 1..57: mol_type = other DNA
gggcgaggggcggggagcgccgcctggagcgcggcaggtcatattgaacattccaga

SEQ ID NO: 33 - DNA, 57 bp, synthetic construct
  misc_feature 1..57: note = Synthetic nucleic acid
  source 1..57: mol_type = other DNA
ccgcctggagcgcggcaggtcatattgaacattccagatacctatcattactcgatg

SEQ ID NO: 34 - DNA, 56 bp, synthetic construct
  misc_feature 1..56: note = Synthetic nucleic acid
  source 1..56: mol_type = other DNA
accagggtcccggctcggggtccgggctggggaggggaacctgggcgcctgggacc

SEQ ID NO: 35 - DNA, 52 bp, synthetic construct
  misc_feature 1..52: note = Synthetic nucleic acid
  source 1..52: mol_type = other DNA
ttactcgatgctgttgataacagcaagatggctttgaactcagggtcaccac

SEQ ID NO: 36 - DNA, 46 bp, synthetic construct
  misc_feature 1..46: note = Synthetic nucleic acid
  source 1..46: mol_type = other DNA
gatgctgttgataacagcaagatggctttgaactcagggtcaccac

SEQ ID NO: 37 - DNA, 36 bp, synthetic construct
  misc_feature 1..36: note = Synthetic nucleic acid
  source 1..36: mol_type = other DNA
ctttgaactcagtaagtggttaattattaccttcct

SEQ ID NO: 38 - DNA, 39 bp, synthetic construct
  misc_feature 1..39: note = Synthetic nucleic acid
  source 1..39: mol_type = other DNA
attcatcttcttttcaactgtttaggggtcaccaccagc

SEQ ID NO: 39 - DNA, 39 bp, synthetic construct
  misc_feature 1..39: note = Synthetic nucleic acid
  source 1..39: mol_type = other DNA
agtgtgcacctcaagtaggattctctttgtgtttatttc

SEQ ID NO: 40 - DNA, 37 bp, synthetic construct
  misc_feature 1..37: note = Synthetic nucleic acid
  source 1..37: mol_type = other DNA
cgttgcactctcctccttcttccagagactaagaaag

SEQ ID NO: 41 - DNA, 37 bp, synthetic construct
  misc_feature 1..37: note = Synthetic nucleic acid
  source 1..37: mol_type = other DNA
tctggaagttcagtaagtgcagggagcctcgatccca

SEQ ID NO: 42 - DNA, 37 bp, synthetic construct
  misc_feature 1..37: note = Synthetic nucleic acid
  source 1..37: mol_type = other DNA
acctctcttctgttttctctgcaagtgggcagcaagt

SEQ ID NO: 43 - DNA, 36 bp, synthetic construct
  misc_feature 1..36: note = Synthetic nucleic acid
  source 1..36: mol_type = other DNA
gaatcggtgtggtgagtcagccttgaccttgggaag

SEQ ID NO: 44 - DNA, 36 bp, synthetic construct
  misc_feature 1..36: note = Synthetic nucleic acid
  source 1..36: mol_type = other DNA
atggataatcctccctctcgtgcagttcgcctctac

SEQ ID NO: 45 - DNA, 37 bp, synthetic construct
  misc_feature 1..37: note = Synthetic nucleic acid
  source 1..37: mol_type = other DNA
catgggctataagtgagtatggggcagcacccgccga

SEQ ID NO: 46 - DNA, 37 bp, synthetic construct
  misc_feature 1..37: note = Synthetic nucleic acid
  source 1..37: mol_type = other DNA
actgtaccacaggtatgcagcaatttcttcttgaaaa

SEQ ID NO: 47 - DNA, 35 bp, synthetic construct
  misc_feature 1..35: note = Synthetic nucleic acid
  source 1..35: mol_type = other DNA
agaatttcccctgttctttttgcagtgatgcctgt

SEQ ID NO: 48 - DNA, 38 bp, synthetic construct
  misc_feature 1..38: note = Synthetic nucleic acid
  source 1..38: mol_type = other DNA
ttacgctgtataggtaagttcatctggagtcccccttt

SEQ ID NO: 49 - DNA, 37 bp, synthetic construct
  misc_feature 1..37: note = Synthetic nucleic acid
  source 1..37: mol_type = other DNA
ctggcctcctgtttgtcatcctcagcctgcggggtca

SEQ ID NO: 50 - DNA, 36 bp, synthetic construct
  misc_feature 1..36: note = Synthetic nucleic acid
  source 1..36: mol_type = other DNA
tgcgtggaaaagtatgccaggggcggcgcgggccgg

SEQ ID NO: 51 - DNA, 35 bp, synthetic construct
  misc_feature 1..35: note = Synthetic nucleic acid
  source 1..35: mol_type = other DNA
atactgagtccttcttctctcccagacctcttaac

SEQ ID NO: 52 - DNA, 37 bp, synthetic construct
  misc_feature 1..37: note = Synthetic nucleic acid
  source 1..37: mol_type = other DNA
tgactttcaacggtacgtgtggctcaggcttggcaag

SEQ ID NO: 53 - DNA, 36 bp, synthetic construct
  misc_feature 1..36: note = Synthetic nucleic acid
  source 1..36: mol_type = other DNA
actgtgttttctcttcttgaaacagacctagtgaaa

SEQ ID NO: 54 - DNA, 35 bp, synthetic construct
  misc_feature 1..35: note = Synthetic nucleic acid
  source 1..35: mol_type = other DNA
gaggagaaaggtgaggctgctcctgggcacacagg

SEQ ID NO: 55 - DNA, 38 bp, synthetic construct
  misc_feature 1..38: note = Synthetic nucleic acid
  source 1..38: mol_type = other DNA
gctcaacaagtctccctgtccttagggaagacctcaga

SEQ ID NO: 56 - DNA, 36 bp, synthetic construct
  misc_feature 1..36: note = Synthetic nucleic acid
  source 1..36: mol_type = other DNA
ctctgtaccttgctgtgtcccacagggtgacagtgg

SEQ ID NO: 57 - DNA, 36 bp, synthetic construct
  misc_feature 1..36: note = Synthetic nucleic acid
  source 1..36: mol_type = other DNA
gacaaatgagggtaactatcctgtcctccttctgac

SEQ ID NO: 58 - DNA, 36 bp, synthetic construct; identical to SEQ ID NO: 37
  misc_feature 1..36: note = Synthetic nucleic acid
  source 1..36: mol_type = other DNA

SEQ ID NO: 59 - protein, 14 aa, synthetic construct
  REGION 1..14: note = Synthetic peptide
  MOD_RES 2: note = Xaa = 6-aminohexanoic acid
  MOD_RES 5: note = Xaa = 6-aminohexanoic acid
  MOD_RES 8: note = Xaa = 6-aminohexanoic acid
  MOD_RES 11: note = Xaa = 6-aminohexanoic acid
  MOD_RES 13: note = Xaa = 6-aminohexanoic acid
  MOD_RES 14: note = Xaa = beta-alanine
RXRRXRRXRRXRXX

SEQ ID NO: 60 - protein, 26 aa, synthetic construct
  REGION 1..26: note = Synthetic peptide
LHHLLHHLLHLLHHLLHLLHHLLHHL

SEQ ID NO: 61 - protein, 10 aa, synthetic construct
  REGION 1..10: note = cell penetrating peptide
  MOD_RES 10: note = Xaa = glycine or proline
RFFRFFRFFX

SEQ ID NO: 62 - protein, 11 aa, synthetic construct
  REGION 1..11: note = cell penetrating peptide
  MOD_RES 11: note = Xaa = glycine or proline
RTRTRFLRRTX

SEQ ID NO: 63 - protein, 11 aa, synthetic construct
  REGION 1..11: note = cell penetrating peptide
  MOD_RES 11: note = Xaa = glycine or proline
RFFRFFRFFRX

SEQ ID NO: 64 - protein, 11 aa, synthetic construct
  REGION 1..11: note = cell penetrating peptide
  MOD_RES 11: note = Xaa = glycine or proline
KTRTKFLKKTX

SEQ ID NO: 65 - protein, 10 aa, synthetic construct
  REGION 1..10: note = cell penetrating peptide
  MOD_RES 10: note = Xaa = glycine or proline
KFFKFFKFFX

SEQ ID NO: 66 - protein, 11 aa, synthetic construct
  REGION 1..11: note = cell penetrating peptide
  MOD_RES 11: note = Xaa = glycine or proline
KFFKFFKFFKX

SEQ ID NO: 67 - protein, 7 aa, synthetic construct
  REGION 1..7: note = cell penetrating peptide
  MOD_RES 7: note = Xaa = glycine or proline
RFFRFFX

SEQ ID NO: 68 - protein, 8 aa, synthetic construct
  REGION 1..8: note = cell penetrating peptide
  MOD_RES 8: note = Xaa = glycine or proline
RFFRFFRX

SEQ ID NO: 69 - skipped

SEQ ID NO: 70 - protein, 13 aa, synthetic construct
  REGION 1..13: note = cell penetrating peptide
  MOD_RES 2: note = Xaa = 6-aminohexanoic acid
  MOD_RES 5: note = Xaa = 6-aminohexanoic acid
  MOD_RES 8: note = Xaa = 6-aminohexanoic acid
  MOD_RES 11: note = Xaa = 6-aminohexanoic acid
  MOD_RES 13: note = Xaa = glycine or proline
RXRRXRRXRRXRX

SEQ ID NO: 71 - protein, 12 aa, synthetic construct
  REGION 1..12: note = cell penetrating peptide
  MOD_RES 12: note = Xaa = glycine or proline
YGRKKRRQRRRX

SEQ ID NO: 72 - protein, 11 aa, synthetic construct
  REGION 1..11: note = cell penetrating peptide
  MOD_RES 11: note = Xaa = glycine or proline
GRKKRRQRRRX

SEQ ID NO: 73 - protein, 10 aa, synthetic construct
  REGION 1..10: note = cell penetrating peptide
  MOD_RES 10: note = Xaa = glycine or proline
RKKRRQRRRX

SEQ ID NO: 74 - protein, 19 aa, synthetic construct
  REGION 1..19: note = cell penetrating peptide
  MOD_RES 19: note = Xaa = glycine or proline
RQIKIWFQNRRMKWKKGGX

SEQ ID NO: 75 - protein, 28 aa, synthetic construct
  REGION 1..28: note = cell penetrating peptide
  MOD_RES 28: note = Xaa = glycine or proline
GWTLNSAGYLLGKINLKALAALAKKILX

SEQ ID NO: 76 - protein, 23 aa, synthetic construct
  REGION 1..23: note = cell penetrating peptide
  MOD_RES 23: note = Xaa = glycine or proline
YARVRRRGPRGYARVRRRGPRRX

SEQ ID NO: 77 - protein, 12 aa, synthetic construct
  REGION 1..12: note = cell penetrating peptide
  MOD_RES 12: note = Xaa = glycine or proline
YARVRRRGPRRX

SEQ ID NO: 78 - protein, 10 aa, synthetic construct
  REGION 1..10: note = cell penetrating peptide
  MOD_RES 10: note = Xaa = glycine or proline
AKAARQAARX

SEQ ID NO: 79 - protein, 35 aa, synthetic construct
  REGION 1..35: note = cell penetrating peptide
  MOD_RES 35: note = Xaa = glycine or proline
DAATATRGRSAASRPTERPRAPARSASRPRRPVEX

SEQ ID NO: 80 - protein, 22 aa, synthetic construct
  REGION 1..22: note = cell penetrating peptide
  MOD_RES 22: note = Xaa = glycine or proline
KETWWETWWTEWSQPKKKRKVX

SEQ ID NO: 81 - protein, 22 aa, synthetic construct
  REGION 1..22: note = cell penetrating peptide
  MOD_RES 22: note = Xaa = glycine or proline
KETWFETWFTEWSQPKKKRKVX

SEQ ID NO: 82 - protein, 17 aa, synthetic construct
  REGION 1..17: note = cell penetrating peptide
  MOD_RES 17: note = Xaa = glycine or proline
RQIKIWFQNRRMKWKKX

SEQ ID NO: 83 - protein, 25 aa, synthetic construct
  REGION 1..25: note = cell penetrating peptide
  MOD_RES 25: note = Xaa = glycine or proline
RRRRRRRQIKIWFQNRRMKWKKGGX

SEQ ID NO: 84 - protein, 11 aa, synthetic construct
  REGION 1..11: note = cell penetrating peptide
  MOD_RES 11: note = Xaa = glycine or proline
RRRQRRKKRCX

SEQ ID NO: 85 - protein, 13 aa, synthetic construct
  REGION 1..13: note = cell penetrating peptide
  MOD_RES 13: note = Xaa = glycine or proline
CYGRKKRRQRRRX

SEQ ID NO: 86 - protein, 13 aa, synthetic construct
  REGION 1..13: note = cell penetrating peptide
  MOD_RES 13: note = Xaa = glycine or proline
RRRRRRRRRFFCX

SEQ ID NO: 87 - protein, 13 aa, synthetic construct
  REGION 1..13: note = cell penetrating peptide
  MOD_RES 13: note = Xaa = glycine or proline
RRRRRRRRRCFFX

SEQ ID NO: 88 - protein, 13 aa, synthetic construct
  REGION 1..13: note = cell penetrating peptide
  MOD_RES 13: note = Xaa = glycine or proline
RRRRRRRRCFFRX

SEQ ID NO: 89 - protein, 13 aa, synthetic construct
  REGION 1..13: note = cell penetrating peptide
  MOD_RES 13: note = Xaa = glycine or proline
RRRRRRCFFRRRX

SEQ ID NO: 90 - protein, 13 aa, synthetic construct
  REGION 1..13: note = cell penetrating peptide
  MOD_RES 13: note = Xaa = glycine or proline
RRRRRFCFRRRRX

SEQ ID NO: 91 - protein, 12 aa, synthetic construct
  REGION 1..12: note = cell penetrating peptide
  MOD_RES 12: note = Xaa = glycine or proline
RRRRRFFRRRRX

SEQ ID NO: 92 - protein, 13 aa, synthetic construct
  REGION 1..13: note = cell penetrating peptide
  MOD_RES 13: note = Xaa = glycine or proline
RRRRCFFRRRRRX

SEQ ID NO: 93 - protein, 13 aa, synthetic construct
  REGION 1..13: note = cell penetrating peptide
  MOD_RES 13: note = Xaa = glycine or proline
RRCFFRRRRRRRX

SEQ ID NO: 94 - protein, 13 aa, synthetic construct
  REGION 1..13: note = cell penetrating peptide
  MOD_RES 13: note = Xaa = glycine or proline
CFFRRRRRRRRRX

SEQ ID NO: 95 - protein, 13 aa, synthetic construct
  REGION 1..13: note = cell penetrating peptide
  MOD_RES 13: note = Xaa = glycine or proline
CRRRRRRRRRFFX

SEQ ID NO: 96 - protein, 12 aa, synthetic construct
  REGION 1..12: note = cell penetrating peptide
  MOD_RES 12: note = Xaa = glycine or proline
FFRRRRRRRRRX

SEQ ID NO: 97 - protein, 15 aa, synthetic construct
  REGION 1..15: note = cell penetrating peptide
  MOD_RES 15: note = Xaa = glycine or proline
RRRRRFFCFFRRRRX

SEQ ID NO: 98 - protein, 12 aa, synthetic construct
  REGION 1..12: note = cell penetrating peptide
  MOD_RES 12: note = Xaa = glycine or proline
RRRRRRRRRIIX

SEQ ID NO: 99 - protein, 12 aa, synthetic construct
  REGION 1..12: note = cell penetrating peptide
  MOD_RES 12: note = Xaa = glycine or proline
RRRRRRRRFFFX

SEQ ID NO: 100 - protein, 14 aa, synthetic construct
  REGION 1..14: note = cell penetrating peptide
  MOD_RES 14: note = Xaa = glycine or proline
RRRRRRRRRFFFFX

SEQ ID NO: 101 - protein, 11 aa, synthetic construct
  REGION 1..11: note = cell penetrating peptide
  MOD_RES 11: note = Xaa = glycine or proline
RRRRRRRRFFX

SEQ ID NO: 102 - protein, 9 aa, synthetic construct
  REGION 1..9: note = cell penetrating peptide
  MOD_RES 9: note = Xaa = glycine or proline
RRRRRRFFX

SEQ ID NO: 103 - protein, 8 aa, synthetic construct
  REGION 1..8: note = cell penetrating peptide
  MOD_RES 8: note = Xaa = glycine or proline
RRRRRFFX

SEQ ID NO: 104 - protein, 12 aa, synthetic construct
  REGION 1..12: note = cell penetrating peptide
  MOD_RES 3: note = Xaa = 6-aminohexanoic acid
  MOD_RES 6: note = Xaa = 6-aminohexanoic acid
  MOD_RES 9: note = Xaa = 6-aminohexanoic acid
  MOD_RES 12: note = Xaa = glycine or proline
RRXRRXRRXRRX

SEQ ID NO: 105 - protein, 13 aa, synthetic construct
  REGION 1..13: note = cell penetrating peptide
  MOD_RES 2: note = Xaa = 6-aminohexanoic acid
  MOD_RES 5: note = Xaa = 6-aminohexanoic acid
  MOD_RES 8: note = Xaa = 6-aminohexanoic acid
  MOD_RES 11: note = Xaa = 6-aminohexanoic acid
  MOD_RES 13: note = Xaa = glycine or proline
RXRRXRRXRRXRX

SEQ ID NO: 106 - protein, 13 aa, synthetic construct
  REGION 1..13: note = cell penetrating peptide
  MOD_RES 1: note = Xaa = 6-aminohexanoic acid
  MOD_RES 4: note = Xaa = 6-aminohexanoic acid
  MOD_RES 7: note = Xaa = 6-aminohexanoic acid
  MOD_RES 10: note = Xaa = 6-aminohexanoic acid
  MOD_RES 13: note = Xaa = glycine or proline
XRRXRRXRRXRRX

SEQ ID NO: 107 - protein, 12 aa, synthetic construct
  REGION 1..12: note = cell penetrating peptide
  MOD_RES 2: note = Xaa = 6-aminohexanoic acid
  MOD_RES 4: note = Xaa = 6-aminohexanoic acid
  MOD_RES 6: note = Xaa = 6-aminohexanoic acid
  MOD_RES 8: note = Xaa = 6-aminohexanoic acid
  MOD_RES 10: note = Xaa = 6-aminohexanoic acid
  MOD_RES 12: note = Xaa = glycine or proline
RXRXRXRXRXRX

SEQ ID NO: 108 - protein, 10 aa, synthetic construct
  REGION 1..10: note = cell penetrating peptide
  MOD_RES 2: note = Xaa = 6-aminohexanoic acid
  MOD_RES 5: note = Xaa = 6-aminohexanoic acid
  MOD_RES 8: note = Xaa = 6-aminohexanoic acid
  MOD_RES 10: note = Xaa = glycine or proline
RXRRXRRXRX

SEQ ID NO: 109 - protein, 8 aa, synthetic construct
  REGION 1..8: note = cell penetrating peptide
  MOD_RES 2: note = Xaa = 6-aminohexanoic acid
  MOD_RES 5: note = Xaa = 6-aminohexanoic acid
  MOD_RES 8: note = Xaa = glycine or proline
RXRRXRRX

SEQ ID NO: 110 - protein, 7 aa, synthetic construct
  REGION 1..7: note = cell penetrating peptide
  MOD_RES 2: note = Xaa = 6-aminohexanoic acid
  MOD_RES 5: note = Xaa = 6-aminohexanoic acid
  MOD_RES 7: note = Xaa = glycine or proline
RXRRXRX

SEQ ID NO: 111 - protein, 12 aa, synthetic construct
  REGION 1..12: note = cell penetrating peptide
  MOD_RES 3: note = Xaa = 6-aminohexanoic acid
  MOD_RES 6: note = Xaa = 6-aminohexanoic acid
  MOD_RES 9: note = Xaa = 6-aminohexanoic acid
  MOD_RES 12: note = Xaa = glycine or proline
RKXRKXRKXRKX

SEQ ID NO: 112 - protein, 12 aa, synthetic construct
  REGION 1..12: note = cell penetrating peptide
  MOD_RES 3: note = Xaa = 6-aminohexanoic acid
  MOD_RES 6: note = Xaa = 6-aminohexanoic acid
  MOD_RES 9: note = Xaa = 6-aminohexanoic acid
  MOD_RES 12: note = Xaa = glycine or proline
RHXRHXRHXRHX

SEQ ID NO: 113 - protein, 13 aa, synthetic construct
  REGION 1..13: note = cell penetrating peptide
  MOD_RES 13: note = Xaa = glycine or proline
RRRRRRRRCFFRX

SEQ ID NO: 114 - protein, 12 aa, synthetic construct
  REGION 1..12: note = cell penetrating peptide
  MOD_RES 3: note = Xaa = 6-aminohexanoic acid
  MOD_RES 6: note = Xaa = 6-aminohexanoic acid
  MOD_RES 9: note = Xaa = 6-aminohexanoic acid
  MOD_RES 12: note = Xaa = glycine or proline
RRXRRXRRXRRX

SEQ ID NO: 115 - protein, 13 aa, synthetic construct
  REGION 1..13: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  MOD_RES 13: note = Xaa = glycine or proline
RXRRXRRXRRXRX

SEQ ID NO: 116 - protein, 13 aa, synthetic construct
  REGION 1..13: note = cell penetrating peptide
  MOD_RES 1..4: note = Xaa = 6-aminohexanoic acid
  SITE 7: note = misc_feature - Xaa can be any naturally occurring amino acid
  SITE 10: note = misc_feature - Xaa can be any naturally occurring amino acid
  MOD_RES 13: note = Xaa = glycine or proline
XRRXRRXRRXRRX

SEQ ID NO: 117 - protein, 12 aa, synthetic construct
  REGION 1..12: note = cell penetrating peptide
  MOD_RES 2..4: note = Xaa = 6-aminohexanoic acid
  SITE 6: note = misc_feature - Xaa can be any naturally occurring amino acid
  SITE 8: note = misc_feature - Xaa can be any naturally occurring amino acid
  SITE 10: note = misc_feature - Xaa can be any naturally occurring amino acid
  MOD_RES 12: note = Xaa = glycine or proline
RXRXRXRXRXRX

SEQ ID NO: 118 - protein, 14 aa, synthetic construct
  REGION 1..14: note = cell penetrating peptide
  MOD_RES 2..4: note = Xaa = 6-aminohexanoic acid
  SITE 6: note = misc_feature - Xaa can be any naturally occurring amino acid
  SITE 8: note = misc_feature - Xaa can be any naturally occurring amino acid
  SITE 10: note = misc_feature - Xaa can be any naturally occurring amino acid
  SITE 12: note = misc_feature - Xaa can be any naturally occurring amino acid
  MOD_RES 14: note = Xaa = glycine or proline
RXRXRXRXRXRXRX

SEQ ID NO: 119 - protein, 16 aa, synthetic construct
  REGION 1..16: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 14: note = misc_feature - Xaa can be any naturally occurring amino acid
  MOD_RES 16: note = Xaa = glycine or proline
RXRRXRRXRRXRRXRX

SEQ ID NO: 120 - protein, 13 aa, synthetic construct
  REGION 1..13: note = cell penetrating peptide
  MOD_RES 2..8: note = Xaa = 6-aminohexanoic acid
  MOD_RES 5..11: note = Xaa = beta-alanine
  MOD_RES 13: note = Xaa = glycine or proline
RXRRXRRXRRXRX

SEQ ID NO: 121 - protein, 13 aa, synthetic construct
  REGION 1..13: note = cell penetrating peptide
  MOD_RES 2..5: note = Xaa = 6-aminohexanoic acid
  SITE 8: note = misc_feature - Xaa can be any naturally occurring amino acid
  MOD_RES 11: note = Xaa = beta-alanine
  MOD_RES 13: note = Xaa = glycine or proline
RXRRXRRXRRXRX

SEQ ID NO: 122 - protein, 16 aa, synthetic construct
  REGION 1..16: note = cell penetrating peptide
  MOD_RES 2..4: note = Xaa = beta-alanine
  SITE 6: note = misc_feature - Xaa can be any naturally occurring amino acid
  SITE 8: note = misc_feature - Xaa can be any naturally occurring amino acid
  SITE 10: note = misc_feature - Xaa can be any naturally occurring amino acid
  MOD_RES 12: note = Xaa = 6-aminohexanoic acid
  SITE 14: note = misc_feature - Xaa can be any naturally occurring amino acid
  MOD_RES 16: note = Xaa = glycine or proline
RXRXRXRXRXRXRXRX

SEQ ID NO: 123 - protein, 16 aa, synthetic construct
  REGION 1..16: note = cell penetrating peptide
  MOD_RES 2..4: note = Xaa =beta-alanine
  SITE 6: note = misc_feature - Xaa can be any naturally occurring amino acid
  MOD_RES 8: note = Xaa = 6-aminohexanoic acid
  SITE 10: note = misc_feature - Xaa can be any naturally occurring amino acid
  SITE 12: note = misc_feature - Xaa can be any naturally occurring amino acid
  SITE 14: note = misc_feature - Xaa can be any naturally occurring amino acid
  MOD_RES 16: note = Xaa = glycine or proline
RXRXRXRXRXRXRXRX

SEQ ID NO: 124 - protein, 17 aa, synthetic construct
  REGION 1..17: note = cell penetrating peptide
  MOD_RES 1..9: note = Xaa = 6-aminohexanoic acid
  MOD_RES 3..5: note = Xaa = beta-alanine
  SITE 11: note = misc_feature - Xaa can be any naturally occurring amino acid
  SITE 13: note = misc_feature - Xaa can be any naturally occurring amino acid
  SITE 15: note = misc_feature - Xaa can be any naturally occurring amino acid
  MOD_RES 17: note = Xaa = glycine or proline
XRXRXRXRXRXRXRXRX

SEQ ID NO: 125 - protein, 16 aa, synthetic construct
  REGION 1..16: note = cell penetrating peptide
  MOD_RES 2..6: note = Xaa = beta-alanine
  MOD_RES 4..8: note = Xaa = 6-aminohexanoic acid
  SITE 10: note = misc_feature - Xaa can be any naturally occurring amino acid
  SITE 12: note = misc_feature - Xaa can be any naturally occurring amino acid
  SITE 14: note = misc_feature - Xaa can be any naturally occurring amino acid
  MOD_RES 16: note = Xaa = glycine or proline
RXRXRXRXRXRXRXRX

SEQ ID NO: 126 - protein, 16 aa, synthetic construct
  REGION 1..16: note = cell penetrating peptide
  MOD_RES 2..4: note = Xaa = beta-alanine
  SITE 6: note = misc_feature - Xaa can be any naturally occurring amino acid
  SITE 8: note = misc_feature - Xaa can be any naturally occurring amino acid
  MOD_RES 10..12: note = Xaa = 6-aminohexanoic acid
  SITE 14: note = misc_feature - Xaa can be any naturally occurring amino acid
  MOD_RES 16: note = Xaa = glycine or proline
RXRXRXRXRXRXRXRX

SEQ ID NO: 127 - protein, 16 aa, synthetic construct
  REGION 1..16: note = cell penetrating peptide
  MOD_RES 2..8: note = Xaa = 6-aminohexanoic acid
  MOD_RES 4..6: note = Xaa = beta-alanine
  SITE 10: note = misc_feature - Xaa can be any naturally occurring amino acid
  SITE 12: note = misc_feature - Xaa can be any naturally occurring amino acid
  SITE 14: note = misc_feature - Xaa can be any naturally occurring amino acid
  MOD_RES 16: note = Xaa = glycine or proline
RXRXRXRXRXRXRXRX

SEQ ID NO: 128 - protein, 16 aa, synthetic construct
  REGION 1..16: note = cell penetrating peptide
  MOD_RES 2..4: note = Xaa = beta-alanine
  SITE 6: note = misc_feature - Xaa can be any naturally occurring amino acid
  SITE 8: note = misc_feature - Xaa can be any naturally occurring amino acid
  SITE 10: note = misc_feature - Xaa can be any naturally occurring amino acid
  SITE 12: note = misc_feature - Xaa can be any naturally occurring amino acid
  SITE 14: note = misc_feature - Xaa can be any naturally occurring amino acid
  MOD_RES 16: note = Xaa = glycine or proline
RXRXRXRXRXRXRXRX

SEQ ID NO: 129 - protein, 5 aa, synthetic construct
  REGION 1..5: note = cell penetrating peptide
  MOD_RES 5: note = Xaa = glycine or proline
RRRRX

SEQ ID NO: 130 - protein, 6 aa, synthetic construct
  REGION 1..6: note = cell penetrating peptide
  MOD_RES 6: note = Xaa = glycine or proline
RRRRRX

SEQ ID NO: 131 - protein, 7 aa, synthetic construct
  REGION 1..7: note = cell penetrating peptide
  MOD_RES 7: note = Xaa = glycine or proline
RRRRRRX

SEQ ID NO: 132 - protein, 8 aa, synthetic construct
  REGION 1..8: note = cell penetrating peptide
  MOD_RES 8: note = Xaa = glycine or proline
RRRRRRRX

SEQ ID NO: 133 - protein, 9 aa, synthetic construct
  REGION 1..9: note = cell penetrating peptide
  MOD_RES 9: note = Xaa = glycine or proline
RRRRRRRRX

SEQ ID NO: 134 - protein, 11 aa, synthetic construct
  REGION 1..11: note = cell penetrating peptide
  MOD_RES 11: note = Xaa = glycine or proline
RRRRRGRRRRX

SEQ ID NO: 135 - protein, 12 aa, synthetic construct
  REGION 1..12: note = cell penetrating peptide
  MOD_RES 12: note = Xaa = glycine or proline
RRRRRFFRRRRX

SEQ ID NO: 136 - protein, 10 aa, synthetic construct
  REGION 1..10: note = cell penetrating peptide
  MOD_RES 10: note = Xaa = glycine or proline
RKKRRQRRRX

SEQ ID NO: 137 - protein, 10 aa, synthetic construct
  REGION 1..10: note = cell penetrating peptide
  MOD_RES 10: note = Xaa = glycine or proline
RRRQRRKKRX

SEQ ID NO: 138 - protein, 7 aa, synthetic construct
  REGION 1..7: note = cell penetrating peptide
  MOD_RES 2..5: note = Xaa = 6-aminohexanoic acid
  MOD_RES 7: note = Xaa = glycine or proline
RXRRXRX

SEQ ID NO: 139 - protein, 7 aa, synthetic construct
  REGION 1..7: note = cell penetrating peptide
  MOD_RES 2..5: note = Xaa = beta-alanine
  MOD_RES 7: note = Xaa = glycine or proline
RXRRXRX

SEQ ID NO: 140 - protein, 7 aa, synthetic construct
  REGION 1..7: note = cell penetrating peptide
  MOD_RES 2: note = Xaa = 6-aminohexanoic acid
  MOD_RES 5: note = Xaa = beta-alanine
  MOD_RES 7: note = Xaa = glycine or proline
RXRRXRX

SEQ ID NO: 141 - protein, 7 aa, synthetic construct
  REGION 1..7: note = cell penetrating peptide
  MOD_RES 2: note = Xaa = beta-alanine
  MOD_RES 5: note = Xaa = 6-aminohexanoic acid
  MOD_RES 7: note = Xaa = glycine or proline
RXRRXRX

SEQ ID NO: 142 - protein, 8 aa, synthetic construct
  REGION 1..8: note = cell penetrating peptide
  MOD_RES 2..6: note = Xaa = 6-aminohexanoic acid
  MOD_RES 4: note = Xaa = NH-(CHRe)n-C(O)-wherein n is 2 to 7 and each Re isindependently, at each occurrence, hydrogen or methyl
  MOD_RES 8: note = Xaa - glycine or proline
RXRXRXRX

SEQ ID NO: 143 - protein, 8 aa, synthetic construct
  REGION 1..8: note = cell penetrating peptide
  MOD_RES 2..6: note = Xaa = beta-alanine
  MOD_RES 4: note = Xaa = NH-(CHRe)n-C(O)-wherein n is 2 to 7 and each Re isindependently, at each occurrence, hydrogen or methyl
  MOD_RES 8: note = Xaa = glycine or proline
RXRXRXRX

SEQ ID NO: 144 - protein, 8 aa, synthetic construct
  REGION 1..8: note = cell penetrating peptide
  MOD_RES 2: note = Xaa = 6-aminohexanoic acid
  MOD_RES 4: note = Xaa = NH-(CHRe)n-C(O)-wherein n is 2 to 7 and each Re isindependently, at each occurrence, hydrogen or methyl
  MOD_RES 6: note = Xaa = beta-alanine
  MOD_RES 8: note = Xaa = glycine or proline
RXRXRXRX

SEQ ID NO: 145 - protein, 8 aa, synthetic construct
  REGION 1..8: note = cell penetrating peptide
  MOD_RES 2: note = Xaa = beta-alanine
  MOD_RES 4: note = Xaa = NH-(CHRe)n-C(O)-wherein n is 2 to 7 and each Re isindependently, at each occurrence, hydrogen or methyl
  MOD_RES 6: note = Xaa = 6-aminohexanoic acid
  MOD_RES 8: note = Xaa = glycine or proline
RXRXRXRX

SEQ ID NO: 146 - protein, 12 aa, synthetic construct
  REGION 1..12: note = cell penetrating peptide
  MOD_RES 2..10: note = Xaa = 6-aminohexanoic acid
  MOD_RES 12: note = Xaa = glycine or proline
RXRILFQYRXRX

SEQ ID NO: 147 - protein, 12 aa, synthetic construct
  REGION 1..12: note = cell penetrating peptide
  MOD_RES 2..10: note = Xaa = beta-alanine
  MOD_RES 12: note = Xaa = glycine or proline
RXRILFQYRXRX

SEQ ID NO: 148 - protein, 12 aa, synthetic construct
  REGION 1..12: note = cell penetrating peptide
  MOD_RES 2: note = Xaa = 6-aminohexanoic acid
  MOD_RES 10: note = Xaa = beta-alanine
  MOD_RES 12: note = Xaa = glycine or proline
RXRILFQYRXRX

SEQ ID NO: 149 - protein, 12 aa, synthetic construct
  REGION 1..12: note = cell penetrating peptide
  MOD_RES 2: note = Xaa = beta-alanine
  MOD_RES 10: note = Xaa = 6-aminohexanoic acid
  MOD_RES 12: note = Xaa = glycine or proline
RXRILFQYRXRX

SEQ ID NO: 150 - protein, 10 aa, synthetic construct
  REGION 1..10: note = cell penetrating peptide
  MOD_RES 2..5: note = Xaa - 6-aminohexanoic acid
  SITE 8: note = misc_feature - Xaa can be any naturally occurring amino acid
  MOD_RES 10: note = Xaa = glycine or proline
RXRRXRRXRX

SEQ ID NO: 151 - protein, 10 aa, synthetic construct
  REGION 1..10: note = cell penetrating peptide
  MOD_RES 2..5: note = Xaa = beta-alanine
  SITE 8: note = misc_feature - Xaa can be any naturally occurring amino acid
  MOD_RES 10: note = Xaa = glycine or proline
RXRRXRRXRX

SEQ ID NO: 152 - protein, 10 aa, synthetic construct
  REGION 1..10: note = cell penetrating peptide
  MOD_RES 2..8: note = Xaa = 6-aminohexanoic acid
  MOD_RES 5: note = Xaa = beta-alanine
  MOD_RES 10: note = Xaa = glycine or proline
RXRRXRRXRX

SEQ ID NO: 153 - protein, 10 aa, synthetic construct
  REGION 1..10: note = cell penetrating peptide
  MOD_RES 2: note = Xaa = 6-aminohexanoic acid
  MOD_RES 5..8: note = Xaa = beta-alanine
  MOD_RES 10: note = Xaa = glycine or proline
RXRRXRRXRX

SEQ ID NO: 154 - protein, 10 aa, synthetic construct
  REGION 1..10: note = cell penetrating peptide
  MOD_RES 2..5: note = Xaa = 6-aminohexanoic acid
  MOD_RES 8: note = Xaa = beta-alanine
  MOD_RES 10: note = Xaa = glycine or proline
RXRRXRRXRX

SEQ ID NO: 155 - protein, 10 aa, synthetic construct
  REGION 1..10: note = cell penetrating peptide
  MOD_RES 2..8: note = Xaa = beta-alanine
  MOD_RES 5: note = Xaa = 6-aminohexanoic acid
  MOD_RES 10: note = Xaa = glycine or proline
RXRRXRRXRX

SEQ ID NO: 156 - protein, 10 aa, synthetic construct
  REGION 1..10: note = cell penetrating peptide
  MOD_RES 2: note = Xaa = beta-alanine
  MOD_RES 5..8: note = Xaa = 6-aminohexanoic acid
  MOD_RES 10: note = Xaa = glycine or proline
RXRRXRRXRX

SEQ ID NO: 157 - protein, 10 aa, synthetic construct
  REGION 1..10: note = cell penetrating peptide
  MOD_RES 2..5: note = Xaa = beta-alanine
  MOD_RES 8: note = Xaa = 6-aminohexanoic acid
  MOD_RES 10: note = Xaa = glycine or proline
RXRRXRRXRX

SEQ ID NO: 158 - protein, 11 aa, synthetic construct
  REGION 1..11: note = cell penetrating peptide
  MOD_RES 2..6: note = Xaa = 6-aminohexanoic acid
  MOD_RES 4: note = Xaa = -NH-(CHRe)n-C(O)-wherein n is 2 to 7 and each Re isindependently, at each occurrence, hydrogen or methyl
  SITE 9: note = misc_feature - Xaa can be any naturally occurring amino acid
  MOD_RES 11: note = Xaa = glycine or proline
RXRXRXRRXRX

SEQ ID NO: 159 - protein, 11 aa, synthetic construct
  REGION 1..11: note = cell penetrating peptide
  MOD_RES 2..5: note = Xaa = 6-aminohexanoic acid
  MOD_RES 7: note = Xaa = -NH-(CHRe)n-C(O)-wherein n is 2 to 7 and each Re isindependently, at each occurrence, hydrogen or methyl
  SITE 9: note = misc_feature - Xaa can be any naturally occurring amino acid
  MOD_RES 11: note = Xaa = glycine or proline
RXRRXRXRXRX

SEQ ID NO: 160 - protein, 15 aa, synthetic construct
  REGION 1..15: note = cell penetrating peptide
  MOD_RES 2..10: note = Xaa = 6-aminohexanoic acid
  SITE 13: note = misc_feature - Xaa can be any naturally occurring amino acid
  MOD_RES 15: note = Xaa = glycine or proline
RXRILFQYRXRRXRX

SEQ ID NO: 161 - protein, 15 aa, synthetic construct
  REGION 1..15: note = cell penetrating peptide
  MOD_RES 2..5: note = Xaa = 6-aminohexanoic acid
  SITE 13: note = misc_feature - Xaa can be any naturally occurring amino acid
  MOD_RES 15: note = Xaa = glycine or proline
RXRRXRILFQYRXRX

SEQ ID NO: 162 - protein, 12 aa, synthetic construct
  REGION 1..12: note = cell penetrating peptide
  MOD_RES 2..6: note = Xaa = 6-aminohexanoic acid
  MOD_RES 4..8: note = Xaa = -NH-(CHRe)n-C(O)-wherein n is 2 to 7 and each Re isindependently, at each occurrence, hydrogen or methyl
  SITE 10: note = misc_feature - Xaa can be any naturally occurring amino acid
  MOD_RES 12: note = Xaa = glycine or proline
RXRXRXRXRXRX

SEQ ID NO: 163 - protein, 20 aa, synthetic construct
  REGION 1..20: note = cell penetrating peptide
  MOD_RES 2..10: note = Xaa = 6-aminohexanoic acid
  SITE 18: note = misc_feature - Xaa can be any naturally occurring amino acid
  MOD_RES 20: note = Xaa = glycine or proline
RXRILFQYRXRILFQYRXRX

SEQ ID NO: 164 - protein, 16 aa, synthetic construct
  REGION 1..16: note = cell penetrating peptide
  MOD_RES 2..10: note = Xaa = 6-aminohexanoic acid
  MOD_RES 12: note = Xaa = -NH-(CHRe)n-C(O)-wherein n is 2 to 7 and each Re isindependently, at each occurrence, hydrogen or methyl
  SITE 14: note = misc_feature - Xaa can be any naturally occurring amino acid
  MOD_RES 16: note = Xaa = glycine or proline
RXRILFQYRXRXRXRX

SEQ ID NO: 165 - protein, 16 aa, synthetic construct
  REGION 1..16: note = cell penetrating peptide
  MOD_RES 2..6: note = Xaa = 6-aminohexanoic acid
  MOD_RES 4: note = Xaa = -NH-(CHRe)n-C(O)-wherein n is 2 to 7 and each Re isindependently, at each occurrence, hydrogen or methyl
  SITE 14: note = misc_feature - Xaa can be any naturally occurring amino acid
  MOD_RES 16: note = Xaa = glycine or proline
RXRXRXRILFQYRXRX

SEQ ID NO: 166 - protein, 11 aa, synthetic construct
  REGION 1..11: note = cell penetrating peptide
  MOD_RES 2..6: note = Xaa = beta-alanine
  MOD_RES 4: note = Xaa = -NH-(CHRe)n-C(O)-wherein n is 2 to 7 and each Re isindependently, at each occurrence, hydrogen or methyl
  SITE 9: note = misc_feature - Xaa can be any naturally occurring amino acid
  MOD_RES 11: note = Xaa = glycine or proline
RXRXRXRRXRX

SEQ ID NO: 167 - protein, 11 aa, synthetic construct
  REGION 1..11: note = cell penetrating peptide
  MOD_RES 2..5: note = Xaa = beta-alanine
  MOD_RES 7: note = Xaa = -NH-(CHRe)n-C(O)-wherein n is 2 to 7 and each Re isindependently, at each occurrence, hydrogen or methyl
  SITE 9: note = misc_feature - Xaa can be any naturally occurring amino acid
  MOD_RES 11: note = Xaa = glycine or proline
RXRRXRXRXRX

SEQ ID NO: 168 - protein, 15 aa, synthetic construct
  REGION 1..15: note = cell penetrating peptide
  MOD_RES 2..10: note = Xaa = beta-alanine
  SITE 13: note = misc_feature - Xaa can be any naturally occurring amino acid
  MOD_RES 15: note = Xaa = glycine or proline
RXRILFQYRXRRXRX

SEQ ID NO: 169 - protein, 15 aa, synthetic construct
  REGION 1..15: note = cell penetrating peptide
  MOD_RES 2..5: note = Xaa = beta-alanine
  SITE 13: note = misc_feature - Xaa can be any naturally occurring amino acid
  MOD_RES 15: note = Xaa = glycine or proline
RXRRBRILFQYRXRX

SEQ ID NO: 170 - protein, 12 aa, synthetic construct
  REGION 1..12: note = cell penetrating peptide
  MOD_RES 2..6: note = Xaa = beta-alanine
  MOD_RES 8: note = Xaa = -NH-(CHRe)n-C(O)-wherein n is 2 to 7 and each Re isindependently, at each occurrence, hydrogen or methyl
  SITE 10: note = misc_feature - Xaa can be any naturally occurring amino acid
  MOD_RES 12: note = Xaa = glycine or proline
RXRYRXRXRXRX

SEQ ID NO: 171 - protein, 20 aa, synthetic construct
  REGION 1..20: note = cell penetrating peptide
  MOD_RES 2..10: note = Xaa = beta-alanine
  SITE 18: note = misc_feature - Xaa can be any naturally occurring amino acid
  MOD_RES 20: note = Xaa = glycine or proline
RXRILFQYRXRILFQYRXRX

SEQ ID NO: 172 - protein, 16 aa, synthetic construct
  REGION 1..16: note = cell penetrating peptide
  MOD_RES 2..6: note = Xaa = beta-alanine
  MOD_RES 4: note = Xaa = -NH-(CHRe)n-C(O)-wherein n is 2 to 7 and each Re isindependently, at each occurrence, hydrogen or methyl
  SITE 14: note = misc_feature - Xaa can be any naturally occurring amino acid
  MOD_RES 16: note = Xaa = glycine or alanine
RXRXRXRILFQYRXRX

SEQ ID NO: 173 - protein, 16 aa, synthetic construct
  REGION 1..16: note = cell penetrating peptide
  MOD_RES 2..10: note = Xaa = beta-alanine
  MOD_RES 12: note = Xaa = -NH-(CHRe)n-C(O)-wherein n is 2 to 7 and each Re isindependently, at each occurrence, hydrogen or methyl
  SITE 14: note = misc_feature - Xaa can be any naturally occurring amino acid
  MOD_RES 16: note = Xaa = glycine or proline
RXRILFQYRXRXRXRX

SEQ ID NO: 174 - protein, 11 aa, synthetic construct
  REGION 1..11: note = cell penetrating peptide
  MOD_RES 2..9: note = Xaa = 6-aminohexanoic acid
  MOD_RES 4: note = Xaa = -NH-(CHRe)n-C(O)-wherein n is 2 to 7 and each Re isindependently, at each occurrence, hydrogen or methyl
  MOD_RES 6: note = Xaa = beta-alanine
  MOD_RES 11: note = Xaa = glycine or proline
RXRXRXRRXRX

SEQ ID NO: 175 - protein, 11 aa, synthetic construct
  REGION 1..11: note = cell penetrating peptide
  MOD_RES 2..9: note = Xaa = 6-aminohexanoic acid
  MOD_RES 5: note = Xaa = beta-alanine
  MOD_RES 7: note = Xaa = -NH-(CHRe)n-C(O)-wherein n is 2 to 7 and each Re isindependently, at each occurrence, hydrogen or methyl
  MOD_RES 11: note = Xaa = glycine or proline
RXRRXRXRXRX

SEQ ID NO: 176 - protein, 15 aa, synthetic construct
  REGION 1..15: note = cell penetrating peptide
  MOD_RES 2..13: note = Xaa = 6-aminohexanoic acid
  MOD_RES 10: note = Xaa = beta-alanine
  MOD_RES 15: note = Xaa = glycine or proline
RXRILFQYRXRRXRX

SEQ ID NO: 177 - protein, 15 aa, synthetic construct
  REGION 1..15: note = cell penetrating peptide
  MOD_RES 2..13: note = Xaa = 6-aminohexanoic acid
  MOD_RES 5: note = Xaa = beta-alanine
  MOD_RES 15: note = Xaa = glycine or proline
RXRRXRILFQYRXRX

SEQ ID NO: 178 - protein, 12 aa, synthetic construct
  REGION 1..12: note = cell penetrating peptide
  MOD_RES 2..10: note = Xaa = 6-aminohexanoic acid
  MOD_RES 4..8: note = Xaa = -NH-(CHRe)n-C(O)-wherein n is 2 to 7 and each Re isindependently, at each occurrence, hydrogen or methyl
  MOD_RES 6: note = Xaa = beta-alanine
  MOD_RES 12: note = Xaa = glycine or proline
RXRXRXRXRXRX

SEQ ID NO: 179 - protein, 20 aa, synthetic construct
  REGION 1..20: note = cell penetrating peptide
  MOD_RES 2..18: note = Xaa = 6-aminohexanoic acid
  MOD_RES 10: note = Xaa = beta-alanine
  MOD_RES 20: note = Xaa = glycine or proline
RXRILFQYRXRILFQYRXRX

SEQ ID NO: 180 - protein, 16 aa, synthetic construct
  REGION 1..16: note = cell penetrating peptide
  MOD_RES 2..14: note = Xaa = 6-aminohexanoic acid
  MOD_RES 4: note = Xaa = -NH-(CHRe)n-C(O)-wherein n is 2 to 7 and each Re isindependently, at each occurrence, hydrogen or methyl
  MOD_RES 6: note = Xaa = beta-alanine
  MOD_RES 16: note = Xaa = glycine or proline
RXRXRXRILFQYRXRX

SEQ ID NO: 181 - protein, 16 aa, synthetic construct
  REGION 1..16: note = cell penetrating peptide
  MOD_RES 2..14: note = Xaa = 6-aminohexanoic acid
  MOD_RES 10: note = Xaa = beta-alanine
  MOD_RES 12: note = Xaa = -NH-(CHRe)n-C(O)-wherein n is 2 to 7 and each Re isindependently, at each occurrence, hydrogen or methyl
  MOD_RES 16: note = Xaa = glycine or proline
RXRILFQYRXRXRXRX

SEQ ID NO: 182 - protein, 11 aa, synthetic construct
  REGION 1..11: note = cell penetrating peptide
  MOD_RES 2: note = Xaa = 6-aminohexanoic acid
  MOD_RES 4: note = Xaa = -NH-(CHRe)n-C(O)-wherein n is 2 to 7 and each Re isindependently, at each occurrence, hydrogen or methyl
  MOD_RES 6..9: note = Xaa = beta-alanine
  MOD_RES 11: note = Xaa = glycine or proline
RXRXRXRRXRX

SEQ ID NO: 183 - protein, 11 aa, synthetic construct
  REGION 1..11: note = cell penetrating peptide
  MOD_RES 2: note = Xaa = 6-aminohexanoic acid
  MOD_RES 5..9: note = Xaa = beta-alanine
  MOD_RES 7: note = Xaa = -NH-(CHRe)n-C(O)-wherein n is 2 to 7 and each Re isindependently, at each occurrence, hydrogen or methyl
  MOD_RES 11: note = Xaa = glycine or proline
RXRRXRXRXRX

SEQ ID NO: 184 - protein, 15 aa, synthetic construct
  REGION 1..15: note = cell penetrating peptide
  MOD_RES 2: note = Xaa = 6-aminohexanoic acid
  MOD_RES 10..13: note = Xaa = beta-alanine
  MOD_RES 15: note = Xaa = glycine or proline
RXRILFQYRXRRXRX

SEQ ID NO: 185 - protein, 15 aa, synthetic construct
  REGION 1..15: note = cell penetrating peptide
  MOD_RES 2: note = Xaa = 6-aminohexanoic acid
  MOD_RES 5..13: note = Xaa = beta-alanine
  MOD_RES 15: note = Xaa = glycine or proline
RXRRXRILFQYRXRX

SEQ ID NO: 186 - protein, 12 aa, synthetic construct
  REGION 1..12: note = cell penetrating peptide
  MOD_RES 2: note = Xaa = 6-aminohexanoic acid
  MOD_RES 4..8: note = Xaa = -NH-(CHRe)n-C(O)-wherein n is 2 to 7 and each Re isindependently, at each occurrence, hydrogen or methyl
  MOD_RES 6..10: note = Xaa = beta-alanine
  MOD_RES 12: note = Xaa = glycine or proline
RXRXRXRXRXRX

SEQ ID NO: 187 - protein, 20 aa, synthetic construct
  REGION 1..20: note = cell penetrating peptide
  MOD_RES 2: note = Xaa = 6-aminohexanoic acid
  MOD_RES 10..18: note = Xaa = beta-alanine
  MOD_RES 20: note = Xaa = glycine or proline
RXRILFQYRXRILFQYRXRX

SEQ ID NO: 188 - protein, 16 aa, synthetic construct
  REGION 1..16: note = cell penetrating peptide
  MOD_RES 2: note = Xaa = 6-aminohexanoic acid
  MOD_RES 4: note = Xaa = -NH-(CHRe)n-C(O)-wherein n is 2 to 7 and each Re isindependently, at each occurrence, hydrogen or methyl
  MOD_RES 6..14: note = Xaa = beta-alanine
  MOD_RES 16: note = Xaa = glycine or proline
RXRXRXRILFQYRXRX

SEQ ID NO: 189 - protein, 16 aa, synthetic construct
  REGION 1..16: note = cell penetrating peptide
  MOD_RES 2: note = Xaa = 6-aminohexanoic acid
  MOD_RES 10..14: note = Xaa = beta-alanine
  MOD_RES 12: note = Xaa = -NH-(CHRe)n-C(O)-wherein n is 2 to 7 and each Re isindependently, at each occurrence, hydrogen or methyl
  MOD_RES 16: note = Xaa = glycine or proline
RXRILFQYRXRXRXRX

SEQ ID NO: 190 - protein, 11 aa, synthetic construct
  REGION 1..11: note = cell penetrating peptide
  MOD_RES 2..6: note = Xaa = 6-aminohexanoic acid
  MOD_RES 4: note = Xaa = -NH-(CHRe)n-C(O)-wherein n is 2 to 7 and each Re isindependently, at each occurrence, hydrogen or methyl
  MOD_RES 9: note = Xaa = beta-alanine
  MOD_RES 11: note = Xaa = glycine or proline
RXRXRXRRXRX

SEQ ID NO: 191 - protein, 11 aa, synthetic construct
  REGION 1..11: note = cell penetrating peptide
  MOD_RES 2..5: note = Xaa = 6-aminohexanoic acid
  MOD_RES 7: note = Xaa = -NH-(CHRe)n-C(O)-wherein n is 2 to 7 and each Re isindependently, at each occurrence, hydrogen or methyl
  MOD_RES 9: note = Xaa = beta-alanine
  MOD_RES 11: note = Xaa = glycine or proline
RXRRXRXRXRX

SEQ ID NO: 192 - protein, 15 aa, synthetic construct
  REGION 1..15: note = cell penetrating peptide
  MOD_RES 2..10: note = Xaa = 6-aminohexanoic acid
  MOD_RES 13: note = Xaa = beta-alanine
  MOD_RES 15: note = Xaa = glycine or alanine
RXRILFQYRXRRXRX

SEQ ID NO: 193 - protein, 15 aa, synthetic construct
  REGION 1..15: note = cell penetrating peptide
  MOD_RES 2..5: note = Xaa = 6-aminohexanoic acid
  MOD_RES 13: note = Xaa = beta-alanine
  MOD_RES 15: note = Xaa = glycine or proline
RXRRXRLLFQYRXRX

SEQ ID NO: 194 - protein, 12 aa, synthetic construct
  REGION 1..12: note = cell penetrating peptide
  MOD_RES 2..6: note = Xaa = 6-aminohexanoic acid
  MOD_RES 4..8: note = Xaa = -NH-(CHRe)n-C(O)-wherein n is 2 to 7 and each Re isindependently, at each occurrence, hydrogen or methyl
  MOD_RES 10: note = Xaa = beta-alanine
  MOD_RES 12: note = Xaa = glycine or proline
RXRXRXRXRXRX

SEQ ID NO: 195 - protein, 20 aa, synthetic construct
  REGION 1..20: note = cell penetrating peptide
  MOD_RES 2..10: note = Xaa = 6-aminohexanoic acid
  MOD_RES 18: note = Xaa = beta-alanine
  MOD_RES 20: note = Xaa = glycine or proline
RXRILFQYRXRILFQYRXRX

SEQ ID NO: 196 - protein, 16 aa, synthetic construct
  REGION 1..16: note = cell penetrating peptide
  MOD_RES 2: note = Xaa = 6-aminohexanoic acid
  MOD_RES 4: note = Xaa = -NH-(CHRe)n-C(O)-wherein n is 2 to 7 and each Re isindependently, at each occurrence, hydrogen or methyl
  MOD_RES 6: note = Xaa = 6-aminohexanoic acid
  MOD_RES 14: note = Xaa = beta-alanine
  MOD_RES 16: note = Xaa = glycine or proline
RXRXRXRILFQYRXRX

SEQ ID NO: 197 - protein, 16 aa, synthetic construct
  REGION 1..16: note = cell penetrating peptide
  MOD_RES 2..10: note = Xaa = 6-aminohexanoic acid
  MOD_RES 12: note = Xaa = -NH-(CHRe)n-C(O)-wherein n is 2 to 7 and each Re isindependently, at each occurrence, hydrogen or methyl
  MOD_RES 14: note = Xaa = beta-alanine
  MOD_RES 16: note = Xaa = glycine or proline
RXRILFQYRXRXRXRX

SEQ ID NO: 198 - protein, 11 aa, synthetic construct
  REGION 1..11: note = cell penetrating peptide
  MOD_RES 2..9: note = Xaa = beta-alanine
  MOD_RES 4: note = Xaa = -NH-(CHRe)n-C(O)-wherein n is 2 to 7 and each Re isindependently, at each occurrence, hydrogen or methyl
  MOD_RES 6: note = Xaa = 6-aminohexanoic acid
  MOD_RES 11: note = Xaa = glycine or proline
RXRXRXRRXRX

SEQ ID NO: 199 - protein, 11 aa, synthetic construct
  REGION 1..11: note = cell penetrating peptide
  MOD_RES 2..9: note = Xaa = beta-alanine
  MOD_RES 5: note = Xaa = 6-aminohexanoic acid
  MOD_RES 7: note = Xaa = -NH-(CHRe)n-C(O)-wherein n is 2 to 7 and each Re isindependently, at each occurrence, hydrogen or methyl
  MOD_RES 11: note = Xaa = glycine or proline
RXRRXRXRXRX

SEQ ID NO: 200 - protein, 15 aa, synthetic construct
  REGION 1..15: note = cell penetrating peptide
  MOD_RES 2..13: note = Xaa = beta-alanine
  MOD_RES 10: note = Xaa = 6-aminohexanoic acid
  MOD_RES 15: note = Xaa - glycine or proline
RXRILFQYRXRRXRX

SEQ ID NO: 201 - protein, 15 aa, synthetic construct
  REGION 1..15: note = cell penetrating peptide
  MOD_RES 2..13: note = Xaa = beta-alanine
  MOD_RES 5: note = Xaa = 6-aminohexanoic acid
  MOD_RES 15: note = Xaa = glycine or proline
RXRRXRILFQYRXRX

SEQ ID NO: 202 - protein, 12 aa, synthetic construct
  REGION 1..12: note = cell penetrating peptide
  MOD_RES 2..10: note = Xaa = beta-alanine
  MOD_RES 4..8: note = Xaa = -NH-(CHRe)n-C(O)-wherein n is 2 to 7 and each Re isindependently, at each occurrence, hydrogen or methyl
  MOD_RES 6: note = Xaa = 6-aminohexanoic acid
  MOD_RES 12: note = Xaa = glycine or proline
RXRXRXRXRXRX

SEQ ID NO: 203 - protein, 20 aa, synthetic construct
  REGION 1..20: note = cell penetrating peptide
  MOD_RES 2..18: note = Xaa = beta-alanine
  MOD_RES 10: note = Xaa = 6-aminohexanoic acid
  MOD_RES 20: note = Xaa = glycine or proline
RXRILFQYRXRILFQYRXRX

SEQ ID NO: 204 - protein, 16 aa, synthetic construct
  REGION 1..16: note = cell penetrating peptide
  MOD_RES 2..14: note = Xaa = beta-alanine
  MOD_RES 4: note = Xaa = -NH-(CHRe)n-C(O)-wherein n is 2 to 7 and each Re isindependently, at each occurrence, hydrogen or methyl
  MOD_RES 6: note = Xaa = 6-aminihexanoic acid
  MOD_RES 16: note = Xaa = glycine or proline
RXRXRXRILFQYRXRX

SEQ ID NO: 205 - protein, 16 aa, synthetic construct
  REGION 1..16: note = cell penetrating peptide
  MOD_RES 2..14: note = Xaa = beta-alanine
  MOD_RES 10: note = Xaa = 6-aminohexanoic acid
  MOD_RES 12: note = Xaa = -NH-(CHRe)n-C(O)-wherein n is 2 to 7 and each Re isindependently, at each occurrence, hydrogen or methyl
  MOD_RES 16: note = Xaa = glycine or proline
RXRILFQYRXRXRXRX

SEQ ID NO: 206 - protein, 11 aa, synthetic construct
  REGION 1..11: note = cell penetrating peptide
  MOD_RES 2: note = Xaa = beta-alanine
  MOD_RES 4: note = Xaa = -NH-(CHRe)n-C(O)-wherein n is 2 to 7 and each Re isindependently, at each occurrence, hydrogen or methyl
  MOD_RES 6..9: note = Xaa = 6-aminohexanoic acid
  MOD_RES 11: note = Xaa = glycine or proline
RXRXRXRRXRX

SEQ ID NO: 207 - protein, 11 aa, synthetic construct
  REGION 1..11: note = cell penetrating peptide
  MOD_RES 2: note = Xaa = beta-alanine
  MOD_RES 5..9: note = Xaa = 6-aminohexanoic acid
  MOD_RES 7: note = Xaa = -NH-(CHRe)n-C(O)-wherein n is 2 to 7 and each Re isindependently, at each occurrence, hydrogen or methyl
  MOD_RES 11: note = Xaa = glycine or proline
RXRRXRXRXRX

SEQ ID NO: 208 - protein, 15 aa, synthetic construct
  REGION 1..15: note = cell penetrating peptide
  MOD_RES 2: note = Xaa = beta-alanine
  MOD_RES 10..13: note = Xaa = 6-aminohexanoic acid
  MOD_RES 15: note = Xaa = glycine or proline
RXRILFQYRXRRXRX

SEQ ID NO: 209 - protein, 15 aa, synthetic construct
  REGION 1..15: note = cell penetrating peptide
  MOD_RES 2: note = Xaa = beta-alanine
  MOD_RES 5..13: note = Xaa = 6-aminohexanoic acid
  MOD_RES 15: note = Xaa = glycine or proline
RXRRXRILFQYRXRX

SEQ ID NO: 210 - protein, 12 aa, synthetic construct
  REGION 1..12: note = cell penetrating peptide
  MOD_RES 2: note = Xaa = beta-alanine
  MOD_RES 4..8: note = Xaa = -NH-(CHRe)n-C(O)-wherein n is 2 to 7 and each Re isindependently, at each occurrence, hydrogen or methyl
  MOD_RES 6..10: note = Xaa = 6-aminohexanoic acid
  MOD_RES 12: note = Xaa = glycine or proline
RXRXRXRXRXRX

SEQ ID NO: 211 - protein, 20 aa, synthetic construct
  REGION 1..20: note = cell penetrating peptide
  MOD_RES 2: note = Xaa = beta-alanine
  MOD_RES 10..18: note = Xaa = 6-aminohexanoic acid
  MOD_RES 20: note = Xaa = glycine or proline
RXRILFQYRXRILFQYRXRX

SEQ ID NO: 212 - protein, 16 aa, synthetic construct
  REGION 1..16: note = cell penetrating peptide
  MOD_RES 2: note = Xaa = beta-alanine
  MOD_RES 4: note = Xaa = -NH-(CHRe)n-C(O)-wherein n is 2 to 7 and each Re isindependently, at each occurrence, hydrogen or methyl
  MOD_RES 6..14: note = Xaa = 6-aminohexanoic acid
  MOD_RES 16: note = Xaa = glycine or proline
RXRXRXRILFQYRXRX

SEQ ID NO: 213 - protein, 16 aa, synthetic construct
  REGION 1..16: note = cell penetrating peptide
  MOD_RES 2: note = Xaa = beta-alanine
  MOD_RES 10..14: note = Xaa = 6-aminohexanoic acid
  MOD_RES 12: note = Xaa = -NH-(CHRe)n-C(O)-wherein n is 2 to 7 and each Re isindependently, at each occurrence, hydrogen or methyl
  MOD_RES 16: note = Xaa = glycine or proline
RXRILFQYRXRXRXRX

SEQ ID NO: 214 - protein, 11 aa, synthetic construct
  REGION 1..11: note = cell penetrating peptide
  MOD_RES 2..6: note = Xaa = beta-alanine
  MOD_RES 4: note = Xaa = -NH-(CHRe)n-C(O)-wherein n is 2 to 7 and each Re isindependently, at each occurrence, hydrogen or methyl
  MOD_RES 9: note = Xaa = 6-aminohexanoic acid
  MOD_RES 11: note = Xaa = glycine or proline
RXRXRXRRXRX

SEQ ID NO: 215 - protein, 11 aa, synthetic construct
  REGION 1..11: note = cell penetrating peptide
  MOD_RES 2..5: note = Xaa = beta-alanine
  MOD_RES 7: note = Xaa = -NH-(CHRe)n-C(O)-wherein n is 2 to 7 and each Re isindependently, at each occurrence, hydrogen or methyl
  MOD_RES 9: note = Xaa = 6-aminohexanoic acid
  MOD_RES 11: note = Xaa = glycine or proline
RXRRXRXRXRX

SEQ ID NO: 216 - protein, 15 aa, synthetic construct
  REGION 1..15: note = cell penetrating peptide
  MOD_RES 2..10: note = Xaa = beta-alanine
  MOD_RES 13: note = Xaa = 6-aminohexanoic acid
  MOD_RES 15: note = Xaa = glycine or proline
RXRILFQYRXRRXRX

SEQ ID NO: 217 - protein, 15 aa, synthetic construct
  REGION 1..15: note = cell penetrating peptide
  MOD_RES 2..5: note = Xaa = beta-alanine
  MOD_RES 13: note = Xaa = 6-aminohexanoic acid
  MOD_RES 15: note = Xaa = glycine or proline
RXRRXRILFQYRXRX

SEQ ID NO: 218 - protein, 12 aa, synthetic construct
  REGION 1..12: note = cell penetrating peptide
  MOD_RES 2..6: note = Xaa = beta-alanine
  MOD_RES 4..8: note = Xaa = -NH-(CHRe)n-C(O)-wherein n is 2 to 7 and each Re isindependently, at each occurrence, hydrogen or methyl
  MOD_RES 10: note = Xaa = 6-aminohexanoic acid
  MOD_RES 12: note = Xaa = glycine or proline
RXRXRXRXRXRX

SEQ ID NO: 219 - protein, 20 aa, synthetic construct
  REGION 1..20: note = cell penetrating peptide
  MOD_RES 2..10: note = Xaa = beta-alanine
  MOD_RES 18: note = Xaa = 6-aminohexanoic acid
  MOD_RES 20: note = Xaa = glycine or proline
RXRILFQYRXRILFQYRXRX

SEQ ID NO: 220 - protein, 16 aa, synthetic construct
  REGION 1..16: note = cell penetrating peptide
  MOD_RES 2..6: note = Xaa = beta-alanine
  MOD_RES 4: note = Xaa = -NH-(CHRe)n-C(O)-wherein n is 2 to 7 and each Re isindependently, at each occurrence, hydrogen or methyl
  MOD_RES 14: note = Xaa = 6-aminohexanoic acid
  MOD_RES 16: note = Xaa = glycine or proline
RXRXRXRILFQYRXRX

SEQ ID NO: 221 - protein, 16 aa, synthetic construct
  REGION 1..16: note = cell penetrating peptide
  MOD_RES 2..10: note = Xaa = beta-alanine
  MOD_RES 12: note = Xaa = -NH-(CHRe)n-C(O)-wherein n is 2 to 7 and each Re isindependently, at each occurrence, hydrogen or methyl
  MOD_RES 14: note = Xaa = 6-aminohexanoic acid
  MOD_RES 16: note = Xaa = glycine or proline
RXRILFQYRXRXRXRX

SEQ ID NO: 222 - protein, 13 aa, synthetic construct
  REGION 1..13: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  MOD_RES 13: note = Xaa = glycine or proline
RXRRXRRXRRXRX

SEQ ID NO: 223 - protein, 22 aa, synthetic construct
  REGION 1..22: note = cell penetrating peptide
  MOD_RES 2..8: note = Xaa = 6-aminohexanoic acid
  MOD_RES 5..18: note = Xaa = beta-alanine
  SITE 20: note = misc_feature - Xaa can be any naturally occurring amino acid
  MOD_RES 22: note = Xaa = glycine or proline
RXRRXRRXRILFQYRXRXRXRX

SEQ ID NO: 224 - protein, 13 aa, synthetic construct
  REGION 1..13: note = cell penetrating peptide
  MOD_RES 2..8: note = Xaa = 6-aminohexanoic acid
  MOD_RES 5..11: note = Xaa = beta-alanine
  MOD_RES 13: note = Xaa = glycine or proline
RXRRXRRXRRXRX

SEQ ID NO: 225 - protein, 13 aa, synthetic construct
  REGION 1..13: note = cell penetrating peptide
  MOD_RES 13: note = Xaa = glycine or proline
YGRKKRRQRRRPX

SEQ ID NO: 226 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 13: note = misc_feature - Xaa can be any naturally occurring amino acid
  MOD_RES 14: note = Xaa = beta-alanine
  SITE 22: note = misc_feature - Xaa can be any naturally occurring amino acid
  MOD_RES 24: note = Xaa = glycine or proline
RXRRXRRXRRXRXXASSLNIAXCX

SEQ ID NO: 227 - protein, 32 aa, synthetic construct
  REGION 1..32: note = cell penetrating peptide
  MOD_RES 2..8: note = Xaa = 6-aminohexanoic acid
  MOD_RES 5..18: note = Xaa = beta-alanine
  SITE 20: note = misc_feature - Xaa can be any naturally occurring amino acid
  SITE 22: note = misc_feature - Xaa can be any naturally occurring amino acid
  SITE 30: note = misc_feature - Xaa can be any naturally occurring amino acid
  MOD_RES 32: note = Xaa = glycine or proline
RXRRXRRXRILFQYRXRXRXRXASSLNIAXCX

SEQ ID NO: 228 - protein, 26 aa, synthetic construct
  REGION 1..26: note = cell penetrating peptide
  MOD_RES 2..8: note = Xaa = 6-aminohexanoic acid
  MOD_RES 5..20: note = Xaa = beta-alanine
  SITE 22: note = misc_feature - Xaa can be any naturally occurring amino acid
  SITE 24: note = misc_feature - Xaa can be any naturally occurring amino acid
  MOD_RES 26: note = Xaa = glycine or proline
RXRRXRRXRASSLNIARXRXRXRXCX

SEQ ID NO: 229 - protein, 22 aa, synthetic construct
  REGION 1..22: note = cell penetrating peptide
  MOD_RES 2..8: note = Xaa = 6-aminohexanoic acid
  MOD_RES 5..11: note = Xaa = beta-alanine
  REGION 13..14: note = misc_feature - Xaa can be any naturally occurring amino acid
  MOD_RES 22: note = Xaa = glycine or proline
RXRRXRRXRRXRXXASSLNIAX

SEQ ID NO: 230 - protein, 13 aa, synthetic construct
  REGION 1..13: note = cell penetrating peptide
  MOD_RES 13: note = Xaa = glycine or proline
THRPPMWSPVWPX

SEQ ID NO: 231 - protein, 12 aa, synthetic construct
  REGION 1..12: note = cell penetrating peptide
  MOD_RES 12: note = Xaa = glycine or proline
HRPPMWSPVWPX

SEQ ID NO: 232 - protein, 11 aa, synthetic construct
  REGION 1..11: note = cell penetrating peptide
  MOD_RES 11: note = Xaa = glycine or proline
THRPPMWSPVX

SEQ ID NO: 233 - protein, 10 aa, synthetic construct
  REGION 1..10: note = cell penetrating peptide
  MOD_RES 10: note = Xaa = glycine or proline
THRPPMWSPX

SEQ ID NO: 234 - protein, 13 aa, synthetic construct
  REGION 1..13: note = cell penetrating peptide
  MOD_RES 13: note = Xaa = glycine or proline
THRPPMWSPVFPX

SEQ ID NO: 235 - protein, 13 aa, synthetic construct
  REGION 1..13: note = cell penetrating peptide
  MOD_RES 13: note = Xaa = glycine or proline
THRPPMWSPVYPX

SEQ ID NO: 236 - protein, 13 aa, synthetic construct
  REGION 1..13: note = cell penetrating peptide
  MOD_RES 13: note = Xaa = glycine or proline
THRPPMWSPAWPX

SEQ ID NO: 237 - protein, 13 aa, synthetic construct
  REGION 1..13: note = cell penetrating peptide
  MOD_RES 13: note = Xaa = glycine or proline
THRPPMWSPLWPX

SEQ ID NO: 238 - protein, 13 aa, synthetic construct
  REGION 1..13: note = cell penetrating peptide
  MOD_RES 13: note = Xaa = glycine or proline
THRPPMWSPIWPX

SEQ ID NO: 239 - protein, 14 aa, synthetic construct
  REGION 1..14: note = cell penetrating peptide
  MOD_RES 14: note = Xaa = glycine or proline
THRPPMWTPVVWPX

SEQ ID NO: 240 - protein, 13 aa, synthetic construct
  REGION 1..13: note = cell penetrating peptide
  MOD_RES 13: note = Xaa = glycine or proline
THRPPMFSPVWPX

SEQ ID NO: 241 - protein, 9 aa, synthetic construct
  REGION 1..9: note = cell penetrating peptide
  MOD_RES 9: note = Xaa = glycine or proline
THRPPMWSX

SEQ ID NO: 242 - protein, 11 aa, synthetic construct
  REGION 1..11: note = cell penetrating peptide
  MOD_RES 11: note = Xaa = glycine or proline
HRPPMWSPVWX

SEQ ID NO: 243 - protein, 13 aa, synthetic construct
  REGION 1..13: note = cell penetrating peptide
  MOD_RES 13: note = Xaa = glycine or proline
THRPPMYSPVWPX

SEQ ID NO: 244 - protein, 13 aa, synthetic construct
  REGION 1..13: note = cell penetrating peptide
  MOD_RES 6: note = Xaa = norleucine
  MOD_RES 13: note = Xaa = glycine or proline
THRPPXWSPVWPX

SEQ ID NO: 245 - protein, 13 aa, synthetic construct
  REGION 1..13: note = cell penetrating peptide
  MOD_RES 13: note = Xaa = glycine or proline
THKPPMWSPVWPX

SEQ ID NO: 246 - protein, 13 aa, synthetic construct
  REGION 1..13: note = cell penetrating peptide
  MOD_RES 13: note = Xaa = glycine or proline
SHRPPMWSPVWPX

SEQ ID NO: 247 - protein, 8 aa, synthetic construct
  REGION 1..8: note = cell penetrating peptide
  MOD_RES 8: note = Xaa = glycine or proline
STFTHPRX

SEQ ID NO: 248 - protein, 8 aa, synthetic construct
  REGION 1..8: note = cell penetrating peptide
  MOD_RES 8: note = Xaa = glycine or proline
YDIDNRRX

SEQ ID NO: 249 - protein, 8 aa, synthetic construct
  REGION 1..8: note = cell penetrating peptide
  MOD_RES 8: note = Xaa = glycine or proline
AYKPVGRX

SEQ ID NO: 250 - protein, 8 aa, synthetic construct
  REGION 1..8: note = cell penetrating peptide
  MOD_RES 8: note = Xaa = glycine or proline
HAIYPRHX

SEQ ID NO: 251 - protein, 8 aa, synthetic construct
  REGION 1..8: note = cell penetrating peptide
  MOD_RES 8: note = Xaa = glycine or proline
HTPNSTHX

SEQ ID NO: 252 - protein, 8 aa, synthetic construct
  REGION 1..8: note = cell penetrating peptide
  MOD_RES 8: note = Xaa = glycine or proline
ASSPVHRX

SEQ ID NO: 253 - protein, 8 aa, synthetic construct
  REGION 1..8: note = cell penetrating peptide
  MOD_RES 8: note = Xaa = glycine or proline
SSLPLRKX

SEQ ID NO: 254 - protein, 5 aa, synthetic construct
  REGION 1..5: note = cell penetrating peptide
  MOD_RES 5: note = Xaa = glycine or proline
KKRSX

SEQ ID NO: 255 - protein, 5 aa, synthetic construct
  REGION 1..5: note = cell penetrating peptide
  MOD_RES 5: note = Xaa = glycine or proline
KRSKX

SEQ ID NO: 256 - protein, 6 aa, synthetic construct
  REGION 1..6: note = cell penetrating peptide
  MOD_RES 6: note = Xaa = glycine or proline
KKRSKX

SEQ ID NO: 257 - protein, 5 aa, synthetic construct
  REGION 1..5: note = cell penetrating peptide
  MOD_RES 5: note = Xaa = glycine or proline
KSRKX

SEQ ID NO: 258 - protein, 5 aa, synthetic construct
  REGION 1..5: note = cell penetrating peptide
  MOD_RES 5: note = Xaa = glycine or proline
SRKRX

SEQ ID NO: 259 - protein, 5 aa, synthetic construct
  REGION 1..5: note = cell penetrating peptide
  MOD_RES 5: note = Xaa = glycine or proline
RKRKX

SEQ ID NO: 260 - protein, 6 aa, synthetic construct
  REGION 1..6: note = cell penetrating peptide
  MOD_RES 6: note = Xaa = glycine or proline
KSRKRX

SEQ ID NO: 261 - protein, 8 aa, synthetic construct
  REGION 1..8: note = cell penetrating peptide
  MOD_RES 8: note = Xaa = glycine or proline
QHPPWRVX

SEQ ID NO: 262 - protein, 8 aa, synthetic construct
  REGION 1..8: note = cell penetrating peptide
  MOD_RES 8: note = Xaa = glycine or proline
THPPTTHX

SEQ ID NO: 263 - protein, 8 aa, synthetic construct
  REGION 1..8: note = cell penetrating peptide
  MOD_RES 8: note = Xaa = glycine or proline
YKHTPTTX

SEQ ID NO: 264 - protein, 8 aa, synthetic construct
  REGION 1..8: note = cell penetrating peptide
  MOD_RES 8: note = Xaa = glycine or proline
QGMHRGTX

SEQ ID NO: 265 - protein, 6 aa, synthetic construct
  REGION 1..6: note = cell penetrating peptide
  MOD_RES 6: note = Xaa = glycine or proline
SRKRKX

SEQ ID NO: 266 - protein, 7 aa, synthetic construct
  REGION 1..7: note = cell penetrating peptide
  MOD_RES 7: note = Xaa = glycine or proline
KSRKRKX

SEQ ID NO: 267 - protein, 8 aa, synthetic construct
  REGION 1..8: note = cell penetrating peptide
  MOD_RES 8: note = Xaa = glycine or proline
PKKKRKVX

SEQ ID NO: 268 - protein, 8 aa, synthetic construct
  REGION 1..8: note = cell penetrating peptide
  MOD_RES 8: note = Xaa = glycine or proline
GKKRSKVX

SEQ ID NO: 269 - protein, 8 aa, synthetic construct
  REGION 1..8: note = cell penetrating peptide
  MOD_RES 8: note = Xaa = glycine or proline
KSRKRKLX

SEQ ID NO: 270 - protein, 8 aa, synthetic construct
  REGION 1..8: note = cell penetrating peptide
  MOD_RES 8: note = Xaa = glycine or proline
HSPSKIPX

SEQ ID NO: 271 - protein, 8 aa, synthetic construct
  REGION 1..8: note = cell penetrating peptide
  MOD_RES 8: note = Xaa = glycine or proline
HMATFHYX

SEQ ID NO: 272 - protein, 8 aa, synthetic construct
  REGION 1..8: note = cell penetrating peptide
  MOD_RES 8: note = Xaa = glycine or proline
AQPNKFKX

SEQ ID NO: 273 - protein, 8 aa, synthetic construct
  REGION 1..8: note = cell penetrating peptide
  MOD_RES 8: note = Xaa = glycine or proline
NLTRLHTX

SEQ ID NO: 274 - protein, 5 aa, synthetic construct
  REGION 1..5: note = cell penetrating peptide
  MOD_RES 5: note = Xaa = glycine or proline
KKKRX

SEQ ID NO: 275 - protein, 5 aa, synthetic construct
  REGION 1..5: note = cell penetrating peptide
  MOD_RES 5: note = Xaa = glycine or proline
KKRKX

SEQ ID NO: 276 - protein, 6 aa, synthetic construct
  REGION 1..6: note = cell penetrating peptide
  MOD_RES 6: note = Xaa = glycine or proline
KKKRKX

SEQ ID NO: 277 - protein, 25 aa, synthetic construct
  REGION 1..25: note = cell penetrating peptide
  MOD_RES 25: note = Xaa = glycine or proline
RRRRRRQIKIWFQNRRMKWKKGGCX

SEQ ID NO: 278 - protein, 26 aa, synthetic construct
  REGION 1..26: note = cell penetrating peptide
  MOD_RES 26: note = Xaa = glycine or proline
RRRRRRRQIKIWFQNRRMKWKKGGCX

SEQ ID NO: 279 - protein, 20 aa, synthetic construct
  REGION 1..20: note = cell penetrating peptide
  MOD_RES 20: note = Xaa = glycine or proline
RQIKIWFQNRRMKWKKGGCX

SEQ ID NO: 280 - protein, 24 aa, synthetic construct
  REGION 1..24: note = cell penetrating peptide
  MOD_RES 24: note = Xaa = glycine or proline
RRRRRRRQIKIWFQNRRMKWKKCX

SEQ ID NO: 281 - protein, 29 aa, synthetic construct
  REGION 1..29: note = cell penetrating peptide
  MOD_RES 2..5: note = Xaa = 6-aminohexanoic acid
  SITE 8: note = misc_feature - Xaa can be any naturally occurring amino acid
  MOD_RES 29: note = Xaa = glycine or proline
RXRRXRRXRRQIKIWFQNRRMKWKKGGCX

SEQ ID NO: 282 - protein, 25 aa, synthetic construct
  REGION 1..25: note = cell penetrating peptide
  MOD_RES 17..19: note = Xaa = 6-aminohexanoic acid
  SITE 21: note = misc_feature - Xaa can be any naturally occurring amino acid
  SITE 23: note = misc_feature - Xaa can be any naturally occurring amino acid
  MOD_RES 25: note = Xaa = glycine or proline
RRRRRRRQIKILFQNRXRXRXRXCX

SEQ ID NO: 283 - protein, 14 aa, synthetic construct
  REGION 1..14: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  MOD_RES 14: note = Xaa = glycine or proline
RXRRXRRXRRXRCX

SEQ ID NO: 284 - protein, 15 aa, synthetic construct
  REGION 1..15: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 13: note = misc_feature - Xaa can be any naturally occurring amino acid
  MOD_RES 15: note = Xaa = glycine or proline
RXRRXRRXRRXRXCX

SEQ ID NO: 285 - protein, 27 aa, synthetic construct
  REGION 1..27: note = cell penetrating peptide
  MOD_RES 2..5: note = Xaa = 6-aminohexanoic acid
  SITE 8: note = misc_feature - Xaa can be any naturally occurring amino acid
  MOD_RES 27: note = Xaa = glycine or proline
RXRRXRRXRIKILFQNRRMKWKKGGCX

SEQ ID NO: 286 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  MOD_RES 25: note = Xaa = glycine or proline
RXRRXRRXRIKILFQNRRMKWKKCX

SEQ ID NO: 287 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  MOD_RES 24: note = Xaa = glycine or proline
RXRRXRRXRIKILFQNRMKWKKCX

SEQ ID NO: 288 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 25: note = Xaa = glycine or proline
RXRRXRRXRIKILFQNXRMKWKKCX

SEQ ID NO: 289 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  MOD_RES 25: note = Xaa = glycine or proline
RXRRXRRXRIKILFQNHRMKWKKCX

SEQ ID NO: 290 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 25: note = Xaa = glycine or proline
RXRRXRRXRIKILFQNXRMKWKKCX

SEQ ID NO: 291 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 25: note = Xaa = glycine or proline
RXRRXRRXRIKILFQNXRMKWKKCX

SEQ ID NO: 292 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 25: note = Xaa = glycine or proline
RXRRXRRXRIKILFQNXRMKWKACX

SEQ ID NO: 293 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIKILFQNXRMKWHKACX

SEQ ID NO: 294 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 25: note = Xaa = glycine or proline
RXRRXRRXRIKILFQNXRMKWHRCX

SEQ ID NO: 295 - protein, 25 aa, synthetic construct
  REGION 1..25: note = cell penetrating peptide
  MOD_RES 2..4: note = Xaa = 6-aminohexanoic acid
  SITE 6: note = misc_feature - Xaa can be any naturally occurring amino acid
  SITE 8: note = misc_feature - Xaa can be any naturally occurring amino acid
  MOD_RES 25: note = Xaa = glycine or proline
RXRXRXRXRIKILFQNRRMKWKKCX

SEQ ID NO: 296 - protein, 25 aa, synthetic construct
  REGION 1..25: note = cell penetrating peptide
  MOD_RES 25: note = Xaa = glycine or proline
RARARARARIKILFQNRRMKWKKCX

SEQ ID NO: 297 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIXILFQNXRMKWHKACX

SEQ ID NO: 298 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIHILFQNXRMKWHKACX

SEQ ID NO: 299 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIRILFQNXRMKWHKACX

SEQ ID NO: 300 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIXILFQYXRMKWHKACX

SEQ ID NO: 301 - protein, 27 aa, synthetic construct
  REGION 1..27: note = cell penetrating peptide
  MOD_RES 2..5: note = Xaa = 6-aminohexanoic acid
  SITE 8: note = misc_feature - Xaa can be any naturally occurring amino acid
  SITE 18: note = misc_feature - Xaa can be any naturally occurring amino acid
  MOD_RES 27: note = Xaa = glycine or proline
RXRRXRRXRLYSPLSFQXRMKWHKACX

SEQ ID NO: 302 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRISILFQYXRMKWHKACX

SEQ ID NO: 303 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 15: note = misc_feature - Xaa can be any naturally occurring amino acid
  MOD_RES 24: note = Xaa = glycine or proline
RXRRXRRXRILFQYXRMKWHKACX

SEQ ID NO: 304 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 14: note = misc_feature - Xaa can be any naturally occurring amino acid
  MOD_RES 23: note = Xaa = glycine or proline
RXRRXRIXILFQYXRMKWHKACX

SEQ ID NO: 305 - protein, 26 aa, synthetic construct
  REGION 1..26: note = cell penetrating peptide
  MOD_RES 2..8: note = Xaa = 6-aminohexanoic acid
  SITE 17: note = misc_feature - Xaa can be any naturally occurring amino acid
  MOD_RES 26: note = Xaa = glycine or proline
RXRRARRXRIHILFQYXRMKWHKACX

SEQ ID NO: 306 - protein, 26 aa, synthetic construct
  REGION 1..26: note = cell penetrating peptide
  MOD_RES 5..17: note = Xaa = 6-aminohexanoic acid
  MOD_RES 26: note = Xaa = glycine or proline
RARRXRRARIHILFQYXRMKWHKACX

SEQ ID NO: 307 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIHILFQYXRMKWHKACX

SEQ ID NO: 308 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIXILFQNXRMKWHKACX

SEQ ID NO: 309 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIHILFQNXRMKWHKACX

SEQ ID NO: 310 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  MOD_RES 24: note = Xaa = glycine or proline
RXRRXRRXRIKILFQNRRMKWHKX

SEQ ID NO: 311 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 24: note = Xaa = glycine or proline
RXRRXRRXRIKILFQNXRMKWHKX

SEQ ID NO: 312 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  MOD_RES 24: note = Xaa = glycine or proline
RXRRXRRXRIXILFQNRRMKWHKX

SEQ ID NO: 313 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 17: note = misc_feature - Xaa can be any naturally occurring amino acid
  MOD_RES 24: note = Xaa = glycine or proline
RXRRXRRXRIXILFQNXRMKWHKX

SEQ ID NO: 314 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  MOD_RES 24: note = Xaa = glycine or proline
RXRRXRRXRIHILFQNRRMKWHKX

SEQ ID NO: 315 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 24: note = Xaa = glycine or proline
RXRRXRRXRIHILFQNXRMKWHKX

SEQ ID NO: 316 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  MOD_RES 24: note = Xaa = glycine or proline
RXRRXRRXRIRILFQNRRMKWHKX

SEQ ID NO: 317 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 24: note = Xaa = glycine or proline
RXRRXRRXRIRILFQNXRMKWHKX

SEQ ID NO: 318 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  MOD_RES 23: note = Xaa = glycine or proline
RXRRXRRXRIILFQNRRMKWHKX

SEQ ID NO: 319 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 16: note = misc_feature - Xaa can be any naturally occurring amino acid
  MOD_RES 23: note = Xaa = glycine or proline
RXRRXRRXRIILFQNXRMKWHKX

SEQ ID NO: 320 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  MOD_RES 23: note = Xaa = glycine or proline
RXRRXRRXRKILFQNRRMKWHKX

SEQ ID NO: 321 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 16: note = misc_feature - Xaa can be any naturally occurring amino acid
  MOD_RES 23: note = Xaa = glycine or proline
RXRRXRRXRKILFQNXRMKWHKX

SEQ ID NO: 322 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 10: note = misc_feature - Xaa can be any naturally occurring amino acid
  MOD_RES 23: note = Xaa = glycine or proline
RXRRXRRXRXILFQNRRMKWHKX

SEQ ID NO: 323 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 10: note = misc_feature - Xaa can be any naturally occurring amino acid
  SITE 16: note = misc_feature - Xaa can be any naturally occurring amino acid
  MOD_RES 23: note = Xaa = glycine or proline
RXRRXRRXRXILFQNXRMKWHKX

SEQ ID NO: 324 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  MOD_RES 23: note = Xaa = glycine or proline
RXRRXRRXRHILFQNRRMKWHKX

SEQ ID NO: 325 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 16: note = misc_feature - Xaa can be any naturally occurring amino acid
  MOD_RES 23: note = Xaa = glycine or proline
RXRRXRRXRHILFQNXRMKWHKX

SEQ ID NO: 326 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  MOD_RES 23: note = Xaa = glycine or proline
RXRRXRRXRRILFQNRRMKWHKX

SEQ ID NO: 327 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 16: note = misc_feature - Xaa can be any naturally occurring amino acid
  MOD_RES 23: note = Xaa = glycine or proline
RXRRXRRXRRILFQNXRMKWHKX

SEQ ID NO: 328 - protein, 22 aa, synthetic construct
  REGION 1..22: note = cell penetrating peptide
  MOD_RES 2..5: note = Xaa = 6-aminohexanoic acid
  SITE 8: note = misc_feature - Xaa can be any naturally occurring amino acid
  MOD_RES 22: note = Xaa = glycine or proline
RXRRXRRXRILFQNRRMKWHKX

SEQ ID NO: 329 - protein, 22 aa, synthetic construct
  REGION 1..22: note = cell penetrating peptide
  MOD_RES 2..5: note = Xaa = 6-aminohexanoic acid
  SITE 8: note = misc_feature - Xaa can be any naturally occurring amino acid
  SITE 15: note = misc_feature - Xaa can be any naturally occurring amino acid
  MOD_RES 22: note = Xaa = glycine or proline
RXRRXRRXRILFQNXRMKWHKX

SEQ ID NO: 330 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  MOD_RES 24: note = Xaa = glycine or proline
RXRRXRRXRIKILFQYRRMKWHKX

SEQ ID NO: 331 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 24: note = Xaa = glycine or proline
RXRRXRRXRIKILFQYXRMKWHKX

SEQ ID NO: 332 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  MOD_RES 24: note = Xaa = glycine or proline
RXRRXRRXRIXILFQYRRMKWHKX

SEQ ID NO: 333 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 17: note = misc_feature - Xaa can be any naturally occurring amino acid
  MOD_RES 24: note = Xaa = glycine or proline
RXRRXRRXRIXILFQYXRMKWHKX

SEQ ID NO: 334 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  MOD_RES 24: note = Xaa = glycine or proline
RXRRXRRXRIHILFQYRRMKWHKX

SEQ ID NO: 335 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 24: note = Xaa = glycine or proline
RXRRXRRXRIHILFQYXRMKWHKX

SEQ ID NO: 336 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  MOD_RES 24: note = Xaa = glycine or proline
RXRRXRRXRIRILFQYRRMKWHKX

SEQ ID NO: 337 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 24: note = Xaa = glycine or proline
RXRRXRRXRIRILFQYXRMKWHKX

SEQ ID NO: 338 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  MOD_RES 23: note = Xaa - glycine or proline
RXRRXRRXRIILFQYRRMKWHKX

SEQ ID NO: 339 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 16: note = misc_feature - Xaa can be any naturally occurring amino acid
  MOD_RES 23: note = Xaa = glycine or proline
RXRRXRRXRIILFQYXRMKWHKX

SEQ ID NO: 340 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  MOD_RES 23: note = Xaa = glycine or proline
RXRRXRRXRKILFQYRRMKWHKX

SEQ ID NO: 341 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 16: note = misc_feature - Xaa can be any naturally occurring amino acid
  MOD_RES 23: note = Xaa = glycine or proline
RXRRXRRXRKILFQYXRMKWHKX

SEQ ID NO: 342 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 10: note = misc_feature - Xaa can be any naturally occurring amino acid
  MOD_RES 23: note = Xaa = glycine or proline
RXRRXRRXRXILFQYRRMKWHKX

SEQ ID NO: 343 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 10: note = misc_feature - Xaa can be any naturally occurring amino acid
  SITE 16: note = misc_feature - Xaa can be any naturally occurring amino acid
  MOD_RES 23: note = Xaa = glycine or proline
RXRRXRRXRXILFQYXRMKWHKX

SEQ ID NO: 344 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  MOD_RES 23: note = Xaa = glycine or proline
RXRRXRRXRHILFQYRRMKWHKX

SEQ ID NO: 345 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 16: note = misc_feature - Xaa can be any naturally occurring amino acid
  MOD_RES 23: note = Xaa = glycine or proline
RXRRXRRXRHILFQYXRMKWHKX

SEQ ID NO: 346 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  MOD_RES 23: note = Xaa = glycine or proline
RXRRXRRXRRILFQYRRMKWHKX

SEQ ID NO: 347 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 16: note = misc_feature - Xaa can be any naturally occurring amino acid
  MOD_RES 23: note = Xaa = glycine or proline
RXRRXRRXRRILFQYXRMKWHKX

SEQ ID NO: 348 - protein, 22 aa, synthetic construct
  REGION 1..22: note = cell penetrating peptide
  MOD_RES 2..5: note = Xaa = 6-aminohexanoic acid
  SITE 8: note = misc_feature - Xaa can be any naturally occurring amino acid
  MOD_RES 22: note = Xaa = glycine or proline
RXRRXRRXRILFQYRRMKWHKX

SEQ ID NO: 349 - protein, 22 aa, synthetic construct
  REGION 1..22: note = cell penetrating peptide
  MOD_RES 2..5: note = Xaa = 6-aminohexanoic acid
  SITE 8: note = misc_feature - Xaa can be any naturally occurring amino acid
  SITE 15: note = misc_feature - Xaa can be any naturally occurring amino acid
  MOD_RES 22: note = Xaa = glycine or proline
RXRRXRRXRILFQYXRMKWHKX

SEQ ID NO: 350 - protein, 10 aa, synthetic construct
  REGION 1..10: note = cell penetrating peptide
  MOD_RES 2..5: note = Xaa = 6-aminohexanoic acid
  SITE 8: note = misc_feature - Xaa can be any naturally occurring amino acid
  MOD_RES 10: note = Xaa = glycine or proline
RXRRXRRXRX

SEQ ID NO: 351 - protein, 13 aa, synthetic construct
  REGION 1..13: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  MOD_RES 13: note = Xaa = glycine or proline
RXRRXRRXRRXRX

SEQ ID NO: 352 - protein, 7 aa, synthetic construct
  REGION 1..7: note = cell penetrating peptide
  MOD_RES 7: note = Xaa = glycine or proline
RARRARX

SEQ ID NO: 353 - protein, 10 aa, synthetic construct
  REGION 1..10: note = cell penetrating peptide
  MOD_RES 10: note = Xaa = glycine or proline
RARRARRARX

SEQ ID NO: 354 - protein, 13 aa, synthetic construct
  REGION 1..13: note = cell penetrating peptide
  MOD_RES 13: note = Xaa = glycine or proline
RARRARRARRARX

SEQ ID NO: 355 - protein, 9 aa, synthetic construct
  REGION 1..9: note = cell penetrating peptide
  MOD_RES 2..5: note = Xaa = 6-aminohexanoic acid
  SITE 8: note = misc_feature - Xaa can be any naturally occurring amino acid
  MOD_RES 9: note = Xaa = glycine or proline
RXRRXRIXX

SEQ ID NO: 356 - protein, 10 aa, synthetic construct
  REGION 1..10: note = cell penetrating peptide
  MOD_RES 2..8: note = Xaa = 6-aminohexanoic acid
  MOD_RES 10: note = Xaa = glycine or proline
RXRRARRXRX

SEQ ID NO: 357 - protein, 10 aa, synthetic construct
  REGION 1..10: note = cell penetrating peptide
  MOD_RES 5: note = Xaa = 6-aminohexanoic acid
  MOD_RES 10: note = Xaa = glycine or proline
RARRXRRARX

SEQ ID NO: 358 - protein, 6 aa, synthetic construct
  REGION 1..6: note = cell penetrating peptide
  MOD_RES 6: note = Xaa = glycine or proline
RRRRRX

SEQ ID NO: 359 - protein, 7 aa, synthetic construct
  REGION 1..7: note = cell penetrating peptide
  MOD_RES 7: note = Xaa = glycine or proline
RRRRRRX

SEQ ID NO: 360 - protein, 8 aa, synthetic construct
  REGION 1..8: note = cell penetrating peptide
  MOD_RES 8: note = Xaa = glycine or proline
RRRRRRRX

SEQ ID NO: 361 - protein, 14 aa, synthetic construct
  REGION 1..14: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  MOD_RES 14: note = Xaa = glycine or proline
RXRRXRRXRRXRCX

SEQ ID NO: 362 - protein, 15 aa, synthetic construct
  REGION 1..15: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 13: note = misc_feature - Xaa can be any naturally occurring amino acid
  MOD_RES 15: note = Xaa = glycine or proline
RXRRXRRXRRXRXCX

SEQ ID NO: 363 - protein, 27 aa, synthetic construct
  REGION 1..27: note = cell penetrating peptide
  MOD_RES 2..5: note = Xaa = 6-aminohexanoic acid
  SITE 8: note = misc_feature - Xaa can be any naturally occurring amino acid
  MOD_RES 27: note = Xaa = glycine or proline
RXRRXRRXRIKILFQNRRMKWKKGGCX

SEQ ID NO: 364 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  MOD_RES 25: note = Xaa = glycine or proline
RXRRXRRXRIKILFQNRRMKWKKCX

SEQ ID NO: 365 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  MOD_RES 24: note = Xaa = glycine or proline
RXRRXRRXRIKILFQNRMKWKKCX

SEQ ID NO: 366 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 25: note = Xaa = glycine or proline
RXRRXRRXRIKILFQNXRMKWKKCX

SEQ ID NO: 367 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  MOD_RES 25: note = Xaa = glycine or proline
RXRRXRRXRIKILFQNHRMKWKKCX

SEQ ID NO: 368 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 25: note = Xaa = glycine or proline
RXRRXRRXRIKILFQNXRMKWKKCX

SEQ ID NO: 369 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 25: note = Xaa = glycine or proline
RXRRXRRXRIKILFQNXRMKWKKCX

SEQ ID NO: 370 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 25: note = Xaa = glycine or proline
RXRRXRRXRIKILFQNXRMKWKACX

SEQ ID NO: 371 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIKILFQNXRMKWHKACX

SEQ ID NO: 372 - protein, 25 aa, synthetic construct
  REGION 1..25: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 25: note = Xaa = glycine or proline
RXRRXRRXRIKILFQNXRMKWHRCX

SEQ ID NO: 373 - protein, 25 aa, synthetic construct
  REGION 1..25: note = cell penetrating peptide
  MOD_RES 2..4: note = Xaa = 6-aminohexanoic acid
  SITE 6: note = misc_feature - Xaa can be any naturally occurring amino acid
  SITE 8: note = misc_feature - Xaa can be any naturally occurring amino acid
  MOD_RES 25: note = Xaa = glycine or proline
RXRXRXRXRIKILFQNRRMKWKKCX

SEQ ID NO: 374 - protein, 25 aa, synthetic construct
  REGION 1..25: note = cell penetrating peptide
  MOD_RES 25: note = Xaa = glycine or proline
RARARARARIKILFQNRRMKWKKCX

SEQ ID NO: 375 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIXILFQNXRMKWHKACX

SEQ ID NO: 376 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIHILFQNXRMKWHKACX

SEQ ID NO: 377 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIRILFQNXRMKWHKACX

SEQ ID NO: 378 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIXILFQYXRMKWHKACX

SEQ ID NO: 379 - protein, 27 aa, synthetic construct
  REGION 1..27: note = cell penetrating peptide
  MOD_RES 2..5: note = Xaa = 6-aminohexanoic acid
  SITE 8: note = misc_feature - Xaa can be any naturally occurring amino acid
  SITE 18: note = misc_feature - Xaa can be any naturally occurring amino acid
  MOD_RES 27: note = Xaa = glycine or proline
RXRRXRRXRLYSPLSFQXRMKWHKACX

SEQ ID NO: 380 - protein, 8 aa, synthetic construct
  REGION 1..8: note = cell penetrating peptide
  MOD_RES 8: note = Xaa = glycine or proline
RRMKWHKX

SEQ ID NO: 381 - protein, 8 aa, synthetic construct
  REGION 1..8: note = cell penetrating peptide
  MOD_RES 1: note = Xaa = 6-aminohexanoic acid
  MOD_RES 8: note = Xaa = glycine or proline
XRMKWHKX

SEQ ID NO: 382 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILFQXXRMKWHKX

SEQ ID NO: 383 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILFQXXRMKWHKX

SEQ ID NO: 384 - protein, 26 aa, synthetic construct
  REGION 1..26: note = cell penetrating peptide
  MOD_RES 26: note = Xaa = glycine or proline
RRRRRRRQIKILFQNPKKKRKVGGCX

SEQ ID NO: 385 - protein, 17 aa, synthetic construct
  REGION 1..17: note = cell penetrating peptide
  MOD_RES 17: note = Xaa = glycine or proline
HHFFRRRRRRRRRFFCX

SEQ ID NO: 386 - protein, 19 aa, synthetic construct
  REGION 1..19: note = cell penetrating peptide
  MOD_RES 19: note = Xaa = glycine or proline
HHHHHHRRRRRRRRRFFCX

SEQ ID NO: 387 - protein, 21 aa, synthetic construct
  REGION 1..21: note = cell penetrating peptide
  MOD_RES 21: note = Xaa = glycine or proline
HHHHHHFFRRRRRRRRRFFCX

SEQ ID NO: 388 - protein, 20 aa, synthetic construct
  REGION 1..20: note = cell penetrating peptide
  MOD_RES 6..7: note = Xaa = 6-aminohexanoic acid
  MOD_RES 20: note = Xaa = glycine or proline
HHHHHXXRRRRRRRRRFFCX

SEQ ID NO: 389 - protein, 23 aa, synthetic construct
  REGION 1..23: note = cell penetrating peptide
  MOD_RES 7..8: note = Xaa = 6-aminohexanoic acid
  MOD_RES 23: note = Xaa = glycine or proline
HHHHHHXXFFRRRRRRRRRFFCX

SEQ ID NO: 390 - protein, 21 aa, synthetic construct
  REGION 1..21: note = cell penetrating peptide
  MOD_RES 4..16: note = Xaa = 6-aminohexanoic acid
  MOD_RES 21: note = Xaa = glycine or proline
HHHXRRRRRRRRRFFXHHHCX

SEQ ID NO: 391 - protein, 8 aa, synthetic construct
  REGION 1..8: note = cell penetrating peptide
  MOD_RES 1: note = Xaa = 6-aminohexanoic acid
  MOD_RES 8: note = Xaa = glycine or proline
XRMKWHKX

SEQ ID NO: 392 - protein, 8 aa, synthetic construct
  REGION 1..8: note = cell penetrating peptide
  MOD_RES 1: note = Xaa = 6-aminohexanoic acid
  MOD_RES 8: note = Xaa = glycine or proline
XRWKWHKX

SEQ ID NO: 393 - protein, 8 aa, synthetic construct
  REGION 1..8: note = cell penetrating peptide
  MOD_RES 2..6: note = Xaa = 6-aminohexanoic acid
  MOD_RES 8: note = Xaa = glycine or proline
RXRARXRX

SEQ ID NO: 394 - protein, 8 aa, synthetic construct
  REGION 1..8: note = cell penetrating peptide
  MOD_RES 2..4: note = Xaa = 6-aminohexanoic acid
  SITE 6: note = misc_feature - Xaa can be any naturally occurring amino acid
  MOD_RES 8: note = Xaa = glycine or proline
RXRXRXRX

SEQ ID NO: 395 - protein, 8 aa, synthetic construct
  REGION 1..8: note = cell penetrating peptide
  MOD_RES 4: note = Xaa = 6-aminohexanoic acid
  MOD_RES 8: note = Xaa = glycine or proline
RARXRARX

SEQ ID NO: 396 - protein, 6 aa, synthetic construct
  REGION 1..6: note = cell penetrating peptide
  MOD_RES 2: note = Xaa = 6-aminohexanoic acid
  MOD_RES 6: note = Xaa = glycine or proline
RXRARX

SEQ ID NO: 397 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILFQXXHMKWHKX

SEQ ID NO: 398 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILFQXXRWKWHKX

SEQ ID NO: 399 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILFQXXHWKWHKX

SEQ ID NO: 400 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 25: note = misc_feature - Xaa can be any naturally occurring amino acid
  MOD_RES 27: note = Xaa = glycine or proline
XXXXXXXXXXXXXXILFQXRXRARXRX

SEQ ID NO: 401 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 23: note = misc_feature - Xaa can be any naturally occurring amino acid
  SITE 25: note = misc_feature - Xaa can be any naturally occurring amino acid
  MOD_RES 27: note = Xaa = glycine or proline
XXXXXXXXXXXXXXILFQXRXRXRXRX

SEQ ID NO: 402 - protein, 26 aa, synthetic construct
  REGION 1..26: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 24: note = misc_feature - Xaa can be any naturally occurring amino acid
  MOD_RES 26: note = Xaa = glycine or proline
XXXXXXXXXXXXXXILFQXRXRRXRX

SEQ ID NO: 403 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 23: note = misc_feature - Xaa can be any naturally occurring amino acid
  MOD_RES 27: note = Xaa = glycine or proline
XXXXXXXXXXXXXXILFQXRARXRARX

SEQ ID NO: 404 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 25: note = misc_feature - Xaa can be any naturally occurring amino acid
  MOD_RES 27: note = Xaa = glycine or proline
XXXXXXXXXXXXXXILFQXRXRARXRX

SEQ ID NO: 405 - protein, 25 aa, synthetic construct
  REGION 1..25: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  MOD_RES 25: note = Xaa = glycine or proline
XXXXXXXXXXXXXXILFQXRXRARX

SEQ ID NO: 406 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILIQXXRMKWHKX

SEQ ID NO: 407 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILIQXXHMKWHKX

SEQ ID NO: 408 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILIQXXRWKWHKX

SEQ ID NO: 409 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILIQXXHWKWHKX

SEQ ID NO: 410 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 25: note = misc_feature - Xaa can be any naturally occurring amino acid
  MOD_RES 27: note = Xaa = glycine or proline
XXXXXXXXXXXXXXILIQXRXRARXRX

SEQ ID NO: 411 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 23: note = misc_feature - Xaa can be any naturally occurring amino acid
  SITE 25: note = misc_feature - Xaa can be any naturally occurring amino acid
  MOD_RES 27: note = Xaa = glycine or proline
XXXXXXXXXXXXXXILIQXRXRXRXRX

SEQ ID NO: 412 - protein, 26 aa, synthetic construct
  REGION 1..26: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 24: note = misc_feature - Xaa can be any naturally occurring amino acid
  MOD_RES 26: note = Xaa = glycine or proline
XXXXXXXXXXXXXXILIQXRXRRXRX

SEQ ID NO: 413 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 23: note = misc_feature - Xaa can be any naturally occurring amino acid
  MOD_RES 27: note = Xaa = glycine or proline
XXXXXXXXXXXXXXILIQXRARXRARX

SEQ ID NO: 414 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 25: note = misc_feature - Xaa can be any naturally occurring amino acid
  MOD_RES 27: note = Xaa = glycine or proline
XXXXXXXXXXXXXXILIQXRXRARXRX

SEQ ID NO: 415 - protein, 25 aa, synthetic construct
  REGION 1..25: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  MOD_RES 25: note = Xaa = glycine or proline
XXXXXXXXXXXXXXILIQXRXRARX

SEQ ID NO: 416 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILFQXXHMKWHKX

SEQ ID NO: 417 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILFQXXRWKWHKX

SEQ ID NO: 418 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILFQXXHWKWHKX

SEQ ID NO: 419 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 25: note = misc_feature - Xaa can be any naturally occurring amino acid
  MOD_RES 27: note = Xaa = glycine or proline
XXXXXXXXXXXXXXILFQXRXRARXRX

SEQ ID NO: 420 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 23: note = misc_feature - Xaa can be any naturally occurring amino acid
  SITE 25: note = misc_feature - Xaa can be any naturally occurring amino acid
  MOD_RES 27: note = Xaa = glycine or proline
XXXXXXXXXXXXXXILFQXRXRXRXRX

SEQ ID NO: 421 - protein, 26 aa, synthetic construct
  REGION 1..26: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 24: note = misc_feature - Xaa can be any naturally occurring amino acid
  MOD_RES 26: note = Xaa = glycine or proline
XXXXXXXXXXXXXXILFQXRXRRXRX

SEQ ID NO: 422 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 23: note = misc_feature - Xaa can be any naturally occurring amino acid
  MOD_RES 27: note = Xaa = glycine or proline
XXXXXXXXXXXXXXILFQXRARXRARX

SEQ ID NO: 423 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 25: note = misc_feature - Xaa can be any naturally occurring amino acid
  MOD_RES 27: note = Xaa = glycine or proline
XXXXXXXXXXXXXXILFQXRXRARXRX

SEQ ID NO: 424 - protein, 25 aa, synthetic construct
  REGION 1..25: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  MOD_RES 25: note = Xaa = glycine or proline
XXXXXXXXXXXXXXILFQXRXRARX

SEQ ID NO: 425 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILIQXXRMKWHKX

SEQ ID NO: 426 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILIQXXHMKWHKX

SEQ ID NO: 427 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILIQXXRWKWHKX

SEQ ID NO: 428 - protein, 27 aa, synthetic construct
  REGION 1..27: note = cell penetrating peptide
  MOD_RES 1..14: note = Xaa = 6-aminohexanoic acid
  REGION 19..20: note = misc_feature - Xaa can be any naturally occurring amino acid
  MOD_RES 27: note = Xaa = glycine or proline
XXXXXXXXXXXXXXILIQXXHWKWHKX

SEQ ID NO: 429 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 25: note = misc_feature - Xaa can be any naturally occurring amino acid
  MOD_RES 27: note = Xaa = glycine or proline
XXXXXXXXXXXXXXILIQXRXRARXRX

SEQ ID NO: 430 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 23: note = misc_feature - Xaa can be any naturally occurring amino acid
  SITE 25: note = misc_feature - Xaa can be any naturally occurring amino acid
  MOD_RES 27: note = Xaa = glycine or proline
XXXXXXXXXXXXXXILIQXRXRXRXRX

SEQ ID NO: 431 - protein, 26 aa, synthetic construct
  REGION 1..26: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 24: note = misc_feature - Xaa can be any naturally occurring amino acid
  MOD_RES 26: note = Xaa = glycine or proline
XXXXXXXXXXXXXXILIQXRXRRXRX

SEQ ID NO: 432 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 23: note = misc_feature - Xaa can be any naturally occurring amino acid
  MOD_RES 27: note = Xaa = glycine or proline
XXXXXXXXXXXXXXILIQXRARXRARX

SEQ ID NO: 433 - protein, 27 aa, synthetic construct
  REGION 1..27: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  SITE 25: note = misc_feature - Xaa can be any naturally occurring amino acid
  MOD_RES 27: note = Xaa = glycine or proline
XXXXXXXXXXXXXXILIQXRXRARXRX

SEQ ID NO: 434 - protein, 25 aa, synthetic construct
  REGION 1..25: note = cell penetrating peptide
  MOD_RES 1..14: note = Xaa = 6-aminohexanoic acid
  SITE 19: note = misc_feature - Xaa can be any naturally occurring amino acid
  SITE 21: note = misc_feature - Xaa can be any naturally occurring amino acid
  MOD_RES 25: note = Xaa = glycine or proline
XXXXXXXXXXXXXXILIQXRXRARX

SEQ ID NO: 435 - protein, 15 aa, synthetic construct
  REGION 1..15: note = cell penetrating peptide
  MOD_RES 2..8: note = Xaa = 6-aminohexanoic acid
  SITE 13: note = misc_feature - Xaa can be any naturally occurring amino acid
  MOD_RES 15: note = Xaa = glycine or proline
RXRRARRXRRARXAX

SEQ ID NO: 436 - protein, 24 aa, synthetic construct
  REGION 1..24: note = cell penetrating peptide
  MOD_RES 2..8: note = Xaa = 6-aminohexanoic acid
  SITE 15: note = misc_feature - Xaa can be any naturally occurring amino acid
  MOD_RES 24: note = Xaa = glycine or proline
RXRRARRXRILFQYXHMKWHKACX

SEQ ID NO: 437 - protein, 24 aa, synthetic construct
  REGION 1..24: note = cell penetrating peptide
  MOD_RES 2..8: note = Xaa = 6-aminohexanoic acid
  SITE 15: note = misc_feature - Xaa can be any naturally occurring amino acid
  MOD_RES 24: note = Xaa = glycine or proline
RXRRARRXRILFQYXRMKWHKACX

SEQ ID NO: 438 - protein, 24 aa, synthetic construct
  REGION 1..24: note = cell penetrating peptide
  MOD_RES 2..8: note = Xaa = 6-aminohexanoic acid
  SITE 15: note = misc_feature - Xaa can be any naturally occurring amino acid
  MOD_RES 24: note = Xaa = glycine or proline
RXRRARRXRILFQYXRWKWHKACX

SEQ ID NO: 439 - protein, 14 aa, synthetic construct
  REGION 1..14: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  MOD_RES 14: note = Xaa = glycine or proline
RXRRXRRXRRXRCX

SEQ ID NO: 440 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIXILFQNXRMKWHKACX

SEQ ID NO: 441 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIHILFQNXRMKWHKACX

SEQ ID NO: 442 - protein, 26 aa, synthetic construct
  REGION 1..26: note = cell penetrating peptide
  MOD_RES 2..5: note = Xaa = 6-aminohexanoic acid
  SITE 8: note = misc_feature - Xaa can be any naturally occurring amino acid
  SITE 11: note = misc_feature - Xaa can be any naturally occurring amino acid
  SITE 17: note = misc_feature - Xaa can be any naturally occurring amino acid
  MOD_RES 26: note = Xaa = glycine or proline
RXRRXRRXRIXILFQYXRMKWHKACX

SEQ ID NO: 443 - protein, 27 aa, synthetic construct
  REGION 1..27: note = cell penetrating peptide
  MOD_RES 2..5: note = Xaa = 6-aminohexanoic acid
  SITE 8: note = misc_feature - Xaa can be any naturally occurring amino acid
  SITE 18: note = misc_feature - Xaa can be any naturally occurring amino acid
  MOD_RES 27: note = Xaa = glycine or proline
RXRRXRRXRLYSPLSFQXRMKWHKACX

SEQ ID NO: 444 - protein, 24 aa, synthetic construct
  REGION 1..24: note = cell penetrating peptide
  MOD_RES 2..5: note = Xaa = 6-aminohexanoic acid
  SITE 8: note = misc_feature - Xaa can be any naturally occurring amino acid
  SITE 15: note = misc_feature - Xaa can be any naturally occurring amino acid
  MOD_RES 24: note = Xaa = glycine or proline
RXRRXRRXRILFQYXRMKWHKACX

SEQ ID NO: 445 - protein, 23 aa, synthetic construct
  REGION 1..23: note = cell penetrating peptide
  MOD_RES 2..5: note = Xaa = 6-aminohexanoic acid
  SITE 8: note = misc_feature - Xaa can be any naturally occurring amino acid
  SITE 14: note = misc_feature - Xaa can be any naturally occurring amino acid
  MOD_RES 23: note = Xaa = glycine or proline
RXRRXRIXILFQYXRMKWHKACX

SEQ ID NO: 446 - protein, 24 aa, synthetic construct
  REGION 1..24: note = cell penetrating peptide
  MOD_RES 5..15: note = Xaa = 6-aminohexanoic acid
  MOD_RES 24: note = Xaa = glycine or proline
RARRXRRARILFQYXRMKWHKACX

SEQ ID NO: 447 - protein, 24 aa, synthetic construct
  REGION 1..24: note = cell penetrating peptide
  MOD_RES 2..8: note = Xaa = 6-aminohexanoic acid
  SITE 15: note = misc_feature - Xaa can be any naturally occurring amino acid
  MOD_RES 24: note = Xaa = glycine or proline
RXRRARRXRILFQYXRMKWHKACX

SEQ ID NO: 448 - protein, 24 aa, synthetic construct
  REGION 1..24: note = cell penetrating peptide
  MOD_RES 5..15: note = Xaa = 6-aminohexanoic acid
  MOD_RES 24: note = Xaa = glycine or proline
RARRXRRARILFQYXRMKWHKACX

SEQ ID NO: 449 - protein, 24 aa, synthetic construct
  REGION 1..24: note = cell penetrating peptide
  MOD_RES 2..8: note = Xaa = 6-aminohexanoic acid
  SITE 15: note = misc_feature - Xaa can be any naturally occurring amino acid
  MOD_RES 24: note = Xaa = glycine or proline
RXRRARRXRILFQYXRMKWHKACX

SEQ ID NO: 450 - protein, 24 aa, synthetic construct
  REGION 1..24: note = cell penetrating peptide
  MOD_RES 2..8: note = Xaa = 6-aminohexanoic acid
  SITE 15: note = misc_feature - Xaa can be any naturally occurring amino acid
  MOD_RES 24: note = Xaa = glycine or proline
RXRRARRXRILFQYXHMKWHKACX

SEQ ID NO: 451 - protein, 24 aa, synthetic construct
  REGION 1..24: note = cell penetrating peptide
  MOD_RES 2..8: note = Xaa = 6-aminohexanoic acid
  SITE 15: note = misc_feature - Xaa can be any naturally occurring amino acid
  MOD_RES 24: note = Xaa = glycine or proline
RXRRARRXRILFQYXRMKWHKACX

SEQ ID NO: 452 - protein, 24 aa, synthetic construct
  REGION 1..24: note = cell penetrating peptide
  MOD_RES 2..8: note = Xaa = 6-aminohexanoic acid
  SITE 15: note = misc_feature - Xaa can be any naturally occurring amino acid
  MOD_RES 24: note = Xaa = glycine or proline
RXRRARRXRILFQYXRWKWHKACX

SEQ ID NO: 453 - protein, 24 aa, synthetic construct
  REGION 1..24: note = cell penetrating peptide
  MOD_RES 2..8: note = Xaa = 6-aminohexanoic acid
  SITE 15: note = misc_feature - Xaa can be any naturally occurring amino acid
  MOD_RES 24: note = Xaa = glycine or proline
RXRRARRXRILFQYXHWKWHKACX

SEQ ID NO: 454 - protein, 24 aa, synthetic construct
  REGION 1..24: note = cell penetrating peptide
  MOD_RES 2..8: note = Xaa = 6-aminohexanoic acid
  SITE 16: note = misc_feature - Xaa can be any naturally occurring amino acid
  SITE 20: note = misc_feature - Xaa can be any naturally occurring amino acid
  MOD_RES 24: note = Xaa = glycine or proline
RXRRARRXRILFQYRXRARXRACX

SEQ ID NO: 455 - protein, 24 aa, synthetic construct
  REGION 1..24: note = cell penetrating peptide
  MOD_RES 2..8: note = Xaa = 6-aminohexanoic acid
  SITE 16: note = misc_feature - Xaa can be any naturally occurring amino acid
  SITE 18: note = misc_feature - Xaa can be any naturally occurring amino acid
  SITE 20: note = misc_feature - Xaa can be any naturally occurring amino acid
  MOD_RES 24: note = Xaa = glycine or proline
RXRRARRXRILFQYRXRXRXRACX

SEQ ID NO: 456 - protein, 24 aa, synthetic construct
  REGION 1..24: note = cell penetrating peptide
  MOD_RES 2..8: note = Xaa = 6-aminohexanoic acid
  SITE 15: note = misc_feature - Xaa can be any naturally occurring amino acid
  MOD_RES 24: note = Xaa = glycine or proline
RXRRARRXRILIQYXRMKWHKACX

SEQ ID NO: 457 - protein, 19 aa, synthetic construct
  REGION 1..19: note = cell penetrating peptide
  MOD_RES 2..5: note = Xaa = 6-aminohexanoic acid
  SITE 13: note = misc_feature - Xaa can be any naturally occurring amino acid
  SITE 16: note = misc_feature - Xaa can be any naturally occurring amino acid
  MOD_RES 19: note = Xaa = glycine or proline
RXRRXRILFQYRXRRXRCX

SEQ ID NO: 458 - protein, 24 aa, synthetic construct
  REGION 1..24: note = cell penetrating peptide
  MOD_RES 2..8: note = Xaa = 6-aminohexanoic acid
  SITE 16: note = misc_feature - Xaa can be any naturally occurring amino acid
  SITE 20: note = misc_feature - Xaa can be any naturally occurring amino acid
  MOD_RES 24: note = Xaa = glycine or proline
RXRRARRXRILFQYRXRARXRACX

SEQ ID NO: 459 - protein, 24 aa, synthetic construct
  REGION 1..24: note = cell penetrating peptide
  MOD_RES 2..8: note = Xaa = 6-aminohexanoic acid
  SITE 16: note = misc_feature - Xaa can be any naturally occurring amino acid
  SITE 18: note = misc_feature - Xaa can be any naturally occurring amino acid
  SITE 20: note = misc_feature - Xaa can be any naturally occurring amino acid
  MOD_RES 24: note = Xaa = glycine or proline
RXRRARRXRILFQYRXRXRXRACX

SEQ ID NO: 460 - protein, 24 aa, synthetic construct
  REGION 1..24: note = cell penetrating peptide
  MOD_RES 2..8: note = Xaa = 6-aminohexanoic acid
  SITE 15: note = misc_feature - Xaa can be any naturally occurring amino acid
  MOD_RES 24: note = Xaa = glycine or proline
RXRRARRXRILIQYXRMKWHKACX

SEQ ID NO: 461 - protein, 21 aa, synthetic construct
  REGION 1..21: note = cell penetrating peptide
  MOD_RES 2..5: note = Xaa = 6-aminohexanoic acid
  SITE 13: note = misc_feature - Xaa can be any naturally occurring amino acid
  SITE 16: note = misc_feature - Xaa can be any naturally occurring amino acid
  MOD_RES 21: note = Xaa = glycine or proline
RXRRXRILFQYRXRRXRCYSX

SEQ ID NO: 462 - protein, 24 aa, synthetic construct
  REGION 1..24: note = cell penetrating peptide
  MOD_RES 5..18: note = Xaa = 6-aminohexanoic acid
  MOD_RES 24: note = Xaa = glycine or proline
RARRXRRARILFQYRARXRARACX

SEQ ID NO: 463 - protein, 24 aa, synthetic construct
  REGION 1..24: note = cell penetrating peptide
  MOD_RES 5..16: note = Xaa = 6-aminohexanoic acid
  SITE 20: note = misc_feature - Xaa can be any naturally occurring amino acid
  MOD_RES 24: note = Xaa = glycine or proline
RARRXRRARILFQYRXRARXRACX

SEQ ID NO: 464 - protein, 23 aa, synthetic construct
  REGION 1..23: note = cell penetrating peptide
  MOD_RES 5..16: note = Xaa = 6-aminohexanoic acid
  SITE 19: note = misc_feature - Xaa can be any naturally occurring amino acid
  MOD_RES 23: note = Xaa = glycine or proline
RARRXRRARILFQYRXRRXRACX

SEQ ID NO: 465 - protein, 23 aa, synthetic construct
  REGION 1..23: note = cell penetrating peptide
  MOD_RES 5..16: note = Xaa = 6-aminohexanoic acid
  SITE 20: note = misc_feature - Xaa can be any naturally occurring amino acid
  MOD_RES 23: note = Xaa = glycine or proline
RARRXRRARILFQYRXRARXACX

SEQ ID NO: 466 - protein, 23 aa, synthetic construct
  REGION 1..23: note = cell penetrating peptide
  MOD_RES 2..8: note = Xaa = 6-aminohexanoic acid
  SITE 16: note = misc_feature - Xaa can be any naturally occurring amino acid
  SITE 19: note = misc_feature - Xaa can be any naturally occurring amino acid
  MOD_RES 23: note = Xaa = glycine or proline
RXRRARRXRILFQYRXRRXRACX

SEQ ID NO: 467 - protein, 23 aa, synthetic construct
  REGION 1..23: note = cell penetrating peptide
  MOD_RES 2..8: note = Xaa = 6-aminohexanoic acid
  SITE 16: note = misc_feature - Xaa can be any naturally occurring amino acid
  SITE 20: note = misc_feature - Xaa can be any naturally occurring amino acid
  MOD_RES 23: note = Xaa = glycine or proline
RXRRARRXRILFQYRXRARXACX

SEQ ID NO: 468 - protein, 26 aa, synthetic construct
  REGION 1..26: note = cell penetrating peptide
  MOD_RES 2..8: note = Xaa = 6-aminohexanoic acid
  SITE 17: note = misc_feature - Xaa can be any naturally occurring amino acid
  MOD_RES 26: note = Xaa = glycine or proline
RXRRARRXRIHILFQNXRMKWHKACX

SEQ ID NO: 469 - protein, 16 aa, synthetic construct
  REGION 1..16: note = cell penetrating peptide
  MOD_RES 2..8: note = Xaa = 6-aminohexanoic acid
  SITE 13: note = misc_feature - Xaa can be any naturally occurring amino acid
  MOD_RES 16: note = Xaa = glycine or proline
RXRRARRXRRARXACX

SEQ ID NO: 470 - protein, 22 aa, synthetic construct
  REGION 1..22: note = cell penetrating peptide
  MOD_RES 2..8: note = Xaa = 6-aminohexanoic acid
  SITE 15: note = misc_feature - Xaa can be any naturally occurring amino acid
  MOD_RES 22: note = Xaa = glycine or proline
RXRRARRXRILFQYXHMKWHKX

SEQ ID NO: 471 - protein, 22 aa, synthetic construct
  REGION 1..22: note = cell penetrating peptide
  MOD_RES 2..8: note = Xaa = 6-aminohexanoic acid
  SITE 15: note = misc_feature - Xaa can be any naturally occurring amino acid
  MOD_RES 22: note = Xaa = glycine or proline
RXRRARRXRILFQYXRMKWHKX

SEQ ID NO: 472 - protein, 22 aa, synthetic construct
  REGION 1..22: note = cell penetrating peptide
  MOD_RES 2..8: note = Xaa = 6-aminohexanoic acid
  SITE 15: note = misc_feature - Xaa can be any naturally occurring amino acid
  MOD_RES 22: note = Xaa = glycine or proline
RXRRARRXRILFQYXRWKWHKX

SEQ ID NO: 473 - protein, 22 aa, synthetic construct
  REGION 1..22: note = cell penetrating peptide
  MOD_RES 2..8: note = Xaa = 6-aminohexanoic acid
  SITE 15: note = misc_feature - Xaa can be any naturally occurring amino acid
  MOD_RES 22: note = Xaa = glycine or proline
RXRRARRXRILFQYXRMKWHKX

SEQ ID NO: 474 - protein, 22 aa, synthetic construct
  REGION 1..22: note = cell penetrating peptide
  MOD_RES 2..8: note = Xaa = 6-aminohexanoic acid
  SITE 16: note = misc_feature - Xaa can be any naturally occurring amino acid
  SITE 20: note = misc_feature - Xaa can be any naturally occurring amino acid
  MOD_RES 22: note = Xaa = glycine or proline
RXRRARRXRILFQYRXRARXRX

SEQ ID NO: 475 - protein, 22 aa, synthetic construct
  REGION 1..22: note = cell penetrating peptide
  MOD_RES 2..8: note = Xaa = 6-aminohexanoic acid
  SITE 16: note = misc_feature - Xaa can be any naturally occurring amino acid
  SITE 18: note = misc_feature - Xaa can be any naturally occurring amino acid
  SITE 20: note = misc_feature - Xaa can be any naturally occurring amino acid
  MOD_RES 22: note = Xaa = glycine or proline
RXRRARRXRILFQYRXRXRXRX

SEQ ID NO: 476 - protein, 21 aa, synthetic construct
  REGION 1..21: note = cell penetrating peptide
  MOD_RES 2..8: note = Xaa = 6-aminohexanoic acid
  SITE 16: note = misc_feature - Xaa can be any naturally occurring amino acid
  SITE 19: note = misc_feature - Xaa can be any naturally occurring amino acid
  MOD_RES 21: note = Xaa = glycine or proline
RXRRARRXRILFQYRXRRXRX

SEQ ID NO: 477 - protein, 22 aa, synthetic construct
  REGION 1..22: note = cell penetrating peptide
  MOD_RES 2..8: note = Xaa = 6-aminohexanoic acid
  SITE 18: note = misc_feature - Xaa can be any naturally occurring amino acid
  MOD_RES 22: note = Xaa = glycine or proline
RXRRARRXRILFQYRARXRARX

SEQ ID NO: 478 - protein, 20 aa, synthetic construct
  REGION 1..20: note = cell penetrating peptide
  MOD_RES 2..8: note = Xaa = 6-aminohexanoic acid
  SITE 16: note = misc_feature - Xaa can be any naturally occurring amino acid
  MOD_RES 20: note = Xaa = glycine or proline
RXRRARRXRILFQYRXRARX

SEQ ID NO: 479 - protein, 22 aa, synthetic construct
  REGION 1..22: note = cell penetrating peptide
  MOD_RES 2..8: note = Xaa = 6-aminohexanoic acid
  SITE 15: note = misc_feature - Xaa can be any naturally occurring amino acid
  MOD_RES 22: note = Xaa = glycine or proline
RXRRARRXRILIQYXHMKWHKX

SEQ ID NO: 480 - protein, 22 aa, synthetic construct
  REGION 1..22: note = cell penetrating peptide
  MOD_RES 2..8: note = Xaa = 6-aminohexanoic acid
  SITE 15: note = misc_feature - Xaa can be any naturally occurring amino acid
  MOD_RES 22: note = Xaa = glycine or proline
RXRRARRXRILIQYXRMKWHKX

SEQ ID NO: 481 - protein, 22 aa, synthetic construct
  REGION 1..22: note = cell penetrating peptide
  MOD_RES 2..8: note = Xaa = 6-aminohexanoic acid
  SITE 15: note = misc_feature - Xaa can be any naturally occurring amino acid
  MOD_RES 22: note = Xaa = glycine or proline
RXRRARRXRILIQYXRWKWHKX

SEQ ID NO: 482 - protein, 22 aa, synthetic construct
  REGION 1..22: note = cell penetrating peptide
  MOD_RES 2..8: note = Xaa = 6-aminohexanoic acid
  SITE 15: note = misc_feature - Xaa can be any naturally occurring amino acid
  MOD_RES 22: note = Xaa = glycine or proline
RXRRARRXRILIQYXRMKWHKX

SEQ ID NO: 483 - protein, 22 aa, synthetic construct
  REGION 1..22: note = cell penetrating peptide
  MOD_RES 2..8: note = Xaa = 6-aminohexanoic acid
  SITE 16: note = misc_feature - Xaa can be any naturally occurring amino acid
  SITE 20: note = misc_feature - Xaa can be any naturally occurring amino acid
  MOD_RES 22: note = Xaa = glycine or proline
RXRRARRXRILIQYRXRARXRX

SEQ ID NO: 484 - protein, 22 aa, synthetic construct
  REGION 1..22: note = cell penetrating peptide
  MOD_RES 2..8: note = Xaa = 6-aminohexanoic acid
  SITE 16: note = misc_feature - Xaa can be any naturally occurring amino acid
  SITE 18: note = misc_feature - Xaa can be any naturally occurring amino acid
  SITE 20: note = misc_feature - Xaa can be any naturally occurring amino acid
  MOD_RES 22: note = Xaa = glycine or proline
RXRRARRXRILIQYRXRXRXRX

SEQ ID NO: 485 - protein, 21 aa, synthetic construct
  REGION 1..21: note = cell penetrating peptide
  MOD_RES 2..8: note = Xaa = 6-aminohexanoic acid
  SITE 16: note = misc_feature - Xaa can be any naturally occurring amino acid
  SITE 19: note = misc_feature - Xaa can be any naturally occurring amino acid
  MOD_RES 21: note = Xaa = glycine or proline
RXRRARRXRILIQYRXRRXRX

SEQ ID NO: 486 - protein, 22 aa, synthetic construct
  REGION 1..22: note = cell penetrating peptide
  MOD_RES 2..8: note = Xaa = 6-aminohexanoic acid
  SITE 18: note = misc_feature - Xaa can be any naturally occurring amino acid
  MOD_RES 22: note = Xaa = glycine or proline
RXRRARRXRILIQYRARXRARX

SEQ ID NO: 487 - protein, 20 aa, synthetic construct
  REGION 1..20: note = cell penetrating peptide
  MOD_RES 2..8: note = Xaa = 6-aminohexanoic acid
  SITE 16: note = misc_feature - Xaa can be any naturally occurring amino acid
  MOD_RES 20: note = Xaa = glycine or proline
RXRRARRXRILIQYRXRARX

SEQ ID NO: 488 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILFQYXHMKWHKX

SEQ ID NO: 489 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILFQYXRMKWHKX

SEQ ID NO: 490 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILFQYXRWKWHKX

SEQ ID NO: 491 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILFQYXRMKWHKX

SEQ ID NO: 492 - protein, 22 aa, synthetic construct
  REGION 1..22: note = cell penetrating peptide
  MOD_RES 5..16: note = Xaa = 6-aminohexanoic acid
  SITE 20: note = misc_feature - Xaa can be any naturally occurring amino acid
  MOD_RES 22: note = Xaa = glycine or proline
RARRXRRARILFQYRXRARXRX

SEQ ID NO: 493 - protein, 22 aa, synthetic construct
  REGION 1..22: note = cell penetrating peptide
  MOD_RES 5..16: note = Xaa = 6-aminohexanoic acid
  SITE 18: note = misc_feature - Xaa can be any naturally occurring amino acid
  SITE 20: note = misc_feature - Xaa can be any naturally occurring amino acid
  MOD_RES 22: note = Xaa = glycine or proline
RARRXRRARILFQYRXRXRXRX

SEQ ID NO: 494 - protein, 21 aa, synthetic construct
  REGION 1..21: note = cell penetrating peptide
  MOD_RES 5..16: note = Xaa = 6-aminohexanoic acid
  SITE 19: note = misc_feature - Xaa can be any naturally occurring amino acid
  MOD_RES 21: note = Xaa = glycine or proline
RARRXRRARILFQYRXRRXRX

SEQ ID NO: 495 - protein, 22 aa, synthetic construct
  REGION 1..22: note = cell penetrating peptide
  MOD_RES 5..18: note = Xaa = 6-aminohexanoic acid
  MOD_RES 22: note = Xaa = glycine or proline
RARRXRRARILFQYRARXRARX

SEQ ID NO: 496 - protein, 20 aa, synthetic construct
  REGION 1..20: note = cell penetrating peptide
  MOD_RES 5..16: note = Xaa = 6-aminohexanoic acid
  MOD_RES 20: note = Xaa = glycine or proline
RARRXRRARILFQYRXRARX

SEQ ID NO: 497 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILIQYXHMKWHKX

SEQ ID NO: 498 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILIQYXRMKWHKX

SEQ ID NO: 499 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILIQYXRWKWHKX

SEQ ID NO: 500 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILIQYXRMKWHKX

SEQ ID NO: 501 - protein, 22 aa, synthetic construct
  REGION 1..22: note = cell penetrating peptide
  MOD_RES 5..16: note = Xaa = 6-aminohexanoic acid
  SITE 20: note = misc_feature - Xaa can be any naturally occurring amino acid
  MOD_RES 22: note = Xaa = glycine or proline
RARRXRRARILIQYRXRARXRX

SEQ ID NO: 502 - protein, 22 aa, synthetic construct
  REGION 1..22: note = cell penetrating peptide
  MOD_RES 5..16: note = Xaa = 6-aminohexanoic acid
  SITE 18: note = misc_feature - Xaa can be any naturally occurring amino acid
  SITE 20: note = misc_feature - Xaa can be any naturally occurring amino acid
  MOD_RES 22: note = Xaa = glycine or proline
RARRXRRARILIQYRXRXRXRX

SEQ ID NO: 503 - protein, 21 aa, synthetic construct
  REGION 1..21: note = cell penetrating peptide
  MOD_RES 5..16: note = Xaa = 6-aminohexanoic acid
  SITE 19: note = misc_feature - Xaa can be any naturally occurring amino acid
  MOD_RES 21: note = Xaa = glycine or proline
RARRXRRARILIQYRXRRXRX

SEQ ID NO: 504 - protein, 22 aa, synthetic construct
  REGION 1..22: note = cell penetrating peptide
  MOD_RES 5..18: note = Xaa = 6-aminohexanoic acid
  MOD_RES 22: note = Xaa = glycine or proline
RARRXRRARILIQYRARXRARX

SEQ ID NO: 505 - protein, 20 aa, synthetic construct
  REGION 1..20: note = cell penetrating peptide
  MOD_RES 5..16: note = Xaa = 6-aminohexanoic acid
  MOD_RES 20: note = Xaa = glycine or proline
RARRXRRARILIQYRXRARX

SEQ ID NO: 506 - protein, 19 aa, synthetic construct
  REGION 1..19: note = cell penetrating peptide
  MOD_RES 2..5: note = Xaa = glycine or proline
  SITE 12: note = misc_feature - Xaa can be any naturally occurring amino acid
  SITE 19: note = misc_feature - Xaa can be any naturally occurring amino acid
RXRRXRILFQYXHMKWHKX

SEQ ID NO: 507 - protein, 19 aa, synthetic construct
  REGION 1..19: note = cell penetrating peptide
  MOD_RES 2..5: note = Xaa = 6-aminohexanoic acid
  SITE 12: note = misc_feature - Xaa can be any naturally occurring amino acid
  MOD_RES 19: note = Xaa = glycine or proline
RXRRXRILFQYXRMKWHKX

SEQ ID NO: 508 - protein, 19 aa, synthetic construct
  REGION 1..19: note = cell penetrating peptide
  MOD_RES 2..5: note = Xaa = 6-aminohexanoic acid
  SITE 12: note = misc_feature - Xaa can be any naturally occurring amino acid
  MOD_RES 19: note = Xaa = glycine or proline
RXRRXRILFQYXRWKWHKX

SEQ ID NO: 509 - protein, 19 aa, synthetic construct
  REGION 1..19: note = cell penetrating peptide
  MOD_RES 2..5: note = Xaa = 6-aminohexanoic acid
  SITE 12: note = misc_feature - Xaa can be any naturally occurring amino acid
  MOD_RES 19: note = Xaa = glycine or proline
RXRRXRILFQYXRMKWHKX

SEQ ID NO: 510 - protein, 19 aa, synthetic construct
  REGION 1..19: note = cell penetrating peptide
  MOD_RES 2..5: note = Xaa = 6-aminohexanoic acid
  SITE 13: note = misc_feature - Xaa can be any naturally occurring amino acid
  SITE 17: note = misc_feature - Xaa can be any naturally occurring amino acid
  MOD_RES 19: note = Xaa = glycine or proline
RXRRXRILFQYRXRARXRX

SEQ ID NO: 511 - protein, 19 aa, synthetic construct
  REGION 1..19: note = cell penetrating peptide
  MOD_RES 2..5: note = Xaa = 6-aminohexanoic acid
  SITE 13: note = misc_feature - Xaa can be any naturally occurring amino acid
  SITE 15: note = misc_feature - Xaa can be any naturally occurring amino acid
  SITE 17: note = misc_feature - Xaa can be any naturally occurring amino acid
  MOD_RES 19: note = Xaa = glycine or proline
RXRRXRILFQYRXRXRXRX

SEQ ID NO: 512 - protein, 18 aa, synthetic construct
  REGION 1..18: note = cell penetrating peptide
  MOD_RES 2..5: note = Xaa = 6-aminohexanoic acid
  SITE 13: note = misc_feature - Xaa can be any naturally occurring amino acid
  SITE 16: note = misc_feature - Xaa can be any naturally occurring amino acid
  MOD_RES 18: note = Xaa = glycine or proline
RXRRXRILFQYRXRRXRX

SEQ ID NO: 513 - protein, 19 aa, synthetic construct
  REGION 1..19: note = cell penetrating peptide
  MOD_RES 2..5: note = Xaa = 6-aminohexanoic acid
  SITE 15: note = misc_feature - Xaa can be any naturally occurring amino acid
  MOD_RES 19: note = Xaa = glycine or proline
RXRRXRILFQYRARXRARX

SEQ ID NO: 514 - protein, 17 aa, synthetic construct
  REGION 1..17: note = cell penetrating peptide
  MOD_RES 2..5: note = Xaa = 6-aminohexanoic acid
  SITE 13: note = misc_feature - Xaa can be any naturally occurring amino acid
  MOD_RES 17: note = Xaa = glycine or proline
RXRRXRILFQYRXRARX

SEQ ID NO: 515 - protein, 19 aa, synthetic construct
  REGION 1..19: note = cell penetrating peptide
  MOD_RES 2..5: note = Xaa = 6-aminohexanoic acid
  SITE 12: note = misc_feature - Xaa can be any naturally occurring amino acid
  MOD_RES 19: note = Xaa = glycine or proline
RXRRXRILIQYXHMKWHKX

SEQ ID NO: 516 - protein, 19 aa, synthetic construct
  REGION 1..19: note = cell penetrating peptide
  MOD_RES 2..5: note = Xaa = 6-aminohexanoic acid
  SITE 12: note = misc_feature - Xaa can be any naturally occurring amino acid
  MOD_RES 19: note = Xaa = glycine or proline
RXRRXRILIQYXRMKWHKX

SEQ ID NO: 517 - protein, 19 aa, synthetic construct
  REGION 1..19: note = cell penetrating peptide
  MOD_RES 2..5: note = Xaa = 6-aminohexanoic acid
  SITE 12: note = misc_feature - Xaa can be any naturally occurring amino acid
  MOD_RES 19: note = Xaa = glycine or proline
RXRRXRILIQYXRWKWHKX

SEQ ID NO: 518 - protein, 19 aa, synthetic construct
  REGION 1..19: note = cell penetrating peptide
  MOD_RES 2..5: note = Xaa = 6-aminohexanoic acid
  SITE 12: note = misc_feature - Xaa can be any naturally occurring amino acid
  MOD_RES 19: note = Xaa = glycine or proline
RXRRXRILIQYXRMKWHKX

SEQ ID NO: 519 - protein, 19 aa, synthetic construct
  REGION 1..19: note = cell penetrating peptide
  MOD_RES 2..5: note = Xaa = 6-aminohexanoic acid
  SITE 13: note = misc_feature - Xaa can be any naturally occurring amino acid
  SITE 17: note = misc_feature - Xaa can be any naturally occurring amino acid
  MOD_RES 19: note = Xaa = glycine or proline
RXRRXRILIQYRXRARXRX

SEQ ID NO: 520 - protein, 19 aa, synthetic construct
  REGION 1..19: note = cell penetrating peptide
  MOD_RES 2..5: note = Xaa = 6-aminohexanoic acid
  SITE 13: note = misc_feature - Xaa can be any naturally occurring amino acid
  SITE 15: note = misc_feature - Xaa can be any naturally occurring amino acid
  SITE 17: note = misc_feature - Xaa can be any naturally occurring amino acid
  MOD_RES 19: note = Xaa = glycine or proline
RXRRXRILIQYRXRXRXRX

SEQ ID NO: 521 - protein, 18 aa, synthetic construct
  REGION 1..18: note = cell penetrating peptide
  MOD_RES 2..5: note = Xaa = 6-aminohexanoic acid
  SITE 13: note = misc_feature - Xaa can be any naturally occurring amino acid
  SITE 16: note = misc_feature - Xaa can be any naturally occurring amino acid
  MOD_RES 18: note = Xaa = glycine or proline
RXRRXRILIQYRXRRXRX

SEQ ID NO: 522 - protein, 19 aa, synthetic construct
  REGION 1..19: note = cell penetrating peptide
  MOD_RES 2..5: note = Xaa = 6-aminohexanoic acid
  SITE 15: note = misc_feature - Xaa can be any naturally occurring amino acid
  MOD_RES 19: note = Xaa = glycine or proline
RXRRXRILIQYRARXRARX

SEQ ID NO: 523 - protein, 17 aa, synthetic construct
  REGION 1..17: note = cell penetrating peptide
  MOD_RES 2..5: note = Xaa = 6-aminohexanoic acid
  SITE 13: note = misc_feature - Xaa can be any naturally occurring amino acid
  MOD_RES 17: note = Xaa = glycine or proline
RXRRXRILIQYRXRARX

SEQ ID NO: 524 - protein, 18 aa, synthetic construct
  REGION 1..18: note = cell penetrating peptide
  MOD_RES 4..14: note = Xaa = 6-aminohexanoic acid
  MOD_RES 18: note = Xaa - glycine or proline
PRPXXXXXXXXXXXPRGX

SEQ ID NO: 525 - protein, 9 aa, synthetic construct
  REGION 1..9: note = cell penetrating peptide
  MOD_RES 9: note = Xaa = glycine or proline
RRRRRRRRX

SEQ ID NO: 526 - protein, 8 aa, synthetic construct
  REGION 1..8: note = cell penetrating peptide
  MOD_RES 8: note = Xaa = glycine or proline
RRMKWKKX

SEQ ID NO: 527 - protein, 8 aa, synthetic construct
  REGION 1..8: note = cell penetrating peptide
  MOD_RES 8: note = Xaa = glycine or proline
PKKKRKVX

SEQ ID NO: 528 - protein, 33 aa, synthetic construct
  REGION 1..33: note = cell penetrating peptide
  MOD_RES 33: note = Xaa = glycine or proline
CKDEPQRRSARLSAKPAPPKPEPKPKKAPAKKX

SEQ ID NO: 529 - protein, 10 aa, synthetic construct
  REGION 1..10: note = cell penetrating peptide
  MOD_RES 10: note = Xaa = glycine or proline
RKKRRQRRRX

SEQ ID NO: 530 - protein, 9 aa, synthetic construct
  REGION 1..9: note = cell penetrating peptide
  MOD_RES 9: note = Xaa = glycine or proline
RKKRRQRRX

SEQ ID NO: 531 - protein, 8 aa, synthetic construct
  REGION 1..8: note = cell penetrating peptide
  MOD_RES 8: note = Xaa = glycine or proline
RKKRRQRX

SEQ ID NO: 532 - protein, 9 aa, synthetic construct
  REGION 1..9: note = cell penetrating peptide
  MOD_RES 9: note = Xaa = glycine or proline
KKRRQRRRX

SEQ ID NO: 533 - protein, 9 aa, synthetic construct
  REGION 1..9: note = cell penetrating peptide
  MOD_RES 9: note = Xaa = glycine or proline
KKRRQRRRX

SEQ ID NO: 534 - protein, 10 aa, synthetic construct
  REGION 1..10: note = cell penetrating peptide
  MOD_RES 10: note = Xa = glycine or proline
AKKRRQRRRX

SEQ ID NO: 535 - protein, 10 aa, synthetic construct
  REGION 1..10: note = cell penetrating peptide
  MOD_RES 10: note = Xaa = glycine or proline
RAKRRQRRRX

SEQ ID NO: 536 - protein, 10 aa, synthetic construct
  REGION 1..10: note = cell penetrating peptide
  MOD_RES 10: note = Xaa = glycine or proline
RKARRQRRRX

SEQ ID NO: 537 - protein, 10 aa, synthetic construct
  REGION 1..10: note = cell penetrating peptide
  MOD_RES 10: note = Xaa = glycine or proline
RKKARQRRRX

SEQ ID NO: 538 - protein, 11 aa, synthetic construct
  REGION 1..11: note = cell penetrating peptide
  MOD_RES 11: note = Xaa = glycine or proline
CRWRWKCCKKX

SEQ ID NO: 539 - protein, 23 aa, synthetic construct
  REGION 1..23: note = Synthetic peptide
  MOD_RES 3: note = Xaa = D-arginine
  MOD_RES 7: note = Xaa = D-arginine
  MOD_RES 14: note = Xaa = D-arginine
  MOD_RES 18: note = Xaa = D-arginine
  MOD_RES 22: note = Xaa = D-arginine
YAXVRRXGPRGYAXVRRXGPRXB

SEQ ID NO: 540 - DNA, 25 bp, synthetic construct
  misc_feature 1..25: note = Synthetic nucleic acid
  source 1..25: mol_type = other DNA
cctcttacctcagttacaatttata